(19) United States
(12) Patent Application Publication
(10) Pub. No.: US 2020/0197536 A1
(43) Pub. Date: Jun. 25, 2020

(54) POROUS NANOPARTICLE-SUPPORTED LIPID BILAYER DELIVERY OF TRANSCRIPTIONAL GENE MODULATORS

(71) Applicants: Charles Jeffrey Brinker, Brinker, NM (US); Jacob Ongudi Agola, Albuquerque, NM (US); Ayse Muniz, Albuquerque, NM (US); Kimberly Butler, Albuquerque, NM (US)

(72) Inventors: Charles Jeffrey Brinker, Brinker, NM (US); Jacob Ongudi Agola, Albuquerque, NM (US); Ayse Muniz, Albuquerque, NM (US); Kimberly Butler, Albuquerque, NM (US)

(21) Appl. No.: 16/500,349

(22) PCT Filed: Apr. 3, 2018
(86) PCT No.: PCT/US2018/025830

§ 371 Date: Oct. 2, 2019

Related U.S. Application Data
(60) Provisional application No. 62/480,832, filed on Apr. 3, 2017.

(51) Int. Cl. A61K 48/00; A61K 9/51; C12N 15/85; C12Q 1/6886; C12N 9/22; C12N 15/11; A61P 35/00

(52) U.S. Cl. CPC A61K 48/0033; A61K 9/5115; A61K 9/5123; C12N 15/85; C12Q 1/6886; C12N 2800/10; C12N 15/111; A61P 35/00; C12Q 2600/118; C12N 2310/20; C12N 2800/80; C12N 9/22

(57) ABSTRACT

A porous protocell is provided comprising one or more non-integrating vectors comprising nucleic acid encoding a non-nucleolytic fusion protein comprising a protein sequence that specifically binds a sequence in a double strand nucleic acid molecule linked to a protein sequence that is a transcriptional mediator, and optionally guide RNA or a vector for expression of guide RNA, and methods of using the protocells.

### CROSS-REFERENCE TO RELATED APPLICATIONS

The present application claims the benefit of the filing date of U.S. application Ser. No. 62/480,832, filed on Apr. 3, 2017, the disclosure of which is incorporated by reference herein.

### BACKGROUND

Targeted delivery of drugs encapsulated within nanocarriers can potentially ameliorate a number of problems exhibited by conventional ‘free’ drugs, including poor solubility, limited stability, rapid clearing, and, in particular, lack of selectivity, which results in non-specific toxicity to normal cells and prevents the dose escalation necessary to eradicate diseased cells. Passive targeting schemes, which rely on the enhanced permeability of the tumor vasculature and decreased draining efficacy of tumor lymphatics to direct accumulation of nanocarriers at tumor sites (the so-called enhanced permeability and retention, or EPR effect), overcome many of these problems, but the lack of cell-specific interactions needed to induce nanocarrier internalization decreases therapeutic efficacy and can result in drug expulsion and induction of multiple drug resistance.

One of the challenges in nanomedicine is to engineer nanostructures and materials that can efficiently encapsulate cargo, for example, drugs, at high concentration, cross the cell membrane, and controllably release the drugs at the target site over a prescribed period of time. Recently, inorganic nanoparticles have emerged as a new generation of drug or therapy delivery vehicles in nanomedicine. More recently, gating methods that employ coumarin, azobenzene, rotaxane, polymers, or nanoparticles have been developed to seal a cargo within a particle and allow a triggered release according to an optical or electrochemical stimulus.

While liposomes have been widely used in drug delivery due to their low immunogenicity and low toxicity, they still need to be improved in several aspects. First, the loading of cargo can only be achieved under the condition in which liposomes are prepared. Therefore, the concentration and category of cargo may be limited. Second, the stability of liposomes is relatively low. The lipid bilayer of the liposomes often tends to age and fuse, which changes their size and size distribution. Third, the release of cargo in liposomes is instantaneous upon rupture of the liposome which makes it difficult to control the release.

A porous nanoparticle-supported lipid bilayer (protocell), formed via fusion of liposomes to nanoporous silica particles, is a novel type of nanocarrier that addresses multiple challenges associated with targeted delivery of cancer therapeutics and diagnostics. Like liposomes, protocells are biocompatible, biodegradable, and non-immunogenic, but their nanoporous silica core confers a drastically enhanced cargo capacity and prolonged bilayer stability when compared to similarly-sized liposomal delivery agents. The porosity and surface chemistry of the core can, furthermore, be modulated to promote encapsulation of a wide variety of therapeutic agents, such as drugs, nucleic acids, and protein toxins. The rate of cargo release can be controlled by pore size, chemical composition and the overall degree of silica condensation of the core, making protocells useful in applications requiring either burst or controlled release profiles. Finally, the protocells supported lipid bilayer (SLB) can be modified with variously with ligands to promote selective delivery and with PEG to extend circulation times.

The need to improve the activity of chemotherapeutic agents and to enhance cancer therapy is ongoing. The use of protocells in conjunction with alternative approaches to targeting, binding, enhancing invasion of cancer and depositing chemotherapeutic agents in proximity to their site of activity are important facets of cancer therapy. The present disclosure is undertaken to advance the art of cancer therapy and to improve the delivery of agents which can influence therapeutic outcome, whether by enhancing the administration of cancer therapeutic agents or in diagnostics, to facilitate approaches to diagnosing cancer and monitoring cancer therapy.

### SUMMARY

The protocell platform, which consists of a mesoporous silica nanoparticle (MSNP) core wrapped in a supported lipid bilayer (SLB), can be utilized to deliver nucleic acid vectors, such as plasmids, which provide short-term modulation of target genes. The ability to use the protocell with its facile MSNP core, allows the use of fluorescent MSNPs to allow separate tracking of particle delivery and gene modulation effect. Additionally, the use of the MSNP core with its high pore volume, allows the inclusion of drugs to be tested for additive or synergistic effect with the gene modulation. The use of the SLB allows creation of both specifically targeted (to a particular cell or organ of interest) or universal, non-targeted delivery by modulating the formulation of the SLB. Additionally, the use of the SLB protects the nucleic acid, e.g., a non-replicating or non-integrative plasmid, cargo from degradation within a biological system and the combination of the MSNP plus the SLB creates an extra-stable carrier for plasmid delivery. Due to the use of vectors, such as plasmids that do not integrate into the host genome, this modulation, either activation or repression, has a limited duration of effect within a host cell or organism. Additionally, due to the modular nature of the protocell, any transcriptional activator or repressor plasmid system targeting any gene of interest may be loaded. Finally, the ability to modify the size of the MSNP and the lipid composition on the surface of the protocell allows both active and passive targeting to specific organs or cells within the body.

By providing short-term modulation, either in vitro or in vivo, genes and their target proteins can be up-regulated or down-regulated for short periods of time (on the order of a few days to weeks). For example, short-term modulation in vitro allows for in vitro analysis of short-term perturbations in gene or protein levels and effects of subsequent recovery. This allows assessment of the effects of major genes; such as drug resistance, drug metabolism or apoptotic genes, in any cell line without the need to permanently alter the cell line through viral infection. The ability to use any cell line expands the number cell lines that can be utilized for disease research. The ability to use the protocell for delivery allows a fluorescent MSNP core for tracking of particle delivery and potentially sorting of cell based off of particle association. Additionally, the use of the MSNP core allows the pore structure of the core to be dual loaded with additional therapeutics for more complex testing.

In an in vivo model system, such as a mouse, the use of the protocell delivery of transcriptional modulators can be utilized to model a change in disease part way through an experiment. For example, a xenograft tumor grown in a mouse could then be altered for a few weeks to express an increased or decreased level of a drug resistance gene. The protocell allows the potential of targeted delivery of the gene modulators to the xenograft tumor or non-specific delivery to the mouse model system. Similar to the in vitro system, the MSNP core can be fluorescently or radio labeled for real time tracking of the particle biodistribution as well as provide drug cargos.

In a therapeutic context, the protocell delivery of transcriptional modulators can be either targeted by ligands on the SLB or non-specific delivery to the entire organism. Since the modulation is plasmid based and therefore does not involve alteration of the host genome through integration or editing, the effects are transient but should be repeatable with additional doses of the protocell. The advantage of utilizing a system that does not integrate or edit is that it removes the possibility of creating a new disease through error. The transient nature also means that a therapeutic gene alteration can be tested on a patient without permanents changes, to select a dose and amount that is personalized for the individual, or treat a transient condition that would not require permanent alteration of the gene expression.

In one embodiment, the MSNP with SLB comprises one or more nucleic acid, e.g., DNA, vectors. In one embodiment, the SLB comprises DOTAP, e.g., from about 40% to about 100% by weight DOTAP, including from about 50% to about 60% DOTAP by weight. In one embodiment, the SLB comprises DOPE, e.g., from about 1% to about 10% by weight DOPE, including from about 2% to about 8% DOPE by weight. In one embodiment, the SLB comprises cholesterol, e.g., from about 20% to about 40% by weight, including from about 25% to about 35% cholesterol by weight. In one embodiment, the SLB comprises a PEGylated lipid such as PEGylated DOPE or PEGylated DSPE, e.g., from about 5% to about 40% or about 7% to about 20% by weight PEGylated lipid. In one embodiment, a vector encodes a reporter protein. In one embodiment, a vector encodes a nucleolytically inactive protein that allows for DNA binding, e.g., sequence specific binding, linked to a domain that allows for transcriptional activation or repression. In one embodiment, a porous protocell comprises a nanoporous silica or metal oxide core with a supported lipid bilayer and at least one of one or more non-integrating vectors comprising nucleic acid encoding a non-nucleolytic fusion protein comprising a protein sequence that specifically binds a sequence in a double strand nucleic acid molecule linked to a protein sequence that is a transcriptional mediator, guide RNA or a vector for expression of guide RNA, and an activator or repressor helper protein for the transcprtional mediator.

In one embodiment, nonspecific protocells are employed, e.g., protocells made with a SLB that includes cationic lipids useful to deliver plasmids to any cell they encounter. Examples of these formulations include but are not limited to 1,2-dioleoyl-3-trimethylammonium-propane (DOTAP) based formulations which can range from 30-100% DOTAP and can utilize other lipids such as 1,2-dioleoyl-sn-glycero-3-phosphoethanolamine (DOPE) to alter membrane fluidity and reduce toxicity and PEGylated lipids to increase stability in biological systems.

In one embodiment, specific protocells are employed, e.g., protocells having a SLB that is functionalized for the addition of antibodies, affibodies, single chain antibodies or peptides to allow targeting to specific proteins on a cell surface. This allows targeting to specific cells (such as diseased cells) or organs.

### BRIEF DESCRIPTION OF THE FIGURES

FIG. 1. Flow cytometry quantification of the delivery and subsequent expression of GFP plasmid by 3 non-specific formulations of protocells.

FIG. 2. Fluorescence Microscropy of p53 expression in control cells (left) and after treatment with SAM loaded protocells (right). The nuclei of the cells are stained with Hoecht (blue) and the protocells (red) are visualized associated with the cells. The p53 protein was stained with an antibody to p53 (green) and the treatment with the SAM shows a significant increase in p53 expression.

FIG. 3. Quantification of the cells expressing p53 significantly above the background level by flow cytometry. Protein p53 was stained by antibody and the expression was quantified by flow cytometry.

FIGS. 4A-B. Live/Dead Flow cytometry of cells treated with Protocells alone, Protocells loaded with the SAM, the recommended transfection agent for SAM (control SAM transfection) or left untreated.

FIG. 5. Flow cytometry quantification of the delivery and subsequent expression of GFP plasmid by 6 targeted formulations of protocells.

FIG. 6. Solutions provided by protocells.

FIG. 7. Schematic of preparation and use of protocells having plasmid DNA in the core.

FIGS. 8A-8C. A) Schematic of SAM components. B) Schematic of CRISPR reportr plasmid system. C) Uses of CRISPR.

FIG. 9. Targeted delivery of protocells.

FIG. 10. CRISPR SAM system. Either plasmids that encode for the system or the SAM complex components can be delivered to the target cell to drive increased expression of any gene of interest.

FIG. 11. Schematic of the protocell. Protocells are formed by soaking spherical, porous silica nanoparticles in the cargo(s) of interest, e.g. individual CRISPR components or plasmids. PEGylated liposomes are then fused to cargo-loaded cores, resulting in a supported lipid bilayer that enhances colloidal stability and helps to retain encapsulated cargo. The SLB can be further modified with targeting ligands that enable molecule-specific binding using a number of cross-linking strategies, e.g biotin-streptavidin, CLICK, NHS ester-maleimide, etc.

FIG. 12. Microscopic evaluation of increased p53 expression after delivery of p53 CRISPR SAM construct by protocells. The background image (left) shows the background level of p53 protein. The image on the right shows the presence of protocells (red) and increased p53 protein expression 72 hours after treatment with CRISPR SAM construct loaded protocells.

FIG. 13. Schematic showing the effect of endurance training or high altitude on red blood cell production. Endurance training or high altitude exposure lead to increases in HIF-1 protein levels which result in synthesis of EPO in the kidney. EPO is secreted from the kidneys and travels via the vasuclature to the bone marrow. EPO secreted from the kidneys results in increased production of red blood cells. Endurance training and high altitude training also increase HIF-1 in the bone marrow and cause erythrocyte precursors to increase transferrin receptor expression necessary for hemoglobin and erythrocyte production.

FIG. 14. Schematic of the experimental plan for identification of sucessful HIF-1, EPO and MCT1 targeted CRISPR SAM systems. Individual SAM systems will be created for HIF-1, EPO and MCT1. These systems are transfected into target cells. Protein and mRNA are then extracted and assessed for expression of both the direct target and downstream targets using quantified western blot and qRT-PCR.

FIGS. 15A-B. Schematics representing the additional genes activated by high altitude and the experimental plan for identification of sucessful ANGPLT4 and apeline targeted CRISPR SAM systems. A) Exposure to high altitude results in decreased O₂ in the body and results in increased expression of apelin and ANGPTL4 in kidney, muscle and liver. Apelin and ANGPTL4 are released into the blood stream and provide an adaptive and protective response to high altitude. B) Individual SAM systems will be created for apelin and ANGPTL4, and these systems are transfected into target cells. Protein and mRNA are then be extracted and assessed for expression of both the direct target and downstream targets using quantified western blot and qRT-PCR.

FIGS. 16A-B. Schematics representing the genes activated by extreme cold and the experimental plan for identification of sucessful FNDC5 and FGF21 targeted CRISPR SAM systems. A) Exposure to extreme cold results in decreased body temperature and results in increased expression of FNDC5 in muscle and FGF21 in liver, FGF21 and FNDC5 are released into the blood stream and cause an increase in internal heat production in adipose tissue. B) Individual SAM systems are created for FGF21 and FNDC5 and these systems are transfected into target cells. Protein and mRNA are then extracted and assessed for expression of both the direct target and downstream targets using quantified western blot and qRT-PCR.

FIGS. 17A-D. Transfection of pMAX-GFP plasmid into cells utilizing both targeted and non-targeted protocells. A) Percentage of cells positive for GFP after targeted delivery of GFP plasmid to cells expressing Her2 on the cell surface. Non-targeted liposomes or protocells showed no transfection, while targeted delivery showed specific transfection in up to 67% of cells. B) Non-specific delivery using a cationic lipid (DOTAP) formulation in two different cell lines showing comparable transfection to lipofectamine, but very low transfection by the lipids alone. C) DOTAP protocells loaded with GFP plasmid were injected into the vasculature of the chicken chorioallantioc membrane and demonstrated transfection after 24 hours. D) Enlargement of the white box in C showing a region of transfection.

FIGS. 18A-C. A) The embryo and CAM can be maintained ex ovo (out of their shell), allowing for easy access to the vascular network of the CAM. B) Detailed image of the vascular network of the CAM showing the complexity of the vascular system. C)

Multiphoton imaging of the CAM vascular network which provides 3D imaging of the vascular system.

FIGS. 19A-B. Schematic of non-specific protocell creation and experimental design for development of non-specific protocells for CRISPR SAM delivery. A) Mesoporous silica nanoparticles are loaded with plasmid and then coated with lipids.

The lipid formulation can be varied based on the experimental results. B) Cells will be exposed to protocells loaded with pMAX-GFP plasmid and samples will be collected at 24, 48 and 72 hours. Samples will be assessed by flow cytometry for GFP production and for alterations in cell viability.

FIGS. 20A-B. Schematic of targeted protocell creation and experimental design for development of cell type specific protocells for CRISPR SAM delivery. A) Mesoporous silica nanoparticles are loaded with plasmid and then coated with lipids. Once coated, targeting moieties such as antibodies, affibodies, small molecules and peptides can be conjugated to the surface. The lipid formulation and the choice of targeting moiety can be varied based on the experimental results. B) Cells are exposed to protocells loaded with pMAX-GFP plasmid and samples will be collected at 4, 24, 48 and 72 hours. Samples are assessed by microscopy for protocell binding, flow cytometry for GFP production and for alterations in cell viability.

FIGS. 21A-B. Use of the CAM model to evaluate protocell flow characteristics, targeting and cargo delivery to specific cells in a vascularized system. A) Red fluorescent monosize, stable protocells and polydisperse, unstable protocells were injected into the CAM vasculature and assessed for clearance and non-specific interaction with endothelial cells. Stable protocells demonstrated long-term circulation and low non-specific interactions, while the unstable protocells demonstrated rapid clearance and high non-specific interactions. B) Binding of protocells (red) to targeted cells (blue) within the CAM vascular system (purple) is rapid and remains strong at 4 hours post injection. Delivery of a membrane impermenat dye (Green) is visible by 16 hours post injection.

FIG. 22. Schematic of the overall experimental plan for testing functional alterations in target cells and downstream cells of the CRISPR SAM systems delivered by protocells. Transfected cells will secrete proteins in response to the CRIPSR SAM transfection into the media to create cell conditioned media. Both the target cells and downstream cells are stressed by the appropriate stressor and assessed for cell viability and cell metabolism alterations.

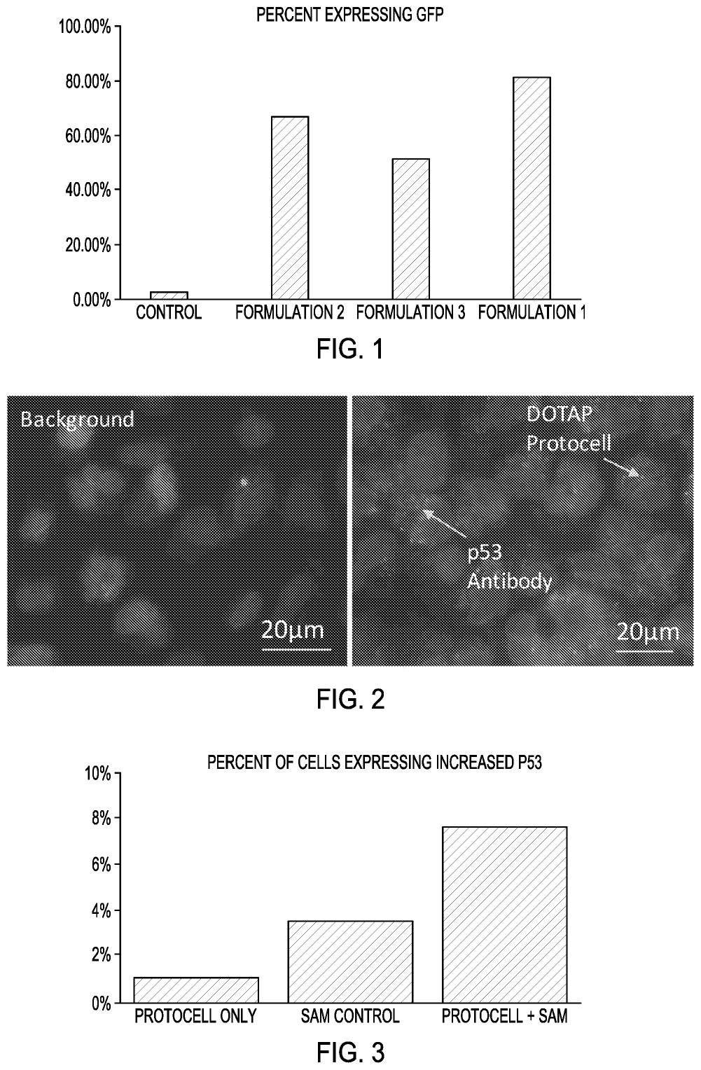

### DETAILED DESCRIPTION

The following terms shall be used throughout the specification to describe the present invention. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially including a domesticated animal or a human, to whom treatment, including prophylactic treatment (prophylaxis), with the compounds or compositions according to the present disclosure is provided. For treatment of those infections, conditions or disease states which are specific for a specific animal such as a human patient, the term patient refers to that specific animal. In most instances, the patient or subject of the present disclosure is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diasteromers), individual optical isomers (enantiomers) or racemic mixtures, pharmaceutically acceptable salts and prodrug forms. The term compound herein refers to stable compounds. Within its use in context, the term compound may refer to a single compound or a mixture of compounds as otherwise described herein.

The term “bioactive agent” refers to any biologically active compound or drug which may be formulated for use in an embodiment of the present disclosure. Exemplary bioactive agents include the compounds according to the present disclosure which are used to treat cancer or a disease state or condition which occurs secondary to cancer and may include antiviral agents, especially anti-HIV, anti-HBV and/or anti-HCV agents (especially where hepatocellular cancer is to be treated) as well as other compounds or agents which are otherwise described herein.

The terms “treat”, “treating”, and “treatment”, are used synonymously to refer to any action providing a benefit to a patient at risk for or afflicted with a disease, including improvement in the condition through lessening, inhibition, suppression or elimination of at least one symptom, delay in progression of the disease, prevention, delay in or inhibition of the likelihood of the onset of the disease, etc. In the case of viral infections, these terms also apply to viral infections and include, in certain particular embodiments the eradication or elimination (as provided by limits of diagnostics) of the virus which is the causative agent of the infection.

Treatment, as used herein, encompasses both prophylactic and therapeutic treatment, principally of cancer, but also of other disease states, including viral infections, especially including HBV and/or HCV. Compounds according to the present disclosure can, for example, be administered prophylactically to a mammal in advance of the occurrence of disease to reduce the likelihood of that disease. Prophylactic administration is effective to reduce or decrease the likelihood of the subsequent occurrence of disease in the mammal, or decrease the severity of disease (inhibition) that subsequently occurs, especially including metastasis of cancer. Alternatively, compounds according to the present disclosure can, for example, be administered therapeutically to a mammal that is already afflicted by disease. In one embodiment of therapeutic administration, administration of the present compounds is effective to eliminate the disease and produce a remission or substantially eliminate the likelihood of metastasis of a cancer. Administration of the compounds according to the present disclosure is effective to decrease the severity of the disease or lengthen the lifespan of the mammal so afflicted, as in the case of cancer, or inhibit or even eliminate the causative agent of the disease, as in the case of hepatitis B virus (HBV) and/or hepatitis C virus infections (HCV) infections.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

The term “inhibit” as used herein refers to the partial or complete elimination of a potential effect, while inhibitors are compounds/compositions that have the ability to inhibit.

The term “prevention” when used in context shall mean “reducing the likelihood” or preventing a disease, condition or disease state from occurring as a consequence of administration or concurrent administration of one or more compounds or compositions according to the present disclosure, alone or in combination with another agent. It is noted that prophylaxis will rarely be 100% effective; consequently the terms prevention and reducing the likelihood are used to denote the fact that within a given population of patients or subjects, administration with compounds according to the present disclosure will reduce the likelihood or inhibit a particular condition or disease state (in particular, the worsening of a disease state such as the growth or metastasis of cancer) or other accepted indicators of disease progression from occurring.

The term “protocell” is used to describe a porous nanoparticle which is made of a material comprising silica, polystyrene, alumina, Mania, zirconia, or generally metal oxides, organometallates, organosilicates or mixtures thereof. A porous spherical silica nanoparticle is used for certain embodiments of protocells and is surrounded by a supported lipid or polymer bilayer or multilayer. Various embodiments according to the present disclosure provide nanostructures and methods for constructing and using the nanostructures and providing protocells according to the present disclosure. Many of the protocells in their most elemental form are known in the art. Porous silica particles of varying sizes ranging in size (diameter) from less than 5 nm to 200 nm or 500 nm or more are readily available in the art or can be readily prepared using methods known in the art (see the examples section) or alternatively, can be purchased from Melorium Technologies, Rochester, N.Y. SkySpring Nano materials, Inc., Houston, Tex., USA or from Discovery Scientific, Inc., Vancouver, British Columbia. Multimodal silica nanoparticles may be readily prepared using the procedure of Carroll, et al., Langmuir, 25, 13540-13544 (2009). Protocells can be readily obtained using methodologies known in the art. The examples section of the present application provides certain methodology for obtaining protocells which are useful in the present disclosure. Protocells according to the present disclosure may be readily prepared, including protocells comprising lipids which are fused to the surface of the silica nanoparticle. See, for example, Liu, et al., Chem. Comm., 5100-5102 (2009), Liu, et al., J. Amer. Chem. Soc., 131, 1354-1355 (2009), Liu, et al., J. Amer. Chem, Soc., 131, 7567-7569 (2009) Lu, et al., Nature, 398, 223-226 (1999), Exemplary protocells for use in the present disclosure are prepared according to the procedures which are presented in Ashley, et al., Nature Materials, 2011, May;10(5):389-97, Lu, et al., Nature, 398, 223-226 (1999), Caroll, et al., Langmuir, 25, 13540-13544 (2009), and as otherwise presented in the experimental section which follows.

In an embodiment of the present disclosure, the nanostructures include a core-shell structure which comprises a porous particle core surrounded by a shell of lipid for example a bilayer, but possibly a monolayer or multilayer (see Liu, et al., JACS, 2009, Id). The porous particle core can include, for example, a porous nanoparticle made of an inorganic and/or organic material as set forth above surrounded by a lipid bilayer. In the present disclosure, these lipid bilayer surrounded nanostructures are referred to as “protocells” or “functional protocells,” since they have a supported lipid bilayer membrane structure. In embodiments according to the present disclosure, the porous particle core of the protocells can be loaded with various desired species (“cargo”), including small molecules (e.g. anticancer agents as otherwise described herein), large molecules (e.g. including macromolecules such as RNA, including small interfering RNA or siRNA or small hairpin RNA or shRNA or a polypeptide which may include a polypeptide toxin such as a ricin toxin A-chain or other toxic polypeptide such as diphtheria toxin A-chain DTx, among others) or a reporter polypeptide (e.g. fluorescent green protein, among others) or semiconductor quantum dots, or metallic nanparticles, or metal oxide nanoparticles or combinations thereof. In certain aspects of the disclosure, the protocells are loaded with super-coiled plasmid DNA, which can be used to deliver a therapeutic and/or diagnostic peptide(s) or a small hairpin RNA/shRNA or small interfering RNA/siRNA which can be used to inhibit expression of proteins (such as, for example growth factor receptors or other receptors which are responsible for or assist in the growth of a cell especially a cancer cell, including epithelial growth factor/EGFR, vascular endothelial growth factor receptor/VEGFR-2 or platelet derived growth factor receptor/PDGFR-α, among numerous others, and induce growth arrest and apoptosis of cancer cells).

In certain embodiments, the cargo components can include, but are not limited to, chemical small molecules (especially anticancer agents and antiviral agents, including anti-HIV, anti-HBV and/or anti-HCV agents, nucleic acids (DNA and RNA, including siRNA and shRNA and plasmids which, after delivery to a cell, express one or more polypeptides or RNA molecules), such as for a particular purpose, such as a therapeutic application or a diagnostic application as otherwise disclosed herein.

In embodiments, the lipid bilayer of the protocells can provide biocompatibility and can be modified to possess targeting species including, for example, targeting peptides including antibodies, aptamers, and PEG (polyethylene glycol) to allow, for example, further stability of the protocells and/or a targeted delivery into a bioactive cell.

The protocells particle size distribution, according to the present disclosure, depending on the application, may be monodisperse or polydisperse. The silica cores can be rather monodisperse (i.e., a uniform sized population varying no more than about 5% in diameter e.g., ±10-nm for a 200 nm diameter protocell especially if they are prepared using solution techniques) or rather polydisperse (i.e., a polydisperse population can vary widely from a mean or medium diameter, e.g., up to ±200-nm or more if prepared by aerosol. Polydisperse populations can be sized into monodisperse populations. All of these are suitable for protocell formation. In the present disclosure, exemplary protocells are no more than about 500 nm in diameter, or no more than about 200 nm in diameter in order to afford delivery to a patient or subject and produce an intended therapeutic effect.

Protocells according to the present disclosure generally range in size from greater than about 8-10 nm to about 5 μm in diameter, for example, about 20-nm-3 μm in diameter, about 10 nm to about 500 nm, or about 20-200-nm (including about 150 nm, which may be a mean or median diameter). As discussed above, the protocell population may be considered monodisperse or polydisperse based upon the mean or median diameter of the population of protocells. Size is very important to therapeutic and diagnostic aspects of the present disclosure as particles smaller than about 8-nm diameter are excreted through kidneys, and those particles larger than about 200 nm are trapped by the liver and spleen . Thus, an embodiment of the present disclosure focuses in smaller sized protocells for drug delivery and diagnostics in the patient or subject.

Protocells according the present disclosure are characterized by containing mesopores, such as pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. Exemplary pore sizes of mesopores range from about 2-30 nm; they can be monosized or bimodal or graded they can be ordered or disordered (essentially randomly disposed or worm-like).

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm e.g. if a templating moiety in the aerosol process is not used. They could also be enlarged to macropores, i.e., 50-nm in diameter.

Pore surface chemistry of the nanoparticle material can be very diverse—all organosilanes yielding cationic, anionic, hydrophilic, hydrophobic, reactive groups—pore surface chemistry, especially charge and hydrohobicity, affect loading capacity. Attractive electrostatic interactions or hydrophobic interactions control/enhance loading capacity and control release rates. Higher surface areas can lead to higher loadings of drugs/cargos through these attractive interactions. See below.

The surface area of nanoparticles, as measured by the N2 BET method, ranges from about 100 m2/g to >about 1200 m2/g. In general, the larger the pore size, the smaller the surface area. The surface area theoretically could be reduced to essentially zero, if one does not remove the templating agent or if the pores are sub-0.5-nm and therefore not measurable by N2 sorption at 77K due to kinetic effects. However, in this case, they could be measured by CO2 or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the protocells according to the present disclosure are loaded with cargo to a capacity up to about 50 weight %: defined as (cargo weight/weight of loaded protocell)×100. The optimal loading of cargo is often about 0.01 to 10% but this depends on the drug or drug combination which is incorporated as cargo into the protocell. This is generally expressed in μM per 10¹⁰ particles where we have values ranging from 2000-100 μM per 10¹⁰ particles. Exemplary protocells according to the present disclosure exhibit release of cargo at pH about 5.5, which is that of the endosome, but are stable at physicological pH of 7 or higher (7.4).

The surface area of the internal space for loading is the pore volume whose optimal value ranges from about 1.1 to 0.5 cubic centimeters per gram (cc/g). Note that in the protocells according to one embodiment of the present disclosure, the surface area is mainly internal as opposed to the external geometric surface area of the nanoparticle.

The lipid bilayer supported on the porous particle according to one embodiment of the present disclosure has a lower melting transition temperature, i.e. is more fluid than a lipid bilayer supported on a non-porous support or the lipid bilayer in a liposome. This is sometimes important in achieving high affinity binding of targeting ligands at low peptide densities, as it is the bilayer fluidity that allows lateral diffusion and recruitment of peptides by target cell surface receptors. One embodiment provides for peptides to cluster, which facilitates binding to a complementary target.

In the present disclosure, the lipid bilayer may vary significantly in composition. Ordinarily, any lipid or polymer which is may be used in liposomes may also be used in protocells. Exemplary lipids are as otherwise described herein. Exemplary lipid bilayers for use in protocells according to the present disclosure comprise a mixtures of lipids (as otherwise described herein) at a weight ratio of 5% DOPE, 5% PEG, 30% cholesterol, 60% DOPC or DPPC (by weight).

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the protocell. Generally, after fusion of the supported lipid bilayer, the zeta-potential is reduced to between about −10 mV and +5 mV, which is important for maximizing circulation time in the blood and avoiding non-specific interactions.

Depending on how the surfactant template is removed, e.g. calcination at high temperature (500° C.) versus extraction in acidic ethanol, and on the amount of AEPTMS incorporated in the silica framework, the silica dissolution rates can be varied widely. This in turn controls the release rate of the internal cargo. This occurs because molecules that are strongly attracted to the internal surface area of the pores diffuse slowly out of the particle cores, so dissolution of the particle cores controls in part the release rate.

Further characteristics of protocells according to an embodiment of the present disclosure are that they are stable at pH 7, i.e. they don't leak their cargo, but at pH 5.5, which is that of the endosome lipid or polymer coating becomes destabilized initiating cargo release. This pH-triggered release is important for maintaining stability of the protocell up until the point that it is internalized in the cell by endocytosis, whereupon several pH triggered events cause release into the endosome and consequently, the cytosol of the cell. Quantitative experimental evidence has shown that targeted protocells illicit only a weak immune response, because they do not support T-Cell help required for higher affinity IgG, a favorable result.

Protocells according to the present disclosure exhibit at least one or more a number of characteristics (depending upon the embodiment) which distinguish them from prior art protocells:
1) In contrast to the prior art, an embodiment of the present disclosure specifies nanoparticles whose average size (diameter) is less than about 200-nm—this size is engineered to enable efficient cellular uptake by receptor mediated endocytosis;
2) An embodiment of the present disclosure can specify both monodisperse and/or polydisperse sizes to enable control of biodistribution.
3) An embodiment of the present disclosure is directed to targeted nanoparticles that induce receptor mediated endocytosis.
4) An embodiment of the present disclosure induces dispersion of cargo into cytoplasm through the inclusion of fusogenic or endosomolytic peptides.
5) An embodiment of the present disclosure provides particles with pH triggered release of cargo.
6) An embodiment of the present disclosure exhibits controlled time dependent release of cargo (via extent of thermally induced crosslinking of silica nanoparticle matrix).
7) An embodiment of the present disclosure can exhibit time dependent pH triggered release.
8) An embodiment of the present disclosure can contain and provide cellular delivery of complex multiple cargoes.
9) An embodiment of the present disclosure shows the killing of target cancer cells.
10) An embodiment of the present disclosure shows diagnosis of target cancer cells.
11) An embodiment of the present disclosure shows selective entry of target cells.
12) An embodiment of the present disclosure shows selective exclusion from off-target cells (selectivity).
13) An embodiment of the present disclosure shows enhanced enhanced fluidity of the supported lipid bilayer.
14) An embodiment of the present disclosure exhibits sub-nanomolar and controlled binding affinity to target cells.
15) An embodiment of the present disclosure exhibits sub-nanomolar binding affinity with targeting ligand densities below concentrations found in the prior art.
16) An embodiment of the present disclosure can further distinguish the prior art with with finer levels of detail unavailable in the prior art.

The term “lipid” is used to describe the components which are used to form lipid bilayers on the surface of the nanoparticles which are used in the present disclosure. Various embodiments provide nanostructures which are constructed from nanoparticles which support a lipid bilayer(s). In embodiments according to the present disclosure, the nanostructures include, for example, a core-shell structure including a porous particle core surrounded by a shell of lipid bilayer(s). The nanostructure, e.g., a porous silica nanostructure as described above, supports the lipid bilayer membrane structure. In embodiments according to the disclosure, the lipid bilayer of the protocells can provide biocompatibility and can be modified to possess targeting species including, for example, targeting peptides, fusogenic peptides, antibodies, aptamers, and PEG (polyethylene glycol) to allow, for example, further stability of the protocells and/or a targeted delivery into a bioactive cell, in particular a cancer cell. PEG, when included in lipid bilayers, can vary widely in molecular weight (although PEG ranging from about 10 to about 100 units of ethylene glycol, about 15 to about 50 units, about 15 to about 20 units, about 15 to about 25 units, about 16 to about 18 units, etc, may be used and the PEG component which is generally conjugated to phospholipid through an amine group comprises about 1% to about 20%, about 5% to about 15%, or about 10° k by weight of the lipids which are included in the lipid bilayer.

Numerous lipids which are used in liposome delivery systems may be used to form the lipid bilayer on nanoparticles to provide protocells according to the present disclosure. Virtually any lipid which is used to form a liposome may be used in the lipid bilayer which surrounds the nanoparticles to form protocells according to an embodiment of the present disclosure. Exemplary lipids for use in the present disclosure include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-racglycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}sn-glycero-3-phosphocholine (16:0-12:0 NBD PC), cholesterol and mixtures/combinations thereof. Cholesterol, not technically a lipid, but presented as a lipid for purposes of an embodiment of the present disclosure given the fact that cholesterol may be an important component of the lipid bilayer of protocells according to an embodiment of the disclosure. Often cholesterol is incorporated into lipid bilayers of protocells in order to enhance structural integrity of the bilayer. These lipids are all readily available commercially from Avanti Polar Lipids, Inc. (Alabaster, Ala., USA). DOPE and DPPE are particularly useful for conjugating (through an appropriate crosslinker) peptides, polypeptides, including antibodies, RNA and DNA through the amine group on the lipid.

The term “reporter” is used to describe an imaging agent or moiety which is incorporated into the phospholipid bilayer or cargo of protocells according to an embodiment of the present disclosure and provides a signal which can be measured. The moiety may provide a fluorescent signal or may be a radioisotope which allows radiation detection, among others. Exemplary fluorescent labels for use in protocells (e.g., via conjugation or adsorption to the lipid bilayer or silica core, although these labels may also be incorporated into cargo elements such as DNA, RNA, polypeptides and small molecules which are delivered to cells by the protocells, include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester(532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moities which enhance the fluorescent signal or slow the fluorescent fading may also be incorporated and include SlowFade® Gold antifade reagent (with and without DAPI) and Image-iT® FX signal enhancer. All of these are well known in the art. Additional reporters include polypeptide reporters which may be expressed by plasmids (such as histone-packaged supercoiled DNA plasmids) and include polypeptide reporters such as fluorescent green protein and fluorescent red protein. Reporters pursuant to the present disclosure are utilized principally in diagnostic applications including diagnosing the existence or progression of cancer (cancer tissue) in a patient and or the progress of therapy in a patient or subject.

The term “histone-packaged supercoiled plasmid DNA” is used to describe a component of protocells according to the present disclosure which utilize for example plasmid DNA which has been “supercoiled” (i.e., folded in on itself using a supersaturated salt solution or other ionic solution which causes the plasmid to fold in on itself and “supercoil” in order to become more dense for efficient packaging into the protocells). The plasmid may be virtually any plasmid which expresses any number of polypeptides or encode RNA, including small hairpin RNA/shRNA or small interfering RNA/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into protocells (either adsorbed into the pores or confined directly within the nanoporous silica core itself). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (i.e. complexing DNA with cationic lipid mixtures). In addition, DNA has also been packaged with cationic proteins (including proteins other than histones), as well as gold nanoparticles (e.g. NanoFlares—an engineered DNA and metal complex in which the core of the nanoparticle is gold).

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins may be used. In certain aspects of the disclosure, a combination of human histone proteins H1, H2A, H2B, H3 and H4 in a ratio of 1:2:2:2:2, although other histone proteins may be used in other, similar ratios, as is known in the art or may be readily practiced pursuant to the teachings of the present disclosure. The DNA may also be double stranded linear DNA, instead of plasmid DNA, which also may be optionally supercoiled and/or packaged with histones or other packaging components.

Other histone proteins which may be used in this aspect of the disclosure include, for example, H1F, H1F0, H1FNT, H1FOO, H1FX H1H1 HIST1H1A, HIST1H1B, HIST1H1C, HIST1H1D, HIST1H1E, HIST1H1T, H2AF, H2AFB1, H2AFB2, H2AFB3, H2AFJ, H2AFV, H2AFX, H2AFY, H2AFY2, H2AFZ, H2A1, HIST1H2AA, HIST1H2AB, HIST1H2AC, HIST1H2AD, HIST1H2AE, HIST1H2AG, HIST1H2Al, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF , HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST1H3C, HIST1H3D, HIST1H3E, HIST1H3F, HIST1H3G, HIST1H3H, HIST1H3I, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H4I, HIST1H4J, HIST1H4K, HIST1 H4L, H44 and HIST4H4.

The term “nuclear localization sequence” refers to a peptide sequence incorporated or otherwise crosslinked into histone proteins which comprise the histone-packaged supercoiled plasmid DNA. In certain embodiments, protocells according to the present disclosure may further comprise a plasmid (often a histone-packaged supercoiled plasmid DNA) which is modified (crosslinked) with a nuclear localization sequence (note that the histone proteins may be crosslinked with the nuclear localization sequence or the plasmid itself can be modified to express a nuclear localization sequence) which enhances the ability of the histone-packaged plasmid to penetrate the nucleus of a cell and deposit its contents there (to facilitate expression and ultimately cell death. These peptide sequences assist in carrying the histone-packaged plasmid DNA and the associated histones into the nucleus of a targeted cell whereupon the plasmid will express peptides and/or nucleotides as desired to deliver therapeutic and/or diagnostic molecules (polypeptide and/or nucleotide) into the nucleus of the targeted cell. Any number of crosslinking agents, well known in the art, may be used to covalently link a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence will occur thus facilitating transfer of a plasmid into the nucleus of a targeted cell.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Exemplary nuclear localization sequences include H₂N-GNQSSNFGPMKGGNFGGRSSGPYGGGGQYFAKPRNQGGYGGC-COOH SEQ I.D NO: 9, RRMKWKK (SEQ ID NO:10), PKKKRKV (SEQ ID NO: 11), and KR[PAATKKAGQA]KKKK (SEQ ID NO:12), the NLS of nucleoplasmin, a prototypical bipartite signal comprising two clusters of basic amino acids, separated by a spacer of about 10 amino acids. Numerous other nuclear localization sequences are well known in the art. See, for example, LaCasse, et al., Nuclear localization signals overlap DNA- or RNA-binding domains in nucleic acid-binding proteins, Nucl, Acids Res., 23, 1647-1656 1995); Weis, K. Importins and exportins: how to get in and out of the nucleus [published erratum appears in Trends Biochem Sci 1998 July ; 23(7):235]. TIBS, 23, 185-9 (1998); and Murat Cokol, Raj Nair & Burkhard Rost, “Finding nuclear localization signals”, at the website ubic.bioc.columbia.edu/papers/2000 nls/paper.html # tab2.

The term “cancer” is used to describe a proliferation of tumor cells (neoplasms) having the unique trait of loss of normal controls, resulting in unregulated growth, lack of differentiation, local tissue invasion, and/or metastasis. As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of dysplasia, or loss of differentiation and orientation of cells, and have the properties of invasion and metastasis. The term cancer also within context, includes drug resistant cancers, including multiple drug resistant cancers. Examples of neoplasms or neoplasias from which the target cell of the present disclosure may be derived include, without limitation, carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver (hepatocellular), lung, nasopharyngeal, neck, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' tumor and teratocarcinomas, among others. It is noted that certain tumors including hepatocellular and cervical cancer, among others, are shown to exhibit increased levels of MET receptors specifically on cancer cells and are a principal target for compositions and therapies according to embodiments of the present disclosure which include a MET binding peptide complexed to the protocell.

The terms “coadminister” and “coadministration” are used synonymously to describe the administration of at least one of the protocell compositions according to the present disclosure in combination with at least one other agent, often at least one additional anti-cancer agent (as otherwise described herein), which are specifically disclosed herein in amounts or at concentrations which would be considered to be effective amounts at or about the same time. While it is preferred that coadministered compositions/agents be administered at the same time, agents may be administered at times such that effective concentrations of both (or more) compositions/agents appear in the patient at the same time for at least a brief period of time. Alternatively, in certain aspects of the present disclosure, it may be possible to have each coadministered composition/agent exhibit its inhibitory effect at different times in the patient, with the ultimate result being the inhibition and treatment of cancer, especially including hepatoccellular or cellular cancer as well as the reduction or inhibition of other disease states, conditions or complications. Of course, when more than disease state, infection or other condition is present, the present compounds may be combined with other agents to treat that other infection or disease or condition as required.

The term “anti-cancer agent” is used to describe a compound which can be formulated in combination with one or more compositions comprising protecells according to the present disclosure and optionally, to treat any type of cancer, in particular hepatocellular or cervical cancer, among numerous others. Anti-cancer compounds which can be formulated with compounds according to the present disclosure include, for example, Exemplary anti-cancer agents which may be used in the present disclosure include, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a BcI-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS- 100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901 , AZD-6244, capecitabine, L-Glutamic acid, N-[4-[2-(2-amino-4,7-dihydro-4-oxo-1H pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(But) 6, Azgly 10] (pyro-Glu-His-Trp-Ser-Tyr-D-Ser(Bu t)-Leu-Arg-Pro-Azgly-NH ₂ acetate [C₅₉H₅₄N₁₈Oi₄—(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox,gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

The term “antihepatocellular cancer agent” is used throughout the specification to describe an anticancer agent which may be used to inhibit, treat or reduce the likelihood of hepatocellular cancer, or the metastasis of that cancer. Anticancer agents which may find use in the present disclosure include for example, nexavar (sorafenib), sunitinib, bevacizumab, tarceva (erlotinib), tykerb (lapatinib) and mixtures thereof. In addition, other anticancer agents may also be used in the present disclosure, where such agents are found to inhibit metastasis of cancer, in particular, hepatocellular cancer.

The term “antiviral agent” is used to describe a bioactive agent/drug which inhibits the growth and/or elaboration of a virus, including mutant strains such as drug resistant viral strains. Exemplary antiviral agents include anti-HIV agents, anti-HBV agents and anti-HCV agents. In certain aspects of the disclosure, especially where the treatment of hepatocellular cancer is the object of therapy, the inclusion of an anti-hepatitis C agent or anti-hepatitis B agent may be combined with other traditional anticancer agents to effect therapy, given that hepatitis B virus (HBV) and/or hepatitis C virus (HCV) is often found as a primary or secondary infection or disease state associated with hepatocellular cancer. Anti-HBV agents which may be used in the present disclosure, either as a cargo component in the protocell or as an additional bioactive agent in a pharmaceutical composition which includes a population of protocells includes such agents as Hepsera (adefovir dipivoxil), lamivudine, entecavir, telbivudine, tenofovir, emtricitabine, clevudine, valtoricitabine, amdoxovir, pradefovir, racivir, BAM 205, nitazoxanide, UT 231-B, Bay 41-4109, EHT899, zadaxin (thymosin alpha-1) and mixtures thereof. Typical anti-HCV agents for use in the disclosure include such agents as boceprevir, daclatasvir, asunapavir, INX-189, FV-100, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, GS 9256, GS 9451, GS 5885, GS 6620, GS 9620, GS9669, ACH-1095, ACH-2928, GSK625433, TG4040 (MVA-HCV), A-831, F351, NS5A, NS4B, ANA598, A-689, GNI-104, IDX102, ADX184, ALS-2200, ALS-2158, BI 201335, BI 207127, BIT-225, BIT-8020, GL59728, GL60667, PSI-938, PSI-7977, PSI-7851, SCY-635, pegylated interferon, PHX1766, SP-30 and mixtures thereof.

The term “anti-HIV agent” refers to a compound which inhibits the growth and/or elaboration of HIV virus (I and/or II) or a mutant strain thereof. Exemplary anti-HIV agents for use in the present disclosure which can be included as cargo in protocells according to the present disclosure include, for example, including nucleoside reverse transcriptase inhibitors (NRTI), other non-nucloeoside reverse transcriptase inhibitors (i.e., those which are not representative of the present disclosure), protease inhibitors, fusion inhibitors, among others, exemplary compounds of which may include, for example, 3TC (Lamivudine), AZT (Zidovudine), (-)-FTC, ddl (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof.

The term “targeting active species” is used to describe a compound or moiety which is complexed or for example covalently bonded to the surface of a protocell according to the present disclosure which binds to a moiety on the surface of a cell to be targeted so that the protocell may selectively bind to the surface of the targeted cell and deposit its contents into the cell. The targeting active species for use in the present disclosure is in one embodiment a targeting peptide as otherwise described herein, a polypeptide including an antibody or antibody fragment, an aptamer, or a carbohydrate, among other species which bind to a targeted cell.

The term “targeting peptide” is used to describe an example of a targeting active species which is a peptide of a particular sequence which binds to a receptor or other polypeptide in cancer cells and allows the targeting of protocells according to the present disclosure to particular cells which express a peptide (be it a receptor or other functional polypeptide) to which the targeting peptide binds. In the present disclosure, exemplary targeting peptides include, for example, SP94 free peptide (H₂N-SFSIILTPILPL-COOH, SEQ ID NO: 6), SP94 peptide modified with a C-terminal cysteine for conjugation with a crosslinking agent (H₂N-SFSIILTPILPLGGC-COOH, SEQ ID NO: 7), a modified SP94 peptide (H₂N-SFSIILTPILPLEEEGGC-COOH, SEQ ID NO: 8) or a MET binding peptide as otherwise disclosed herein. Other targeting peptides are known in the art. Targeting peptides may be complexed or covalently linked to the lipid bilayer through use of a crosslinking agent as otherwise described herein.

The term “MET binding peptide” or “MET receptor binding peptide” is used to five (5) 7-mer peptides which have been shown to bind MET receptors on the surface of cancer cells with enhanced binding efficiency. Pursuant to the present disclosure, several small peptides with varying amino acid sequences were identified which bind the MET receptor (a.k.a. hepatocyte growth factor receptor, expressed by gene c-MET) with varying levels of specificity and with varying ability to activate MET receptor signaling pathways. 7-mer peptides were identified using phage display biopanning, with examples of resulting sequences which evidence enhanced binding to MET receptor and consequently to cells such as cancer cells (e.g. hepatocellular, ovarian and cervical) which express high levels of MET receptors, which appear below. Binding data for several of the most commonly observed sequences during the biopanning process is also presented in the examples section of the present application. These peptides are particularly useful as targeting ligands for cell-specific therapeutics. However, peptides with the ability to activate the receptor pathway may have additional therapeutic value themselves or in combination with other therapies. Many of the peptides have been found bind not only hepatocellular carcinoma, which was the original intended target, but also to bind a wide variety of other carcinomas including ovarian and cervical cancer. These peptides are believed to have wide-ranging applicability for targeting or treating a variety of cancers and other physiological problems associated with expression of MET and associated receptors.

The following five 7mer peptide sequences show substantial binding to MET receptor and are particularly useful as targeting peptides for use on protocells according to the the present disclosure.

SEQ ID NO: 1
ASVHFPP (Ala-Ser-Val-His-Phe-Pro-Pro)
SEQ ID NO: 2
TATFWFQ (Thr-Ala-Thr-Phe-Trp-Phe-Gln)
SEQ ID NO: 3
TSPVALL (Thr-Ser-Pro-Val-Ala-Leu-Leu)
SEQ ID NO: 4
IPLKVHP (Ile-Pro-Leu-Lys-Val-His-Pro)
SEQ ID NO: 5
WPRLTNM (Trp-Pro-Arg-Leu-Thr-Asn-Met)

Each of these peptides may be used alone or in combination with other MET peptides within the above group or with other targeting peptides which may assist in binding protocells according to the present disclosure to cancer cells, including hepatocellular cancer cells, ovarian cancer cells and cervical cancer cells, among numerous others. These binding peptides may also be used in pharmaceutical compounds alone as MET binding peptides to treat cancer and otherwise inhibit hepatocyte growth factor binding.

The terms “fusogenic peptide” and “endosomolytic peptide” are used synonymously to describe a peptide which is optionally crosslinked onto the lipid bilayer surface of the protocells according to the present disclosure. Fusogenic peptides are incorporated onto protocells in order to facilitate or assist escape from endosomal bodies and to facilitate the introduction of protocells into targeted cells to effect an intended result (therapeutic and/or diagnostic as otherwise described herein). Representative fusogenic peptides for use in protocells according to the present disclosure include HSWYG peptide, H₂N-GLFHAIAHFIHGGWHGLIHGWYGGC-COOH (SEQ ID. NO: 13) or an 8 mer polyarginine (H₂N-RRRRRRRR-COOH, SEQ ID NO:14), among others known in the art.

The term “crosslinking agent” is used to describe a bifunctional compound of varying length containing two different functional groups which may be used to covalently link various components according to the present disclosure to each other. Crosslinking agents according to the present disclosure may contain two electrophilic groups (to react with nucleophilic groups on peptides of oligonucleotides, one electrophilic group and one nucleophilic group or two two nucleophlic groups). The crosslinking agents may vary in length depending upon the components to be linked and the relative flexibility required. Crosslinking agents are used to anchor targeting and/or fusogenic peptides to the phospholipid bilayer, to link nuclear localization sequences to histone proteins for packaging supercoiled plasmid DNA and in certain instances, to crosslink lipids in the lipid bilayer of the protocells. There are a large number of crosslinking agents which may be used in the present disclosure, many commercially available or available in the literature. Exemplary crosslinking agents for use in the present disclosure include, for example, 1-Ethyl-3-[3-dimethylaminopropyl]carbodiimide hydrochloride (EDC), succinimidyl 4-[N-maleimidomethyl]cyclohexane-1-carboxylate (SMCC), N-[β-Maleimidopropionic acid] hydrazide (BMPH), NHS-(PEG)ₙ-maleimide, succinimidyl-[(N-maleimidopropionamido)-tetracosaethyleneglycol] ester (SM(PEG)₂₄), and succinimidyl 6-[3′-(2-pyridyldithio)-propionamido] hexanoate (LC-SPDP), among others.

As discussed in detail above, the porous nanoparticle core of the present disclosure can include porous nanoparticles having at least one dimension, for example, a width or a diameter of about 3000 nm or less, about 1000 nm or less, about 500 nm or less, about 200 nm or less. For example, the nanoparticle core is spherical with, in certain embodiments, diameter of about 500 nm or less, or about 8-10 nm to about 200 nm. In embodiments, the porous particle core can have various cross-sectional shapes including a circular, rectangular, square, or any other shape. In certain embodiments, the porous particle core can have pores with a mean pore size ranging from about 2 nm to about 30 nm, although the mean pore size and other properties (e.g., porosity of the porous particle core) are not limited in accordance with various embodiments of the present teachings.

In general, protocells according to the present disclosure are biocompatible. Drugs and other cargo components are often loaded by adsorption and/or capillary filling of the pores of the particle core up to approximately 50% by weight of the final protocell (containing all components). In certain embodiments according to the present disclosure, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bilayer(s) as generally described herein.

In the present disclosure, the porous nanoparticle core used to prepare the protocells can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In certain aspects of the disclosure, the lipid bilayer is fused onto the porous particle core to form the protocell. Protocells according to the present disclosure can include various lipids in various weight ratios, including but not limited to 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

The lipid bilayer which is used to prepare protocells according to the present disclosure can be prepared, for example, by extrusion of hydrated lipid films through a filter with pore size of, for example, about 100 nm, using standard protocols known in the art or as otherwise described herein. The filtered lipid bilayer films can then be fused with the porous particle cores, for example, by pipette mixing. In certain embodiments, excess amount of lipid bilayer or lipid bilayer films can be used to form the protocell in order to improve the protocell colloidal stability.

In certain diagnostic embodiments, various dyes or fluorescent (reporter) molecules can be included in the protocell cargo (as expressed by as plasmid DNA) or attached to the porous particle core and/or the lipid bilayer for diagnostic purposes. For example, the porous particle core can be a silica core or the lipid bilayer and can be covalently labeled with FITC (green fluorescence), while the lipid bilayer or the particle core can be covalently labeled with FITC Texas red (red fluorescence). The porous particle core, the lipid bilayer and the formed protocell can then be observed by, for example, confocal fluorescence for use in diagnostic applications. In addition, as discussed herein, plasmid DNA can be used as cargo in protocells according to the present disclosure such that the plasmid may express one or more fluorescent proteins such as fluorescent green protein or fluorescent red protein which may be used in diagnostic applications.

In various embodiments, the protocell is used in a synergistic system where the lipid bilayer fusion or liposome fusion (i.e., on the porous particle core) is loaded and sealed with various cargo components with the pores (mesopores) of the particle core, thus creating a loaded protocell useful for cargo delivery across the cell membrane of the lipid bilayer or through dissolution of the porous nanoparticle, if applicable. In certain embodiments, in addition to fusing a single lipid (e.g., phospholipids) bilayer, multiple bilayers with opposite charges can be successively fused onto the porous particle core to further influence cargo loading and/or sealing as well as the release characteristics of the final protocell

A fusion and synergistic loading mechanism can be included for cargo delivery. For example, cargo can be loaded, encapsulated, or sealed, synergistically through liposome fusion on the porous particles. The cargo can include, for example, small molecule drugs (e.g. especially including anticancer drugs and/or antiviral drugs such as anti-HBV or anti-HCV drugs), peptides, proteins, antibodies, DNA (especially plasmid DNA, including but not limited to histone-packaged super coiled plasmid DNA), RNAs (including shRNA and siRNA (which may also be expressed by the plasmid DNA incorporated as cargo within the protocells) fluorescent dyes, including fluorescent dye peptides which may be expressed by the plasmid DNA incorporated within the protocell.

In embodiments according to the present disclosure, the cargo can be loaded into the pores (mesopores) of the porous particle cores to form the loaded protocell. In various embodiments, any conventional technology that js developed for liposome-based drug delivery, for example, targeted delivery using PEGylation, can be transferred and applied to the the protocells of the present disclosure.

As discussed above, electrostatics and pore size can play a role in cargo loading. For example, porous silica nanoparticles can carry a negative charge and the pore size can be tunable from about 2 nm to about 10 nm or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively species can be loaded as cargo into the pores of a negatively charged silica particle when the lipid bilayer is fused onto the silica surface showing a fusion and synergistic loading mechanism. In this manner, fusion of a non-negatively charged (i.e., positively charged or neutral) lipid bilayer or liposome on a negatively charged mesoporous particle can serve to load the particle core with negatively charged cargo components. The negatively charged cargo components can be concentrated in the loaded protocell having a concentration exceed about 100 times as compared with the charged cargo components in a solution. In other embodiments, by varying the charge of the mesoporous particle and the lipid bilayer, positively charged cargo components can be readily loaded into protocells.

Once produced, the loaded protocells can have a cellular uptake for cargo delivery into a desirable site after administration. For example, the cargo-loaded protocells can be administered to a patient or subject and the protocell comprising a targeting peptide can bind to a target cell and be internalized or uptaken by the target cell, for example, a cancer cell in a subject or patient. Due to the internalization of the cargo-loaded protocells in the target cell, cargo components can then be delivered into the target cells. In certain embodiments the cargo is a small molecule, which can be delivered directly into the target cell for therapy. In other embodiments, negatively charged DNA or RNA (including shRNA or siRNA), especially including a DNA plasmid which may be formulated as histone-packaged supercoiled plasmid DNA, e.g., modified with a nuclear localization sequence, can be directly delivered or internalized by the targeted cells. Thus, the DNA or RNA can be loaded first into a protocell and then into then through the target cells through the internalization of the loaded protocells.

As discussed, the cargo loaded into and delivered by the protocell to targeted cells includes small molecules or drugs (especially anti-cancer or anti-HBV and/or anti-HCV agents), bioactive macromolecules (bioactive polypeptides such as ricin toxin A-chain or diphtheria toxin A-chain or RNA molecules such as shRNA and/or siRNA as otherwise described herein) or histone-packaged supercoiled plasmid DNA which can express a therapeutic or diagnostic peptide or a therapeutic RNA molecule such as shRNA or siRNA, wherein the histone-packaged supercoiled plasmid DNA is optionally and in one embodiment modified with a nuclear localization sequence which can localize and concentrate the delivered plasmid DNA into the nucleus of the target cell. As such, loaded protocells can deliver their cargo into targeted cells for therapy or diagnostics.

In various embodiments according to the present disclosure, the protocells and/or the loaded protocells can provide a targeted delivery methodology for selectively delivering the protocells or the cargo components to targeted cells (e.g., cancer cells). For example, a surface of the lipid bilayer can be modified by a targeting active species that corresponds to the targeted cell. The targeting active species may be a targeting peptide as otherwise described herein, a polypeptide including an antibody or antibody fragment, an aptamer, a carbohydrate or other moiety which binds to a targeted cell. In certaom aspects of the disclosure, the targeting active species is a targeting peptide as otherwise described herein. In certain embodiments, an exemplary peptide targeting species include a MET binding peptide as otherwise described herein.

For example, by providing a targeting active species (e.g., a targeting peptide) on the surface of the loaded protocell, the protocell selectively binds to the targeted cell in accordance with the present teachings. In one embodiment, by conjugating an exemplary targeting peptide SP94 or analog or a MET binding peptide as otherwise described herein that targets cancer cells, including cancer liver cells to the lipid bilayer, a large number of the cargo-loaded protocells can be recognized and internalized by this specific cancer cells due to the specific targeting of the exemplary SP94 or MET binding peptide with the cancer (including liver) cells. In most instances, if the protocells are conjugated with the targeting peptide, the protocells will selectively bind to the cancer cells and no appreciable binding to the non-cancerous cells occurs.

Once bound and taken up by the target cells, the loaded protocells can release cargo components from the porous particle and transport the released cargo components into the target cell. For example, sealed within the protocell by the liposome fused bilayer on the porous particle core, the cargo components can be released from the pores of the lipid bilayer, transported across the protocell membrane of the lipid bilayer and delivered within the targeted cell. In embodiments according to the present disclosure, the release profile of cargo components in protocells can be more controllable as compared with when only using liposomes as known in the prior art. The cargo release can be determined by, for example, interactions between the porous core and the lipid bilayer and/or other parameters such as pH value of the system. For example, the release of cargo can be achieved through the lipid bilayer, through dissolution of the porous silica; while the release of the cargo from the protocells can be pH-dependent.

In certain embodiments, the pH value for cargo is often less than 7, e.g., about 4.5 to about 6,0, but can be about pH 14 or less. Lower pHs tend to facilitate the release of the cargo components significantly more than compared with high pHs. Lower pHs tend to be advantageous because the endosomal compartments inside most cells are at low pHs (about 5.5), but the rate of delivery of cargo at the cell can be influenced by the pH of the cargo. Depending upon the cargo and the pH at which the cargo is released from the protocell, the release of cargo can be relative short (a few hours to a day or so) or a span for several days to about 20-30 days or longer. Thus, the present disclosure may accommodate immediate release and/or sustained release applications from the protocells themselves.

In certain embodiments, the inclusion of surfactants can be provided to rapidly rupture the lipid bilayer, transporting the cargo components across the lipid bilayer of the protocell as well as the targeted cell. In certain embodiments, the phospholipid bilayer of the protocells can be ruptured by the application/release of a surfactant such as sodium dodecyl sulfate (SDS), among others to facilitate a rapid release of cargo from the protocell into the targeted cell. In certain embodiments, the rupture of the lipid bilayer can in turn induce immediate and complete release of the cargo components from the pores of the particle core of the protocells. In this manner, the protocell platform can provide versatile delivery systems as compared with other delivery systems in the art. For example, when compared to delivery systems using nanoparticles only, the disclosed protocell platform can provide a simple system and can take advantage of the low toxicity and immunogenicity of liposomes or lipid bilayers along with their ability to be PEGylated or to be conjugated to extend circulation time and effect targeting. In another example, when compared to delivery systems using liposome only, the protocell platform can provide a more stable system and can take advantage of the mesoporous core to control the loading and/or release profile.

In addition, the lipid bilayer and its fusion on porous particle core can be fine-tuned to control the loading, release, and targeting profiles and can further comprise fusogenic peptides and related peptides to facilitate delivery of the protocells for greater therapeutic and/or diagnostic effect. Further, the lipid bilayer of the protocells can provide a fluidic interface for ligand display and multivalent targeting, which allows specific targeting with relatively low surface ligand density due to the capability of ligand reorganization on the fluidic lipid interface. Furthermore, the disclosed protocells can readily enter targeted cells while empty liposomes without the support of porous particles cannot be internalized by the cells.

Pharmaceutical compositions according to the present disclosure comprise an effective population of protocells as otherwise described herein formulated to effect an intended result (e.g. therapeutic result and/or diagnostic analysis, including the monitoring of therapy) formulated in combination with a pharmaceutically acceptable carrier, additive or excipient. The protocells within the population of the composition may be the same or different depending upon the desired result to be obtained. Pharmaceutical compositions according to the present disclosure may also comprise an addition bioactive agent or drug, such as an anticancer agent or an antiviral agent, for example, an anti-HIV, anti-HBV or an anti-HCV agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g. orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method of the disclosure include humans, companion animals, laboratory animals, and the like. The disclosure contemplates immediate and/or sustained/controlled release compositions, including compositions which comprise both immediate and sustained release formulations. This is particularly true when different populations of protocells are used in the pharmaceutical compositions or when additional bioactive agent(s) are used in combination with one or more populations of protocells as otherwise described herein.

Formulations containing the compounds according to the present disclosure may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, e.g.,in unit dosage forms suitable for simple administration of precise dosages.

Pharmaceutical compositions according to the present disclosure typically include a conventional pharmaceutical carrier or excipient and may additionally include other medicinal agents, carriers, adjuvants, additives and the like. For instance, the composition is about 0.1% to about 85%, about 0.5% to about 75% by weight of a compound or compounds of the disclosure, with the remainder consisting essentially of suitable pharmaceutical excipients.

An injectable composition for parenteral administration (e.g. intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of protoells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

Methods for preparing such dosage forms are known or is apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject according to the present disclosure.

Methods of treating patients or subjects in need for a particular disease state or infection (especially including cancer and/or a HBV, HCV or HIV infection) comprise administration an effective amount of a pharmaceutical composition comprising therapeutic protocells and optionally at least one additional bioactive (e.g. antiviral) agent according to the present disclosure.

Diagnostic methods according to the present disclosure comprise administering to a patient in need (a patient suspected of having cancer) an effective amount of a population of diagnostic protocells (e.g., protocells which comprise a target species, such as a targeting peptide which binds selectively to cancer cells and a reporter component to indicate the binding of the protocells to cancer cells if the cancer cells are present) whereupon the binding of protocells to cancer cells as evidenced by the reporter component (moiety) will enable a diagnosis of the existence of cancer in the patient.

An alternative of the diagnostic method of the present disclosure can be used to monitor the therapy of cancer or other disease state in a patient, the method comprising administering an effective population of diagnostic protocells (e.g., protocells which comprise a target species, such as a targeting peptide which binds selectively to cancer cells or other target cells and a reporter component to indicate the binding of the protocells to cancer cells if the cancer cells are present) to a patient or subject prior to treatment, determining the level of binding of diagnostic protocells to target cells in said patient and during and/or after therapy, determining the level of binding of diagnostic protocells to target cells in said patient, whereupon the difference in binding before the start of therapy in the patient and during and/or after therapy will evidence the effectiveness of therapy in the patient, including whether the patient has completed therapy or whether the disease state has been inhibited or eliminated (including remission of a cancer).

### Exemplary Embodiments

In one embodiment, a porous protocell is provided comprising: a nanoporous silica or metal oxide core with a supported lipid bilayer and one or more non-integrating vectors comprising nucleic acid encoding a non-nucleolytic fusion protein comprising a protein sequence that specifically binds a sequence in a double strand nucleic acid molecule linked to a protein sequence that is a transcriptional mediator, and optionally guide RNA or a vector for expression of guide RNA. In one embodiment, at least one of the vectors is a non-integrating plasmid. In one embodiment, the transcriptional mediator is a transcriptional activator. In one embodiment, the transcriptional mediator is a transcriptional repressor. In one embodiment, the protein sequence that specifically binds a sequence in a double strand nucleic acid molecule is N-terminal to the protein sequence that is a transcriptional mediator. In one embodiment, the protein sequence that specifically binds a sequence in a double strand nucleic acid molecule is C-terminal to the protein sequence that is a transcriptional mediator. In one embodiment, the vector encoding the fusion protein encodes guide RNA. In one embodiment, the protein sequence that specifically binds a double strand nucleic acid molecule is a Cas protein. In one embodiment, the protein sequence that is a transcriptional mediator comprises VP64. In one embodiment, the gRNA is sgRNA comprising a base-pairing sequence, a dCas9-binding hairpin and a terminator. In one embodiment, the protocell further comprises a nucleic acid encoding an activator helper protein. In one embodiment, the helper protein comprises MS2-P65-HSF1. In one embodiment, the nucleic acid encoding an activator helper protein is on a different vector than the vector comprising the nucleic acid encoding the fusion protein. In one embodiment, the gRNA is for p53, HIF, apelin, ANGPTL4, FGF21, or FNDC5. In one embodiment, the transcription activator comprises VP16, TA2, VP64 (a tetrameric repeat of the minimal activation domain of VP16), signal transducer and activator of transcription 6 (STATE), reticuloendotheliosis virus A oncogene (relA), TATA binding protein associated factor-1 (TAF-1), TATA binding protein associated factor-2 (TAF-2), glucocorticoid receptor TAU-1, or glucocorticoid receptor TAU-2, or a portion thereof having transcription activating activity. In one embodiment, the transcription repressor comprises ETS repressor factor, the ETS repressor factor repressor domain (ERD), Kruppel-associated box (KRAB), human MAD1 protein, mSin3 interaction domain of the human MAD1 protein (SD), histone deacetylase, DNA methylase, or is a derivative or multimer of KRAB, SD, or ERD selected from the group consisting of KRAB-ERD, SID-ERD, (KRAB)₂, (KRAB)₃, KRAB-A, (KRAB- A)₂, (SID)₂, (KRAB-A)-SID, or SID-KRAB-A, or a portion thereof having transcription repressing activity. In one embodiment, the protocell comprises two or more different unlinked vectors. In one embodiment, the protocell comprises two or more different plasmids. In one embodiment, the protein sequence that specifically binds the sequence comprises a transcription factor, Gal4, hypoxia inducible factor (HIF), e.g., HIF1α, cyclic AMP response element binding (CREB) protein, LexA, rtTA, an endonuclease, a zinc finger binding domain, or a synthetic DNA binding domains. In one embodiment, the protein sequence that specifically binds DNA comprises a RNA-guided DNA endonuclease that lacks endonuclease activity. In one embodiment, the protocell further comprises at least one component selected from the group consisting of a cell targeting species; a fusogenic peptide that promotes endosomal escape of protocells and encapsulated DNA, and at least one cargo component selected from the group consisting of double stranded linear DNA; a drug; an imaging agent, small interfering RNA, small hairpin RNA, micro RNA, or a mixture thereof, wherein one of said cargo components is optionally conjugated further with a nuclear localization sequence. In one embodiment, said silica core is spherical and ranges in diameter from about 10 nm to about 250 nm. In one embodiment, said silica core has a mean diameter of about 150 nm. In one embodiment, said silica core is monodisperse or polydisperse in size distribution. In one embodiment, said lipid bilayer is comprised of lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures thereof. In one embodiment, said lipid bilayer comprises DOTAP in combination with DOPE. In one embodiment, said lipid bilayer comprises DOTAP, DOPG, DOPC, DOPE, or mixtures thereof. In one embodiment, said lipid bilayer comprises PEGylated lipid. In one embodiment, said lipid bilayer comprises about 55% by weight DOTAP, about 5 wt % DOPE, about 30 wt % cholesterol, and about 10 wt % PEG-2000 PE. In one embodiment, the lipid bilayer comprises about 55% by weight DOTAP, about 5% by weight DOPE, about 30% by weight cholesterol, and about 10% by weight DSPE-PEG 2000. In one embodiment, wherein the lipid bilayer comprises DOTAP. In one embodiment, said targeting species is a targeting peptide. In one embodiment, at least one of the one or more vectors comprises an operably linked nuclear localization sequence. In one embodiment, the protocell father comprises an anticancer agent. In one embodiment, said anticancer agent is everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a BcI-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAKISTAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601 , ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, 5′-deoxy-5-fluorouridine, vincristine, temozolomide, ZK-304709, seliciclib; PD0325901 , AZD-6244, capecitabine, L-Glutamic acid, N-[4-[2-(2-amino-4,7-dihydro-4-oxo-1H-pyrrolo[2,3- d]pyrimidin-5-yl)ethyl]benzoyl], disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)- indolylj-quinolone, vatalanib, AG-013736, AVE-0005, the acetate salt of [D-Ser(Bu t) 6,Azgly 10] (pyro-Glu-His-Trp-Ser-Tyr-D-Ser(Bu t)-Leu-Arg-Pro- Azgly-NH ₂ acetate [C₅₉H₈₄N₁₈Oi₄—(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, BMS-214662, tipifarnib; amifostine_{;} NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimetidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa or a mixture thereof. In one embodiment, said drug comprises an antiviral agent. In one embodiment, said antiviral agent is an anti-HIV agent, an anti-HBV agent or an anti-HCV agent.

In one embodiment, a pharmaceutical composition comprising a population of protocells in an amount effective for effecting a therapeutic effect in combination with a pharmaceutically acceptable carrier, additive or excipient is provided. In one embodiment, the composition is in parenteral dosage form. In one embodiment, said dosage form is intradermal, intramuscular, intraosseous, intraperitoneal, intravenous, subcutaneous or intrathecal. In one embodiment, the composition is in topical or transdermal dosage form.

In one embodiment, a method of treating cancer is provided. The method includes administering to a patient in need an effective amount of a composition comprising a population of protocells as described above which have been adapted to deliver an anticancer agent to a cancer cell in said patient.

In one embodiment, a method of diagnosing cancer in a patient at risk for cancer is provided. In one embodiment, the method includes administering to said patient a pharmaceutical composition comprising a population of protocells as described herein comprising targeting peptides adapted to selectively bind to cancer cells and deliver the protocells to said cells, wherein said protocells comprise a plasmid DNA adapted to express a reporter molecule and optionally comprise an additional reporter molecule, whereupon the binding of the protocell to a cancer cell in said patient will release said reporter molecules into the cancer cells, if present, and the reporter molecules will elicit a signal which can be compared with a standard to determine whether or not the patient has cancer and if so, the extent of the cancer and/or size of a cancerous tumor, if present.

In one embodiment, a method of monitoring cancer therapy in a patient is provided. The method includes administering to said patient a population of protocells as described herein targeting peptides adapted to selectively bind to cancer cells and deliver the protocells to said cells, wherein said protocells comprise a plasmid DNA adapted to express a reporter molecule and optionally comprise an additional reporter molecule, whereupon the binding of the protocell to a cancer cell in said patient will release said reporter molecules into the cancer cells and the reporter molecules will elicit a signal which can be compared with a standard at the commencement of therapy and at varying intervals during the course of therapy to determine whether or not patient is responding to the therapy and if so, the extent of the response to the therapy.

In one embodiment, a method to enhance a response to hypoxia in a mammal is provided. The method includes administering to the mammal an effective amount of a composition comprising a population of protocells as described herein, wherein the protocells comprise a guide RNA or a vector for expression of guide RNA specific for a gene that is upregulated in response to hypoxia.

In one embodiment, a method to enhance a response to excessive cold in a mammal is provided. The method includes administering to the mammal an effective amount of a composition comprising a population of protocells according to any of claims 1 to 36, wherein the protocells comprise a guide RNA or a vector for expression of guide RNA specific for a gene that is upregulated in response to cold stress.

The invention will be further described by the following non-limiting examples.

### Example

As provided in the following examples, the porous nanoparticle-supported lipid bilayer (protocell), formed via fusion of liposomes to nanoporous silica particles, is a novel type of nanocarrier that addresses multiple challenges associated with targeted delivery of cancer therapeutics and diagnostics. Like liposomes, protocells are biocompatible, biodegradable, and non-immunogenic, but their nanoporous silica core confers a drastically enhanced cargo capacity and prolonged bilayer stability when compared to similarly-sized liposomal delivery agents. The porosity and surface chemistry of the core can, furthermore, be modulated to promote encapsulation of a wide variety of therapeutic agents, such as drugs, nucleic acids, and protein toxins. The rate of cargo release can be controlled by pore size and the overall degree of silica condensation, making protocells useful in applications requiring either burst or controlled release profiles. Finally, the protocell's supported lipid bilayer (SLB) can be modified with ligands to promote selective delivery and with PEG to extend circulation times. In the examples, the inventors report the use of peptidetargeted protocells to achieve highly specific delivery of a plasmid that encodes small hairpin RNA (shRNA), which induces growth arrest and apoptosis of transfected cells by silencing cyclin B1. As set forth in the examples section below, the inventors have prepared synthesized silica nanoparticles with pores large enough to accommodate histone-packaged plasmids using a dual surfactant approach. A non-ionic surfactant (Pluronic® F-127), when employed in conjunction with a swelling agent (1,3,5-trimethylbenzene) served as the template for large pores, while a fluorocarbon surfactant (FC-4) promoted growth of the silica core, Resulting particles had diameters ranging from 100-nm to 300-nm and contained an ordered network of 20-nm pores with 17.3-nm pore entrances. Supercoiled plasmid DNA was packaged with histones, and the resulting complex (about 15-nm in diameter) was modified with a nuclear localization sequence (NLS) prior to being loaded into the silica core. Fusion of liposomes to the nanoporous core promoted long-term retention (>1 month) of encapsulated DNA upon exposure to simulated body fluids at 37° C. Using phage display, the inventors identified a targeting peptide with nano molar affinity for hepatocyte growth factor receptor (c-Met), which is known to be overexpressed by various types of hepatocellular carcinoma (HCC). Protocells loaded with the DNA-histone-NLS complex and modified with “240 copies each of the targeting peptide and a fusogenic peptide that promotes endosomal escape of protocells and encapsulated DNA were capable of transfecting both dividing and non-dividing HCC. Furthermore, targeted protocells effectively induced GJM arrest and apoptosis of HCC (LC=25 nM) without affecting the viability of non-cancerous cells, including hepatocytes, endothelial cells, and immune cells (PBMCs, B cells, and T cells).

### Methods

The nanoporous silica particles that form the core of the protocell are prepared, as previously described^{1,2} (see also Ashley, et al., Nature Materials, 2011, May; 10(5):389-97) from a homogenous mixture of water-soluble silica precursor(s) and amphipathic surfactant(s) using either aerosol-assisted evaporation-induced self-assembly (EISA) or solvent extraction-driven self-assembly within water-in-oil emulsion droplets¹. Solvent evaporation or extraction concentrates the aerosol or emulsion droplets in surfactant(s), which directs the formation of periodic, ordered structures, around which silica assembles and condenses. Surfactants are removed via thermal calcination, which results in porous nanoparticles with well-defined, uniform pore sizes and topologies. Particles formed via aerosol-assisted EISA (‘unimodal’ particles) possess an average diameter of approximately 120-nm (after size exclusion-based separation), a Brunauer-Emmer-Teller (BET) surface area in excess of 1200 m²/g, a pore volume fraction of about 50%, and a unimodal pore diameter of 2.5-nm. Particles formed within emulsion droplets (‘multimodal’ particles) have an average diameter of ˜150 nm (after size exclusion-based separation), a BET surface area of >600 m²/g_{;} a pore volume fraction of ˜65%, and a multimodal pore morphology composed of large (20-30 nm), surface-accessible pores interconnected by 6-12 nm pores. The liquid-vapor or liquid-liquid interfacial tensions associated with aerosol or emulsion processing (respectively) enforce a spherical shape with minimal surface roughness. Both types of particles, additionally, have fully accessible three-dimensional pore networks, as evidenced by analysis of nitrogen sorption isotherms.

The high pore volume, surface area, and accessibility of the nanoporous silica cores imparts a high cargo capacity and enables rapid loading of multiple types of therapeutic and diagnostic agents. Unimodal nanoporous cores have a high capacity for low molecular weight chemotherapeutic agents, while multimodal cores possess the large, surface-accessible pores necessary for encapsulation of siRNA, protein toxins, and other high molecular weight cargos (e.g. plasmid DNA). The rate of cargo release can be precisely controlled by the degree to which the silica core is condensed. Incorporating various amounts of AEPTMS, an amine-containing silane, into the sol used to form the nanoporous silica cores reduces the level of achievable condensation and promotes more rapid dissolution of the cores under neutral pH, high ionic strength (i.e. cytosolic) conditions. Particles that contain no AEPTMS dissolve over the course of 2 weeks in a simulated body fluid, while particles that contain 30 mol % AEPTMS dissolve within 24 hours. Protocells can, therefore, be adapted for applications requiring continuous or burst release profiles.

Incorporating AEPTMS into the precursor sol used to form nanoporous silica particles accelerates particle dissolution under cytosolic conditions and promotes more rapid release of encapsulated cargo than can be achieved via simple diffusion. AEPTMS-modified particles also have a reduced capacity for weakly basic chemotherapeutic drugs (e.g. doxorubicin), however. Therefore, in order to maximize both capacity and intracellular release, we characterized zeta potential, cargo (e.g. drug (Doxorubicin/DOX)/chemotherapy) capacity, silica dissolution rates, and cargo release rates as a function of AEPTMS concentration. As previously demonstrated, unmodified unimodal particles (ζ=−104.5±5.6) have a high capacity for cargo (in the case of DOX about 1.8 mM per 10¹⁰ particles) but release only 20% of their encapsulated cargo (drug) within 24 hours (i.e. the typical doubling time of HCC). Conversely, unimodal particles modified with 30 wt % AEPTMS (ζ=88.9±5.5) release all of their encapsulated cargo (drug) within 6 hours but have a reduced drug (DOX) capacity (˜0.15 mM per 10¹⁰ particles). Unimodal particles that contain 15 wt % AEPTMS (ζ=−21.3±5.1) retain their high capacity for drug (DOX) (˜1.1 mM per 10¹⁰ particles) and release nearly all of their encapsulated (drug) within 24 hours when exposed to a simulated body fluid; therefore these particles are selected for all experiments involving delivery of cargo. It is important to note that, while the zeta potential of unimodal silica particles increases as a function of AEPTMS concentration, the pore volume fraction of AEPTMS-modified particles (˜45% for particles that contain 30 wt % AEPTMS) is not substantially different from that of unmodified particles (˜50%). Therefore, we attribute the decreased cargo capacity of AEPTMS-modified unimodal particles to electrostatic repulsion rather than decreased pore volume. Multimodal particles are included as a control to demonstrate the effect of pore size on cargo capacity and the kinetics of cargo release.

### General Reagents

Absolute ethanol, hydrochloric acid (37%), tetraethyl orthosilicate (TEOS, 98%), 3-aminopropyltriethoxysilane (APTES, ≥98%), 3-[2-(2-aminoethylamino)ethylamino]propyltrimethosilane (AEPTMS, technical grade), 2-cyanoethyl triethoxysilane (CETES, ≥97.0%), hexadecyltrimethylammonium bromide (CTAB, ≥99%), Brij®-56, sodium dodecyl sulfate (SDS, ≥98.5%, Triton® X-100, hexadecane (≥99%), doxorubicin hydrochloride (≥98%), 5-fluorouracil (≥99%), cis-diammineplatinum(II) dichloride (cisplatin, ≥99.9%), diphtheria toxin from Corynebacterium diphtheriae, cyclosporin A from Tolypocladium inflatum (CsA, ≥95%), N-Acetyl-L-cysteine (NAC, ≥99%), human epidermal growth factor, L-α-phosphatidylethanolamine, thymidine (≥99%), hypoxanthine (≥99%), bovine fibronectin, bovine collagen type I, gelatin, soybean trypsin inhibitor (98%), 2-mercaptoethanol (≥99.0%), DL-dithiothreitol (≥99.5%), dimethyl sulfoxide (≥99.9%), pH 5 citric acid buffer, ethylenediaminetetraacetic acid (EDTA, 99.995%), 4-(2-Hydroxyethyl)piperazine-1-ethanesulfonic acid (HEPES, ≥99.5%), ammonium phosphate dibasic (≥99.99%), and Sepharose® CL-4B were purchased from Sigma-Aldrich (St. Louis, Mo.). ABIL® EM 90 (cetyl PEG/PPG-10/1 dimethicone) was purchased from Evonik Industries (Essen, Germany). Ultra pure, EM-grade formaldehyde (16%, methanol-free) was purchased from Polysciences, Inc. (Warrington, Pa.). Hellmanex® II was purchased from Hellma (Müllheim, Germany).

### Lipids

1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-3-trimethylammonimn-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl]amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and cholesterol were purchased from Avanti Polar Lipids, Inc. (Alabaster, Ala.).

### Cell Lines and Growth Media

Human Hep3B (HB-8064), human hepatocytes (CRL-11233), human peripheral blood mononuclear cells (CRL-9855), human umbilical cord vein endothelial cells (CRL-2873), T lymphocytes (CRL-8293), B lymphocytes (CCL-156), Eagle's Minimum Essential Medium (EMEM), Dulbecco's Modified Eagle's Medium (DMEM), Iscove's Modified Dulbecco's Medium (IMDM), RPMI 1640 medium, fetal bovine serum (FBS), and 1× trypsin-EDTA solution (0.25% trypsin with 0.53 mM EDTA) were purchased from American Type Culture Collection (ATCC; Manassas, Va.). BEGM Bullet Kits were purchased from Lonza Group Limited (Clonetics; Walkersville, Md.). DMEM without phenol red was purchased from Sigma-Aldrich (St. Louis, Mo.).

### Fluorescent Stains and Microscopy Reagents

Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-ir AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580), Alexa Fluor® 532 carboxylic acid, succinimidyl ester (532/554), propidium iodide (535/617), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670), Alexa Fluor® 647 conjugate of annexin V (650/665), SlowFade® Gold antifade reagent (with and without DAPI), Image-ir FX signal enhancer, 1× Dulbecco's phosphate-buffered saline (D-PBS), bovine albumin fraction V solution (BSA, 7.5%), and transferrin were purchased from Invitrogen Life Sciences (Carlsbad, Calif.). Red Fluorescent Protein (RFP, 557/585), CaspGLOW™ Fluorescein Active Caspase-3 Staining Kit (485/535), and CaspGLOW™ Red Active Caspase-8 Staining Kit (540/570) were purchased from BioVision, Inc. (Mountain View, Calif.). Water soluble CdSe/ZnS quantum dots, CZWD640 (640/660), were purchased from NN-Labs (Fayetteville, Ariz.).

### Crosslinkers

1-Ethyl-3[3-dimethylaminopropyl]carbodiimide hydrochloride (EDC), succinimidyl 4-[N-maleimidomethyl]cyclohexane-1-carboxylate (SMCC), N-[β-Maleimidopropionic acid] hydrazide (BMPH), succinimidyl-[(N-maleimidopropionamido)-tetracosaethyleneglycol] ester (SM(PEG)₂₄), succinimidyl 6-[3′-(2-pyridyldithio)-propionamido] hexanoate (LC-SPDP), and the Sulfhydryl Addition Kit were purchased from Pierce Protein Research Products (Thermo Fisher Scientific LSR; Rockford, Ill.).

### Other Silica Nanoparticles

Sub-5-nm silicon nanoparticles were purchased from Melorium Technologies, Inc. (Rochester, N.Y.). 10-20 nm silicon oxide nanoparticles were purchased from SkySpring Nanomaterials, Inc. (Houston, Tex.). 30-nm, 40-nm, 50-nm, 60-nm, 70-nm, 80-nm, 90-nm, 100-nm, 150-nm, 200-nm, and 10-μm silica particles were purchased from Discovery Scientific, Inc. (Vancouver, British Columbia).

### Synthetic siRNA and Peptides

Silencer select siRNAs (siRNA IDs for EGFR, VEGFR-2, and PDGFR-α are s565, s7824, and s10234, respectively) were purchased from Ambion, Inc. (Austin, Tex.). The double stranded-DNA oligonucleotide (5′-AAACATGTGGATTACCCATGTC-3′) with 5′ amino modifier C12 was purchased from Integrated DNA Technologies (IDT; Coralville, Iowa). ‘Free’ SP94 peptide (H₂N-SFSIILTPILPL-COOH, SEQ ID NO: 6), SP94 peptide modified with C-terminal Cys for conjugation (H₂N-SFSIILTPILPLGGC-COOH, SEQ ID NO: 7), and SP94 peptide used in the recruitment experiments (H₂N-SFSIILTPILPLEEEGGC-COOH, SEQ ID NO: 8) were synthesized by New England Peptide (Gardner, Mass.). The H5WYG peptide (H₂N-GLFHAIAHFIHGGWHGLIHGWYGGGC-COOH, SEQ ID NO:15) and nuclear localization sequence (H2N-NQSSNFGPMKGGNFGGRSSGPYGGGGQYFAKPRNQGGYGGC-COOH, SEQ ID NO:16) were synthesized by Biopeptide Co., Inc. (San Diego, Calif.). The emboldened portions of peptides are the original sequences; additional amino acid residues were added for conjugation or labeling purposes. All antibodies (CHALV-1, anti-Rab11a, anti-LAMP-1, anti-EGFR, anti-VEGFR-2, anti-PDGFR-α) were purchased from Abcam, Inc. (Cambridge, Mass.).

### Cell Culture Conditions

Hep3B, hepatocytes, PBMCs, T-lymphocytes, and B-lymphocytes were obtained from ATCC and grown per manufacturers instructions. Briefly, Hep3B was maintained in EMEM with 10% FBS. Hepatocytes were grown in flasks coated with BSA, fibronectin, and bovine collagen type I; the culture medium used was BEGM (gentamycin, amphotericin, and epinephrine were discarded from the BEGM Bullet kit) with 5 ng/mL epidermal growth factor, 70 ng/mL phosphatidylethanolamine, and 10% FBS. HUVECs were grown in DMEM with 20% FBS; gelatin-coated flasks were used to promote adhesion. PBMCs, T lymphocytes, and B lymphocytes were maintained in suspension flasks (Greiner Bio-One; Monroe, N.C.). PBMCs were grown in IMDM supplemented with 0.02 mM thymidine, 0.1 mM hypoxanthine, 0.05 mM 2-mercaptoethanol, and 10% FBS. T and B lymphocytes were grown in IMDM with 20% FBS and RPM 1640 medium with 20° i FBS, respectively. All cells were maintained at 37° C. in a humidified atmosphere (air supplemented with 5% CO₂). Adherent cells were passaged with 0.05% trypsin at a sub-cultivation ratio of 1:3, while non-adherent cells were seeded at a density of 2×10⁵ cells/mL and maintained at 1-5×10⁶ cells/mL.

### Synthesis and Characterization of Nanoporous Silica Particles

### Synthesis of Unimodal Silica Nanoparticles

The aerosol-assisted evaporation-induced self-assembly method employed to prepare nanoporous silica particles with unimodal porosity has been described by Lu, et al.². Briefly, a homogenous sol containing a silica precursor (TEOS), a structure-directing surfactant (CTAB, initially at a concentration much less than the critical micelle concentration, or CMC), and HCl dissolved in a solution of water and ethanol was aerosolized using a modified commercial atomizer (Model 9302A; TSI, Inc.; St Paul, Minn.). Nitrogen was used as the carrier gas, and all heating zones were maintained at 400° C. to evaporate the solvent and increase the effective surfactant concentration. Pressure drop at the pinhole was 20 psi. Particles were collected on a Durapore membrane fitter (Millipore; Billerica, Mass.) maintained at 80° C. A typical reaction mixture contained 55.9 mL of deionized H₂O, 43 mL of 200-proof ethanol, 1.10 mL of 1.0 N HCl, 4.0 g of CTAB, and 10.32 g of TEOS. To prepare nanoporous silica particles that dissolve more rapidly under intracellular (neutral pH, relatively high salt concentrations) conditions, various amounts of TEOS and AEPTMS, an amine-containing silane, were incorporated into the precursor sol, and the pH of the system was adjusted to 2.0 using concentrated HCl. For example, to prepare particles with 15 wt % AEPTMS, 9.36 g of TEOS and 1.33 g of AEPTMS were used.

### Synthesis of Multimodal Silica Nanoparticles

The emulsion processing used to synthesize nanoporous silica particles with multimodal porosity has been described by Carroll, et al.'. Briefly, 1.82 g of CTAB (soluble in the aqueous phase) was added to 20 g of deionized water, stirred at 40° C. until dissolved, and allowed to cool to 25° C. 0.57 g of 1.0 N HCl, 5.2 g of TEOS, and 0.22 g of NaCl were added to the CTAB solution, and the resulting sol was stirred for 1 hour. An oil phase composed of hexadecane with 3 wt % Abil EM 90 (a non-ionic emulsifier soluble in the oil phase) was prepared. The precursor sol was combined with the oil phase (1:3 volumetric ratio of sol:oil) in a 1000-mL round-bottom flask, stirred vigorously for 2 minutes to promote formation of a water-in-oil emulsion, affixed to a rotary evaporator (R-205; Buchi Laboratory Equipment; Switzerland), and placed in an 80° C. water bath for 30 minutes. The mixture was then boiled under a reduced pressure of 120 mbar (35 rpm for 3 hours) to remove the solvent. Particles were the centrifuged (Model Centra MP4R; International Equipment Company; Chattanooga, Tenn.) at 3000 rpm for 20 minutes, and the supernatant was decanted. Finally, the particles were calcined at 500° C. for 5 hours to remove surfactants and other excess organic matter. As described by Carroll, etal., solvent extraction enriches the aqueous phase in CTAB (>CMC), and the resulting micelles template 6-12 nm pores upon condensation of silica particles (in the aqueous phase). Additionally, adsorption of two surfactants (CTAB and Abil EM 90) at the water-oil interface synergistically decreases the interfacial tension, which results in the spontaneous formation of 20-30 nm microemulsion droplets that template large, surface-accessible pores.

### Characterization of Silica Nanoparticles

Dynamic light scattering of nanoporous silica particles was performed using a Zetasizer Nano (Malvern; Worcestershire, United Kingdom). Samples were prepared by diluting 48 μL of silica particles (25 mg/mL) in 2.4 ml of 1×D-PBS. Solutions were transferred to 1 mL polystyrene cuvettes (Saistedt; Numbrecht, Germany) for analysis. Nitrogen sorption was performed using an ASAP 2020 Surface Area and Porosity Analyzer (Micromeritics Instrument Corporation; Norcross, Ga.). Zeta potential measurements were made using a Zetasizer Nano (Malvern; Worcestershire, United Kingdom). In a typical experiment, silica particles, liposomes, or protocells were diluted 1:50 in a simulated body fluid (pH 7.4) or citric acid buffer (pH 5.0), both of which were adjusted to contain 150 mM NaCl, and transferred to 1-mL folded capillary cells (Malvern; Worcestershire, United Kingdom) for analysis.

### Synthesis, Loading, and Surface Functionalization of Protocells

### Liposome Fusion to Nanoporous Silica Particles

The procedure used to synthesize protocells has been described by Liu, et al.²⁵⁻²⁷ and will be mentioned only briefly. Lipids were ordered from Avanti Polar Lipids pre-dissolved in chloroform and stored at −20° C. Immediately prior to protocell synthesis, 2.5 mg of lipid was dried under a stream of nitrogen and placed in a vacuum oven (Model 1450M, VWR International, West Chester, Pa.) overnight to remove residual solvent. Lipids were re-hydrated in 0.5×D-PBS at a concentration of 2.5 mg/mL and were passed through a 100-nm filter at least 10 times using a Mini-Extruder set (Avanti Polar Lipids, Inc.; Alabaster, AL). DPPC and DSPC were dissolved in 0.5×D-PBS pre-warmed to their respective transition temperatures (41 ° C. and 55° C.) and maintained at 60° C. during the extrusion process. Resulting liposomes (˜120-nm in diameter) were stored at 4° C. for no more than one week. Nanoporous silica cores were dissolved in 0.5×D-PBS (25 mg/mL) and exposed to an excess of liposomes (1:2-1:4 volumetric ratio of lipid:silica) for 30-90 minutes at room temperature. Protocells were stored in the presence of excess lipid for up to 3 months at 4° C. To remove excess lipid, protocells were centrifuged at 10,000 rpm for 5 minutes, washed twice, and re-suspended in 0.5×D-PBS.

### Optimization of the Supported Lipid Bilayer Composition

The composition of the SLB was optimized to minimize non-specific binding and toxicity to control cells. The protocells used in all surface binding, internalization, and delivery experiments had SLBs composed of DOPC (or DPPC) with 5 wt % DOPE (or DPPE), 30 wt % cholesterol, and 5 wt % 18:1 (or 16:0) PEG-2000 PE. If necessary, fluorescent lipids (18:1-12:0 NBD-PC, 16:0-12:0 NBD-PC, or Texas Red® DHPE) were incorporated into the SLB at 1-5 wt %. Lipids were lyophilized together prior to rehydration and extrusion; for example 75 μL of DOPC (25 mg/mL), 5 μL of DOPE (25 mg/mL), 10 μL of cholesterol (75 mg/mL), 5 μL of 18:1 PEG-2000 PE (25 mg/mL), and 5 μL of 18:1-12:0 NBD-PC (5 mg/mL) were combined and dried to form liposomes composed of DOPC with 5 wt % DOPE, 30 wt % cholesterol, 5 wt % PEG-2000, and 1 wt % NBD-PC.

### Modification of the Supported Lipid Bilayer with Various Types of Targeting Ligands

The specific affinity of protocells for HCC was optimized by conjugating various types of targeting ligands in various densities to the SLB. The SP94 and HSWYG peptides (synthesized with C-terminal cysteine residues) were conjugated to primary amines present in the head groups of PE via the heterobifunctional crosslinker, NHS-(PEG)ₙ-maleimide, which is reactive toward sulfhydryl and amine moieties and possesses a PEG spacer arm, the length of which can be altered to optimize specific affinity. SM(PEG)₂₄ was used in most studies (spacer arm=9.52 nm). Amine moieties present in transferrin, anti-EGFR, and CHALV-1 were converted to free sulfhydryls using the Sulfhydryl Addition Kit (per manufacturer's instructions). Functionalized transferrin and antibodies were conjugated to PE in the SLB using SM(PEG)₂₄. Ligand density was controlled by both reaction stoichiometry and incubation time. For example, protocells were incubated with a 10-fold molar excess of SP94 for 2 hours at room temperature to attain a peptide density of 0.015 wt % (˜6 peptides/protocell), whereas protocells were incubated with a 5000-fold molar excess of SP94 overnight at 4° C. to attain a peptide density of 5.00 wt % (˜2048 peptides/protocell). Average ligand density was determined by Tricine-SDS-PAGE (SP94 and HSWYG peptides) or Laemmli-SDS-PAGE (transferrin, anti-EGFR, and CHALV-1)²⁸. Briefly, protocells were modified with various ligand densities using LC-SPDP (spacer arm=1.57 nm), a heterobifunctional crosslinker that reacts with primary amine and sulfhydryl moieties and is cleavable via reduction. Protocells were exposed to 10 mM dithiothreitol (DTT) for 30 minutes and centrifuged at 10,000 rpm for 5 minutes; the resulting supernatant contained free ligands, the concentration of which was determined via SDS-PAGE by comparing the band intensity of each sample to a standard curve using Image J Image Processing and Analysis software (National Institutes of Health; Bethesda, MD). 20% gels (with 6% bis-acrylamide and 6 M urea) were used to analyze the SP94 and HSWYG peptide densities. 10% gels were employed to analyze antibody (anti-EGFR and CHALV-1) densities, while 15% gels were used to analyze the density of transferrin.

### Preparation of Fluorescently-Labeled Nanoporous Cores

Nanoporous cores were fluorescently-labeled by adding 100 μL of particles (25 mg/mL) to 900 μL of 20% APTES in 0.5×D-PBS; the particles were incubated in APTES overnight at room temperature, centrifuged (10,000 rpm, 5 minutes) to remove unreacted APTES, and re-suspended in 1 mL of 0.5×D-PBS. An amine-reactive fluorophore (e.g. Alexa Fluor® 647 carboxylic acid, succinimidyl ester; 1 mg/mL in DMSO) was added (5 μL of dye per mL of particles), and the particles were kept at room temperature for 2 hours prior to being centrifuged to remove unreacted dye. Fluorescently-labeled particles were stored in 0.5×D-PBS at 4° C.

### Loading of Unimodal Cores and Liposomes with Chemotherapeutic Drugs

Prior to liposome fusion, unimodal nanoporous cores modified to contain 15 wt % AEPTMS (25 mg/mL) were soaked in doxorubicin (5 mM) or a mixture of doxorubicin, cisplatin, and 5-fluorouracil (5 mM of each drug) for 1 hour at room temperature. Excess drug was removed via centrifugation of the particles at 10,000 rpm for 5 minutes. 120-nm liposomes were loaded with DOX using an ammonium phosphate gradient-based method that has been described previously²⁹. Briefly, lipid films were re-hydrated with 300 mM (NH₄)₂HPO₄, and the liposome solution was extruded through a 100-nm membrane at least 10 times. Liposomes were equilibrated with an isotonic buffer solution (140 mM NaCl, 10 mM HEPES, pH 7.4) via dialysis (Float-A-Lyzer G2 dialysis units, 3.5-5 kDa MWCO; Spectrum Laboratories, Inc.; Rancho Dominguez, Calif.) and incubated with doxorubicin HCl (1:3 drug:lipid molar ratio) overnight at 4° C. Excess DOX was removed via size-exclusion chromatography on a 0.7 cm×10 cm Sepharose® CL-4B column. Liposomes were loaded with 5-FU or cisplatin as described previously^{30,31}.

### Loading of Multimodal Cores with the Multicomponent Mixture, siRNA, and Diphtheria Toxin A-Chain

Multimodal nanoporous cores modified to contain 20 wt % AEPTMS (25 mg/mL) were soaked in a solution of calcein (5 mM), Alexa Fluor® 647-labeled dsDNA oligonucleotides (100 μM), RFP (100 μM), and CdSe/ZnS quantum dots (10 μM) for 4 hours; the concentration of each cargo was varied in order to attain the optimal fluorescence intensity for hyperspectral imaging. Calcein was modified with the NLS (synthesized with a C-terminal cysteine residue) by dissolving 1 mg each of calcein and the NLS in 850 μL of 1× D-PBS; 100 μL of EDC (10 mg/mL in deionized water) and 50 pL of BMPH (10 mg/mL in DMSO) were added, and the mixture was incubated for 2 hours at room temperature. Excess calcein was removed via dialysis (Slide-A-Lyzer mini dialysis units, 2 kDa MWCO; Thermo Fisher Scientific LSR; Rockford, Ill.). The dsDNA oligonucleotide was labeled using the Ulysis™ Alexa Fluor® 647 Nucleic Acid Labeling Kit (per manufacturer's instructions) and modified with the NLS by combining 50 μL of dsDNA (2 mM in deionized water) with 50 μL of the NLS (1 mM in DMSO) and 10 μL of SMCC (10 mg/mL in DMSO); the mixture was incubated at room temperature for 2 hours, and excess NLS was removed via dialysis (Slide-A-Lyzer mini dialysis units, 7 kDa MWCO; Thermo Fisher Scientific LSR; Rockford, Ill.). Multimodal nanoporous cores modified with 20 wt % AEPTMS (25 mg/mL) were soaked in siRNA (100 μM) or diphtheria toxin A-chain (100 μM) for 2 hours at 4° C. Unencapsulated cargo was removed via centrifugation at 10,000 rpm for 5 minutes, and liposomes were immediately fused to cargo-loaded cores.

### Packaging of the CB1 Plasmid with Histone Proteins

The process used to supercoil the CB1 plasmid (pCB1) employed a highly saturated salt solution, package supercoiled pCB1 with histones H1, H2A, H2B, H3, and H4, and modifying the resulting pCB1-histone complex with a nuclear localization sequence (NLS) that promotes translocation through nuclear pores by conjugation to histone protein.

### Synthesis of MC4O-Targeted Mesoporous Silica Nanoparticle-Supported Lipid Bilayers (Protocells) Loaded with Histone-Packaged pCB1

Histone-packaged pCB1 is loaded into the mesoporous silica nanoparticles that form the core of the protocell by simply soaking the particles in a solution of the pCB1-histone complex. PEGylated liposomes are then fused to DNA-loaded cores to form a supported lipid bilayer (SLB) that is further modified with a targeting peptide (MC40) that binds to HCC and a endosomolytic peptide (HSWYG) that promotes endosomal escape of internalized protocells. A sulfhydryl-to-amine crosslinker (spacer arm=9.5 nm) was used to conjugate peptides, modified with a C-terminal cysteine residue, to DOPE moieties in the SLB. SEM of the mesoporous silica nanoparticles demonstrates that the 15-25 nm pores are surface-accessible.

### Mesoporous Silica Nanoparticles have a High Capacity for Histone-Packaged pCB1, and the Resulting Protocells Release Encapsulated DNA Only Under Conditions that Mimic the Endosomal Environment

The concentration of pCB1 or histone-packed pCB1 (complex') that can be encapsulated within unmodified mesoporous silica nanoparticles (ζ=−38.5 mV) or mesoporous silica nanoparticles modified with APTES, an amine-containing silane (ζ=+11.5 mV), was determined. For example, the percentage of Hep3B that become positive for ZsGreen, a green fluorescent protein encoded by pCB1, when 1×10⁶ cells/mL are incubated with 1×10⁹ MC40-targeted, pCB1 loaded protocells for 24 hours at 37° C. was determined. The x-axis specifies whether the protocell core was modified with APTES and whether pCB1 was pre-packaged with histones. pCB1 packaged with a mixture of DOTAP and DOPE (1:1 tier/w) was included as a control. A time-dependent release of histone-packaged pCB1 from unmodified mesoporous silica nanoparticles and corresponding protocells was shown upon exposure to a simulated body fluid or a pH 5 buffer. The protocell SLB was composed of DOPC with 5 wt % DOPE, 30 wt % cholesterol, and 10 wt % PEG-2000 or was modified with 0.015 wt % MC40 and 0.500 wt % H5WYG.

### The Process by which MC40-Targeted Protocells Deliver Histone-Packaged pCB1 to HCC

MC40-targeted protocells bind to Hep3B cells with high affinity due to the recruitment of targeting peptides to Met, which is over-expressed by a variety of HCC lines. The fluid DOPC SLB promotes peptide mobility and, therefore, enables protocells modified with a low MC40 density to retain a high specific affinity for Hep3B. MC40-targeted protocells become internalized by Hep3B via receptor-mediated endocytosis. Endosomal conditions destabilize the SLB [insert Nature Materials ref] and cause protonation of the H5WYG endosomolytic peptide, both of which enable histone-packaged pCB1 to become dispersed in the cytosol of Hep3B cells. pCB1-histone complexes, when modified with a nuclear localization sequence (NLS), become concentrated in the nuclei of Hep3B cells within ˜24 hours, which enables efficient transfection of both dividing and non-dividing cancer cells.

### MC40-Targeted Protocells Bind to HCC with High Affinity and are Internalized by Hep3B but not by Normal Hepatocytes

The apparent dissociation constants (K_{d}) for MC40-targeted protocells when exposed to Hep3B or hepatocytes was determined; K_{d} values are inversely related to specific affinity and were determined from saturation binding curves. Error bars represent 95% confidence intervals (1.96σ) for n=5. Confocal fluorescence microscopy images of Hep3B (B) and hepatocytes (C) that were exposed to a 1000-fold excess MC40-targeted protocells for 1 hour at 37° C. were observed. Met was stained with an Alexa Fluor® 488-labeled monoclonal antibody (green), the protocell core was labeled with Alexa Fluor® 594 (red), and cell nuclei were stained with Hoechst 33342 (blue). Scale bars=20 μm. Protocell SLBs were composed of DOPC with 5 wt % DOPE, 30 wt % cholesterol, and 10 wt % PEG-2000 (18:1) and were modified with either 0.015 wt % (A-C) or 0.500 wt % (A) of the MC40 targeting peptide.

### MC40-Targeted, pCB1-Loaded Protocells Induce Apoptosis of HCC at Picomolar Concentrations but have a Minimal Impact on the Viability of Normal Hepatocytes

The dose and dependent decreases in expression of cyclin B1 mRNA and cyclin B1 protein upon continual exposure of Hep3B to MC40-targeted, pCB1-loaded protocells at 37° C. were determined. Cells were exposed to various pCB1 concentrations for 48 hours in and to 5 pM of pCB1 for various periods of time. Expression of cyclin B1 protein in hepatocytes and ZsGreen in Hep3B are included as controls. Real-time PCR and immunofluorescence were employed to determine cyclin B1 mRNA and protein concentrations, respectively. The percentage of Hep3B that become arrested in G₂/M phase after continual exposure to MC40-targeted, pCB1-loaded protocells ([pCB1]=5 pM) for various periods of time at 37° C. was determined. The percentage of hepatocytes in G₂/M phase and Hep3B in S phase are included for comparison. Cells were stained with Hoechst 33342 prior to cell cycle analysis. The percentage of Hep3B that become apoptotic upon continual exposure to MC40-targeted, pCB1-loaded protocells ([pCB1]=5 pM) for various periods of time at 37° C. was also determined. The percentage of hepatocytes positive for markers of apoptosis was included as a control. Cells positive for Alexa Fluor® 647-labeled annexin V were considered to be in the early stages of apoptosis, while cells positive for both annexin V and propidium iodide were considered to be in the late stages of apoptosis. The total number of apoptotic cells was determined by adding the numbers of single- and double-positive cells. In all experiments, protocell SLBs were composed of DOPC with 5 wt % DOPE, 30 wt % cholesterol, and 10 wt % PEG-2000 (18:1) and were modified with 0.015 wt % MC40 and 0.500 wt % HSWYG.

### MC40-Targeted, pCB1 Loaded Protocells Induce Selective Apoptosis of HCC 2500-Fold More Effectively than Corresponding Lipoplexes

The zeta potential values for DOPC protocells, DOPC protocells modified with 10 wt % PEG-2000 (18:1), lipoplexes composed of pCB1 and a mixture of DOTAP and DOPE (1:1 w/w), and DOTAP/DOPE lipoplexes modified with 10 wt % PEG-2000, was determined All zeta potential measurements were conducted in 0.5×PBS (pH 7.4). The percentage of Hep3B and hepatocytes that become apoptotic upon continual exposure to 5 pM of pCB1, delivered via MC40-targeted protocells or lipoplexes, for 48 hours at 37° C. was determined, as well as the number of MC40-targeted, pCB1-loaded protocells or lipoplexes necessary to induce apoptosis in 90% of 1×10⁶ Hep3B cells within 48 hours at 37° C. Cells were stained with Alexa Fluor® 647-labeled annexin V and propidium iodide; single- and double-positive cells were considered to be apoptotic. Protocell SLBs were composed of DOPC with 5 wt % DOPE, 30 wt % cholesterol, and 10 wt % PEG-2000 (when indicated) and were modified with 0.015 wt % MC40 and 0.500 wt % HSWYG. DOTAP/DOPE lipoplexes were modified with 10 wt % PEG-2000 (when indicated), 0.015 wt % MC40, and 0.500 wt % HSWYG. pCB1 was modified with the NLS in all experiments.

### MC40-Targeted Protocells Selectively Deliver High Concentrations of Taxol, BcI-2-Specific siRNA, and pCB1 to HCC Without Affecting the Viability of Hepatocytes

The concentrations of taxol, siRNA that silence expression of BcI-2, and the CB1 plasmid that can be encapsulated within 10¹² protocells, liposomes, or lipoplexes were determined. Taxol and pCB1 concentrations change when both are loaded within protocells. Taxol, siRNA, and pCB1 concentrations change when all three are loaded within protocells or when siRNA and pCB1 are loaded within lipoplexes. Confocal fluorescence microscopy image shows the intracellular distributions of Oregon Green® 488-labeled taxol (green), Alexa Fluor® 594-labeled siRNA (red), and Cy5-labeled pDNA (white) upon delivery to Hep3B via MC40-targeted protocells. Cells were incubated with a 1000-fold excess of MC40-targeted protocells for 24 hours at 37° C. prior to being fixed and stained with Hoechst 33342 (blue). The fractions of Hep3B, SNU-398, and hepatocyte cells that become arrested in G₂/M phase upon exposure to 10 nM of taxol and/or 5 pM of pCB1 for 48 hours at 37° C. were determined. Fractions were normalized against the percentage of logarithmically-growing cells in G₂/M. The percentage of Hep3B, SNU-398, and hepatocyte cells that become positive for Alexa Fluor® 647-labeled annexin V and propidium iodide (PI) upon exposure to 10 nM of taxol, 250 pM of BcI-2-specific siRNA, and/or 5 pM of pCB1 for 48 hours at 37° C. was also determined. ‘pCB1’ refers to pCB1 that was packaged and delivered non-specifically to cells using a mixture of DOTAP and DOPE (1:1 w/w). In all experiments, protocell SLBs were composed of DOPC with 5 wt % DOPE, 30 wt % cholesterol, and 10 wt % PEG-2000 (18:1) and were modified with 0.015 wt % MC40 and 0.500 wt % HSWYG. Liposomes were composed of DSPC with 5 wt % DMPE, 30 wt % cholesterol, and 10 wt % PEG-2000 (16:0) and were modified with 0.015 wt % MC40 and 0.500 wt % HSWYG. Lipoplexes were composed of a DOTAP:DOPE (1:1 w/w) mixture and were modified with 10 wt % PEG-2000, 0.015 wt % MC40, and 0.500 wt % HSWYG. pCB1 was modified with the NLS in all experiments.

### Vector Map for the CB1 Plasmid

The CB1 plasmid (pCB1) was constructed from the RNAi-Ready pSIREN-RetroQ-ZsGreen vector (Clontech Laboratories, Inc.; Mountain View, Calif.) and the pNEB193 vector (New England BioLabs, Inc.; Ipswich, Mass.). pCB1 encodes a cyclin B1-specific small hairpin RNA (shRNA) [Yuan, et al., Oncogene (2006) 25, 1753-1762] and a Zoanthus sp. green fluorescent protein (ZsGreen). Constitutive shRNA expression is driven by the RNA Pol III-dependent human U6 promoter (P_{U6}), while constitutive ZsGreen expression is driven by the immediate early promoter of cytomegalovirus (P_{CMV IE}). The ori and Amp^{R} elements enable propagation of the plasmid in E. coli. The DNA sequences that encode the sense and antisense strands of the cyclin B1-specific shRNA are underlined and are flanked by the restriction enzyme sites (BamHI in red and EcoRl in blue) that were employed to introduce the dsDNA oligonucleotide into the pSIREN vector.

### Characterization of Histone-Packaged pCB1

Electromobility shift assays were conducted for pCB1 exposed to increasing concentrations of histones (H1, H2A, H2B, H3, and H4 in a 1:2:2:2:2 molar ratio). The pCB1:histone molar ratio is given for lanes 3-6. Lane 1 contains a DNA ladder, and lane 2 contains pCB1 with no added histones.

### Nitrogen Sorption Analysis of Unloaded and pCB1 Loaded Mesoporous Silica Nanoparticles

Nitrogen sorption isotherms were determined for mesoporous silica nanoparticles before and after loading with histone-packaged pCB1. The Brunauer-Emmett-Teller (BET) surface area of mesoporous silica nanoparticles, before and after loading with histone-packaged pCB1, was also determined.

### Small-Angle Neutron Scattering (SANS) Data for DOPC Protocells

The data fit for SANS data for DOPC protcells was obtained using a model for polydisperse porous silica spheres with a conformal shell of constant thickness and shows the presence of a 36-Å bilayer at the surface of the silica particles that spans pore openings. Simulated SANS data for bilayer thicknesses of 0, 20, and 60 Å are included for comparison. The measured bilayer thickness of 36 Å is consistent with other neutron studies (33-38 Å) [see, Ferrari, M. Cancer nanotechnology: Opportunities and challenges. Nature Reviews Cancers, 161-171 (2005)] performed on planar supported lipid bilayers and, under these contrast conditions, primarily represents scattering from the hydrogen-rich hydrocarbon core of the lipid bilayer. Experimental data also demonstrates the presence of 299.2-A pores, determined by dividing 0.0315 Å⁻¹ (i.e. the q.-value for the peak in the experimental data, which is caused by scattering from pores) into 2ρ. SANS data were obtained on the LQD beam line at LANSCE (Los

Alamos National Laboratories) using a 5% (v/v) protocell suspension in 100% D₂O PBS buffer. Data were fit using the NCNR SANS data analysis package (NIST).

### Protocells Protect Encapsulated DNA from Nuclease Degradation

Agarose gel electrophoresis of DNase I-treated pCB1, histone-packaged pCB1, pCB1 packaged with a 1:1 (w/w) mixture of DOTAP and DOPE, pCB1 loaded in protocells with cationic cores, and histone-packaged pCB1 loaded in protocells with anionic cores was conducted. Naked pCB1, pCB1 released from histones, pCB1 released from DOTAP/DOPE lipoplexes), pCB1 released from protocells with cationic cores, and histone-packaged pCB1 released from protocells with anionic cores are included for comparison. Samples were incubated with DNase 1 (1 unit per 50 ng of DNA) for 30 minutes at room temperature, and pCB1 release was stimulated using 1% SDS.

The Zeta potential (ζ)values for mesoporous silica nanoparticles (‘unmodified cores’), mesoporous silica nanoparticles that were soaked in 20% (viv) APTES for 12 hours at room temperature (‘APTES-modified cores’), the CB1 plasmid (‘pCB1’), histone-packaged pCB1 (‘pCB1-histone complex’), and pCB1 packaged with a 1:1 (w/w) mixture of DOTAP and DOPE (‘DOTAP/DOPE Lipoplexes’) was determined. Zeta potential measurements were conducted in 0.5×PBS (pH 7.4).

### Representative Forward Scatter-Side Scatter (FSC-SSC) Plots and FLA Histograms Used to Determine the Percentage of Cells Positive for ZsGreen Expression Identification of the MC40 Targeting Peptide

A process was used to select the MC40 targeting peptide from a Ph.D.™ 7 phage display library (New England BioLabs, Inc.; Ipswich, Mass.). 1×10¹¹ pfu/mL were incubated with 100 nM of recombinant human Met (rhMet), fused to the Fc domain of human IgG, for 1 hour at room temperature. Protein A or protein G-coated magnetic particles were used to affinity capture Met-phage complexes and were subsequently washed 10 times with TBS (50 mM Tris-HCl with 150 mM NaCl, pH 7.4) to remove unbound phage. Bound phage clones were eluted with a low-pH buffer (0.2 M glycine with 1 mg/mL BSA, pH 2.2), and elutants were amplified via infection of the host bacterium (E. coli ER2738). Pursuant to the schematic, five rounds of affinity selection were performed using increasingly stringent conditions: the Met concentration was decreased from 100 nM to 50 nM to 10 nM, the incubation time was reduced from 1 hour to 30 minutes to 15 minutes, and the concentration of Tween-20 added to the wash buffer was increased from 0% (v/v) to 0.1% to 0.5%. Peptides specific for protein A and protein G were avoided by alternating rounds of selection between protein A-coated magnetic particles and protein G-coated magnetic particles. After five rounds of selection, DNA was recovered from 40 individual clones and sequenced using the -96 gill primer provided with the Ph.D™-7 kit. The sequenes which have the greatest binding activity against the MET receptor are presented as follows:

SEQ ID NO: 1
ASVHFPP (Ala-Ser-Val-His-Phe-Pro-Pro)
SEQ ID NO: 2
TATFWFQ (Thr-Ala-Thr-Phe-Trp-Phe-Gln)
SEQ ID NO: 3
TSPVALL (Thr-Ser-Pro-Val-Ala-Leu-Leu)
SEQ ID NO: 4
IPLKVHP (Ile-Pro-Leu-Lys-Val-His-Pro)
SEQ ID NO: 5
WPRLTNM (Trp-Pro-Arg-Leu-Thr-Asn-Met)

### Characterization of the MC40 Targeting Peptide

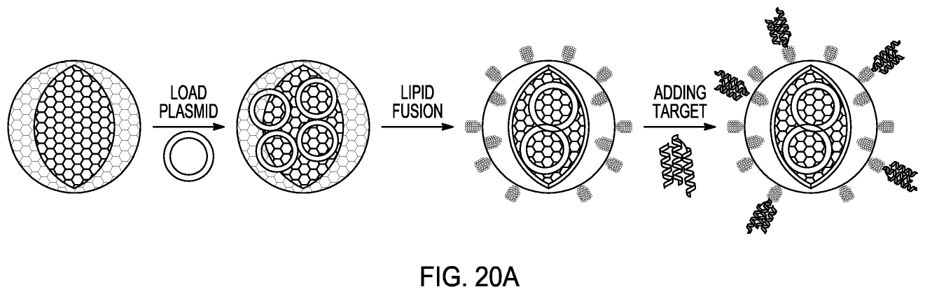

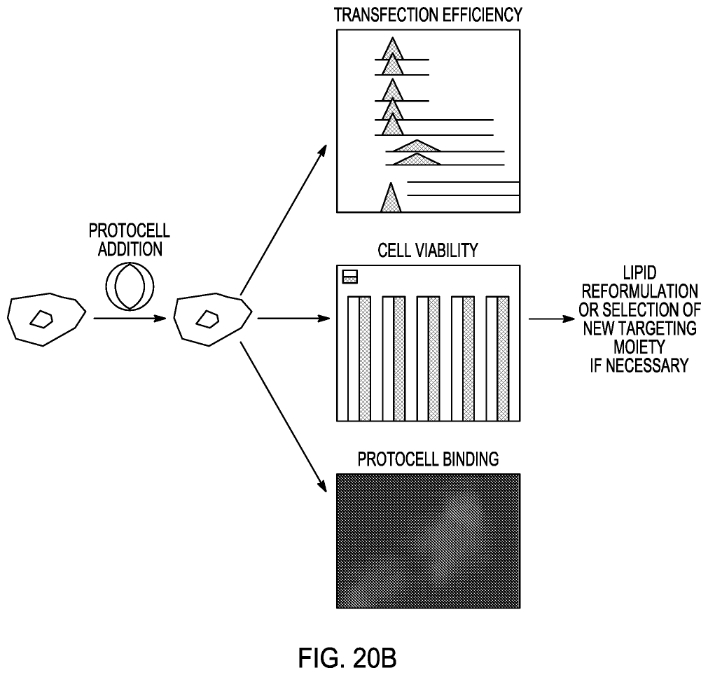

The predominant sequence, ASVHFPP, is similar to the emboldened portion of a previously-identified Met-specific 12-mer, YLFSVHWPPLKA SEQ ID NO: 18). Phage clones displaying the target-unrelated HAIYPRH peptide (about 10%) (SEQ ID NO: 19) were omitted from the sequence alignment. The degree to which affinity-selected phage clones bound to rhMet was determined via enzyme-linked immunosorbent assay (ELISA) was detected. The ELISA scheme, depicted in (B), is described in the Materials and Methods section. ELISA results are shown in (C). FIG. 20(D) shows the sequence alignment after peptides that do not bind to Met were removed. The consensus sequence depicted in FIG. 20 was determined from this alignment. Flow cytometry scatter plots for Hep3B (E) and hepatocytes (F) exposed to either (1) an Alexa Fluor® 488-labeled monoclonal antibody against Met AND an irrelevant phage clone (TPDWLFP) AND an Alexa Fluor® 546-labeled monoclonal antibody against M13 phage or (2) an Alexa Fluor® 488-labeled monoclonal antibody against Met AND the MC40 clone AND an Alexa Fluor® 546-labeled monoclonal antibody against M13 phage, were prepared. Untreated cells were used to set voltage parameters for the FL-1 (Alexa Fluor® 488 fluorescence) and FL-2 (Alexa Fluor® 546 fluorescence) channels.

### Sample Binding Curves for MC40-Targeted Protocells Exposed to Hep3B

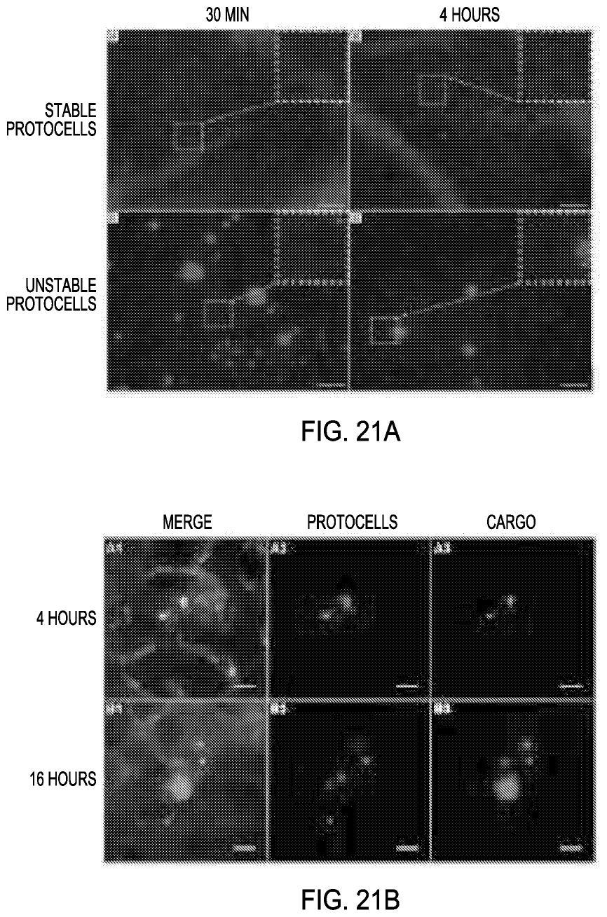

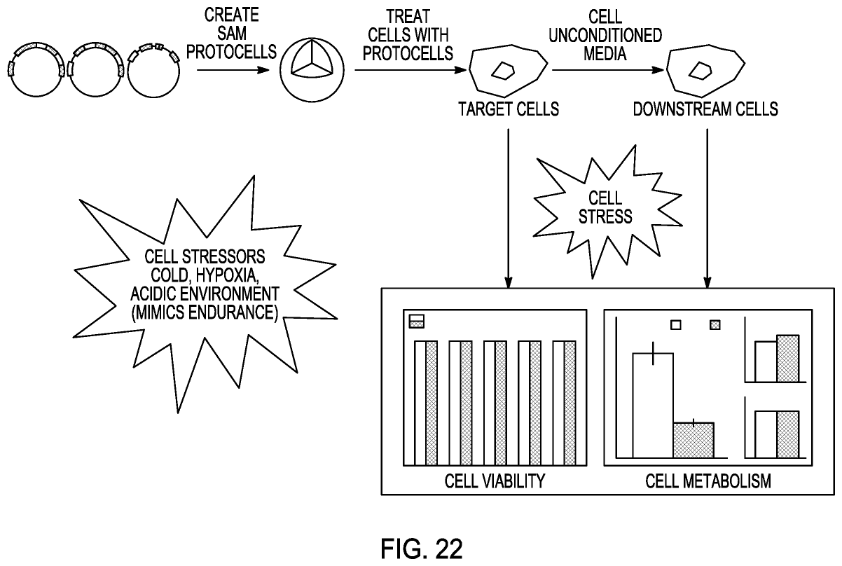

To determine the dissociation constants, 1×10⁶ Hep3B or hepatocytes were pre-treated with cytochalasin D to inhibit endocytosis and incubated with various concentrations of Alexa Fluor® 647-labeled, MC40-targeted protocells for 1 hour at 37′C. Flow cytometry was used to determine mean fluorescence intensities for the resulting cell populations, which were plotted against protocell concentrations to obtain total binding curves. Non-specific binding was determined by incubating cells with Alexa Fluor® 647-labeled, MC40-targeted protocells in the presence of a saturating concentration of unlabeled hepatocyte growth factor. Specific binding curves were obtained by subtracting non-specific binding curves from total binding curves; K_{d} values were calculated from specific binding curves. In the experiments which are depicted in FIG. 21, protocell SLBs were composed of DOPC with 5 wt % DOPE, 30 wt % cholesterol, and 10 wt % PEG-2000 (18:1) and were modified with 0.015 wt % (about 6 peptides/particle) of the MC40 targeting peptide; the corresponding K_{d} value is 1050±142 pM.

### MC40-Targeted Protocells are Internalized Via Receptor-Mediated Endocytosis and, in the Absence of the H5WYG Peptide, are Directed to Lysosomes

The average number of MC40-targeted protocells internalized by each Hep3B or hepatocyte cell within one hour at 37° C. 1×10⁶ cells were incubated with various concentrations of protocells in the absence (−) or presence (+) of a saturating concentration (100 μg/mL) of human hepatocyte growth factor (HGF) was determined, and flow cytometry was used to determine the average number of particles associated with each cell, as described by Ashley, et al. Nature Materials, 2011, May; 10(5):389-97. Protocells were labeled with NBD and pHrodo™ to distinguish surface-bound particles from those internalized into acidic intracellular compartments (respectively). Error bars represent 95% confidence intervals (1.96σ) for n=3. Pearson's correlation coefficients (r-values) between protocells and: (1) Rab5, (2) Rab7, (3) Lysosomal-Associated Membrane Protein 1 (LAMP-1), or (4) Rab11a. Hep3B cells were incubated with a 1000-fold excess of Alexa Fluor® 594-labeled protocells for 1 hour at 37° C. before being fixed, permeabilized, and incubated with Alexa Fluor® 488-labeled antibodies against Rab5, Rab7, LAMP-1, or Rab11a. SlideBook software was used to determine r-values, which are expressed as the mean value ±the standard deviation for n=3×50 cells. Differential Interference Contrast (DIC) images were employed to define the boundaries of Hep3B cells so that pixels outside of the cell boundaries could be disregarded when calculating r-values. Protocell SLBs were composed of DOPC with 5 wt % DOPE, 30 wt % cholesterol, and 10 wt % PEG-2000 (18:1) and were modified with 0.015 wt % MC40 and 0.500 wt % H5WYG.

### Histone-Packaged pCB1, when Modified with a NLS and Delivered via MC40-Targeted Protocells, becomes Concentrated in the Nuclei of HCC Cells in a Time-Dependent Manner

Confocal fluorescence microscopy images of Hep3B cells exposed to a 1000-fold excess of MC40-targeted, pCB1-loaded protocells for 15 minutes (A), 12 hours (B), or 24 hours (C) at 37° C. were reviewed. For (B) ndosomal escape of protocells and cytosolic dispersion of pCB1 was evident after ˜2 hours; ZsGreen expression was not detectable until 12-16 hours, however. At 24 hours, Cy5-labeled pCB1 remained distributed throughout the cells; cytosolic staining is not visible in (C), however, since the gain of the Cy5 channel was reduced to avoid saturation of pixels localized within the nuclei. Silica cores were labeled with Alexa Fluor® 594 (red), pCB1 was labeled with Cy5 (white), and cell nuclei were counterstained with Hoechst 33342 (blue). Scale bars=20 μm. Pearson's correlation coefficients (r-values) versus time for Cy5-labeled pCB1 and Hoechst 33342-labeled Hep3B nuclei were determined. SlideBook software was used to determine r-values, which are expressed as the mean value±the standard deviation for n=3×50 cells. Differential Interference Contrast (DIC) images were employed to define the boundaries of Hep3B cells so that pixels outside of the cell boundaries could be disregarded when calculating r-values. Protocell SLBs were composed of DOPC with 5 wt % DOPE, 30 wt % cholesterol, and 10 wt % PEG-2000 (18:1) and were modified with 0.015 wt % MC40 and 0.500 wt % H5WYG.

### Histone-Packaged pCB1, when Modified with a NLS and Delivered via MC40-Targeted Protocells, Selectively Transfects Both Dividing and Non-Dividing HCC Cells with Nearly 100% Efficacy

Confocal fluorescence microscopy images of Hep3B cells exposed to a 1000-fold excess of MC40-targeted, pCB1-loaded protocells for 24 hours at 37° C., were obtained. Hep3B cells were dividing in (A) and ˜95% confluent in (C) and (E); pCB1 was pre-packaged with histones in all images, and the pCB1-histone complex was further modified with a NLS in (E). Silica cores were labeled with Alexa Fluor® 594 (red), pCB1 was labeled with Cy5 (white), and cell nuclei were counterstained with Hoechst 33342 (blue). Scale bars=20 μm. FIGS. 24(B), (D), and (F) show the percentage of 1×10⁶ Hep3B and hepatocytes that become positive for ZsGreen expression upon continual exposure to 1×10⁹ MC40-targeted, pCB1-loaded protocells (‘PC’) for 24 hours at 37° C. Cells were dividing in (B) and ˜95% confluent in (D) and (F); the x-axes indicate whether CB1 plasmids (‘pCB1’) and pCB1-histone complexes (‘complex’) were modified with the NLS. pCB1 alone, as well as pCB1 packaged with a 1:1 (w/w) mixture of DOTAP and DOPE were employed as controls. Cells were exposed to 20 mg/mL of wheat germ agglutinin (WGA) to block translocation of NLS-modified pCB1 through the nuclear pore complex. Error bars represent 95% confidence intervals (1.96σ) for n=3. Cell cycle histograms for cells employed in (A), (C), and (E), respectively, were prepared. The percentage of cells in G₀/G₁ phase is given for each histogram. In all experiments, protocell SLBs were composed of DOPC with 5 wt % DOPE, 30 wt % cholesterol, and 10 wt % PEG-2000 (18:1) and were modified with 0.015 wt % MC40 and 0.500 wt % H5WYG.

Confocal fluorescence microscopy images of Hep3B (A) and hepatocytes (B) that were exposed to MC40-targeted, pCB1-loaded protocells for either 1 hour or 72 hours at 37° C. were obtained; the pCB1 concentration was maintained at 5 pM in all experiments. The arrows in (B) indicate mitotic cells. Cyclin B1 was labeled with an Alexa Fluor® 594-labeled monoclonal antibody (red), and cell nuclei were stained with Hoechst 33342 (blue). Protocell SLBs were composed of DOPC with 5 wt % DOPE, 30 wt % cholesterol, and 10 wt % PEG-2000 (18:1) and were modified with 0.015 wt % MC40 and 0.500 wt % H5WYG. All scale bars=20 μm.

Confocal fluorescence microscopy images were obtained of Hep3B (A) and hepatocytes (B) that were exposed to MC40-targeted, pCB1 loaded protocells for either 1 hour or 72 hours at 37° C.; the pCB1 concentration was maintained at 5 pM in all experiments. Cells were stained with Alexa Fluor® 647-labeled annexin V (white) and propidium iodide (red) to assay for early and late apoptosis, respectively, and cell nuclei were counterstained with Hoechst 33342 (blue). Protocell SLBs were composed of DOPC with 5 wt % DOPE, 30 wt % cholesterol, and 10 wt % PEG-2000 (18:1) and were modified with 0.015 wt % MC40 and 0.500 wt % HSWYG.

### Protocells with a SLB Composed of Zwitterionic Lipids Induce Minimal Non-Specific Cytotoxicity

The percentage of 1×10⁶ Hep3B that become apoptotic upon continual exposure to 1×10⁹ APTES-modified mesoporous silica nanoparticles, DOPC protocells with APTES-modified cores, DOPC protocells loaded with a plasmid that encodes a scrambled shRNA sequence (‘scrambled pCB1’), or DOTAP/DOPE (1:1 w/w) lipoplexes loaded with scrambled pCB1 for 48 hours at 37° C. was determined. Protocells and lipoplexes were modified with 10 wt % PEG-2000, 0.015 wt % MC40, and 0.500 wt % H5WYG. Positively- and negatively-charged polystyrene nanoparticles (‘amine-PS’ and ‘Carboxyl-PS’, respectively) were employed as positive controls, while Hep3B exposed to 10 mM of the antioxidant, N-acetylcysteine (NAC), or to 1 pmol of free pCB1 were used as negative controls.

The following non-limiting examples are illustrative of the disclosure and its advantageous properties, and are not to be taken as limiting the disclosure or claims in any way. In the examples, as well as elsewhere in this application, all parts and percentages are by weight unless otherwise indicated.

### Example 2

An exemplary transcriptional mediator plasmid includes commercially available plasmids that encode for an engineered CRISPR Cas9. While the standard CRISPR Cas9 will cleave the DNA target, this engineered version only binds the targeted DNA sequence and a second engineered site on the CRISPR Cas9 mutant recruits transcriptional activators or repressors to the targeted gene. This results in either increased or decreased gene expression.

Example A. Nonspecific protocells used to deliver a green fluorescent protein (GFP) reporter plasmid. This plasmid was utilized for testing of various non-specific SLB formulations for plasmid delivery utilizing protocells. Formulations utilized were: 1) 100% DOTAP, 2) 55% DOTAP, 5% DOPE, 30% Cholesterol, 10% 1,2-dioleoyl-sn-glycero-3-phosphoethanolamine-N-[methoxy(polyethylene glycol)-2000] (DOPE-PEG 2000), 3) 55% DOTAP, 5% DOPE, 30% Cholesterol, 10% 1,2-distearoyl-sn-glycero-3-phosphoethanolamne-N-[methoxy(polyethylene glycol)-2000] (DSPE-PEG 2000). Protocells were loaded with GFP plasmid DNA and then used to treat a prostate cancer cell line. After 12 hours, protocells that had not been internalized were removed. The cells were allowed to grow for 72 hours to allow expression of the GFP protein. Cells were collected and the percentage of cells expressing GFP was determined using flow cytometry. All three formulations showed a significant increase in GFP expression over control.

Example B. Nonspecific protocells, utilizing 100% DOTAP SLBs were loaded with a Transcriptional activator plasmid set, the Synergistic Activation Mediator (SAM) system. Essentially this is a 3 plasmid construct that, once transcribed, will promote the synthesis and assembly of the SAM complex components. These include a deactivated Cas9-VP64 protein fusion structure that confers site specificity but doesn't actually cleave DNA, and sgRNA that incorporates two RNA aptamers at the tetraloop and stem-loop 2, and a third plasmid which expresses the activation helper protein. Once these components assemble, they target a selected region of DNA depending on the intended function of the system. The SAM complex will bind upstream of the transcriptional start site of the gene of interest, in our case, we chose to target tumor protein 53, or p53, and it will force expression of that gene. This SAM system was loaded into both the protocell (Protocell+SAM) and into the recommended control transaction agent (Control SAM Transfection). The expression of p53 was then compared to the protocell without any SAM. Microscopy was utilized to visualize the presence of protocells (red) associated with the cells (nuclei stained blue) and the expression of p53 protein (green) compared to untreated cells (FIG. 2). Microscopy showed a significant increase in p53 expression relative to background.

Quantififcation of the expression of p53 by flow cytometry (FIG. 3) showed an increase in p53 with delivery by protocell compared to the recommended transfection agent (SAM control).

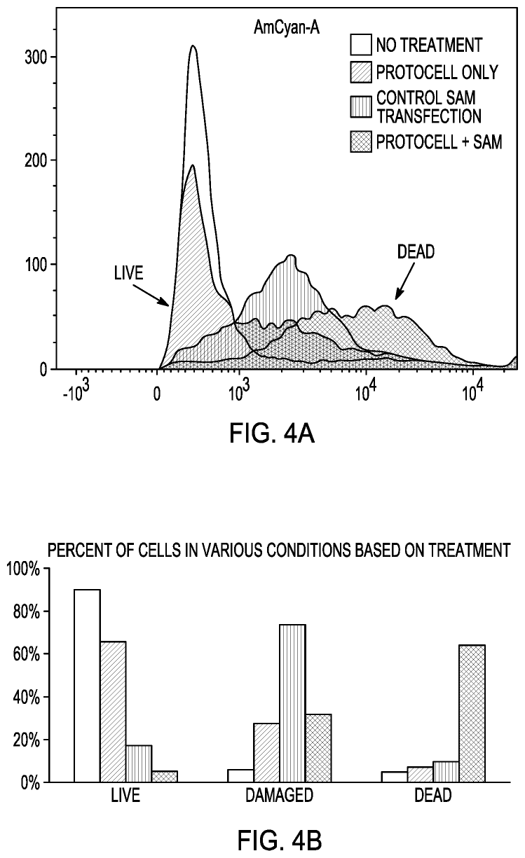

Example C. Delivery of p53 transcriptional activator (SAM) by protocell results in expression of an aberrant protein in prostate cancer cell line PC-3 which results in cell death. PC-3 cells were treated with protocells alone, protocells loaded with SAM, the commercial SAM transfection agent or left untreated. After 24 hours, cells were assessed for viability using Life Technology Live/Dead® Fixable Dead Cell Stain Kit in Aqua fluorescent one-channel assay utilizing the standard protocol by flow cytometry (FIG. 4). Treatment with protocells alone did not result in any cell death. However, treatment with the SAM loaded protocells resulted in a significant portion of the cells initiating cell death, even when compared to the recommended treatment agent.

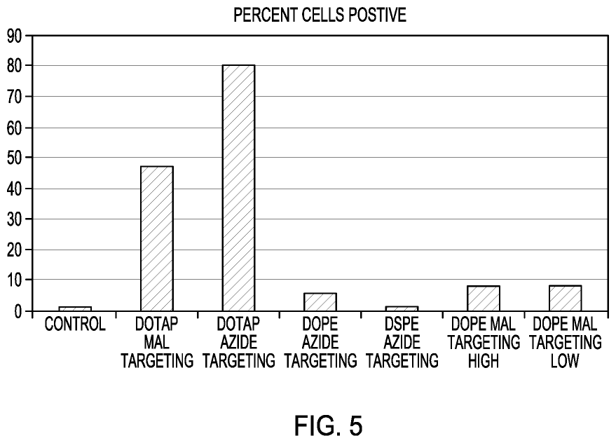

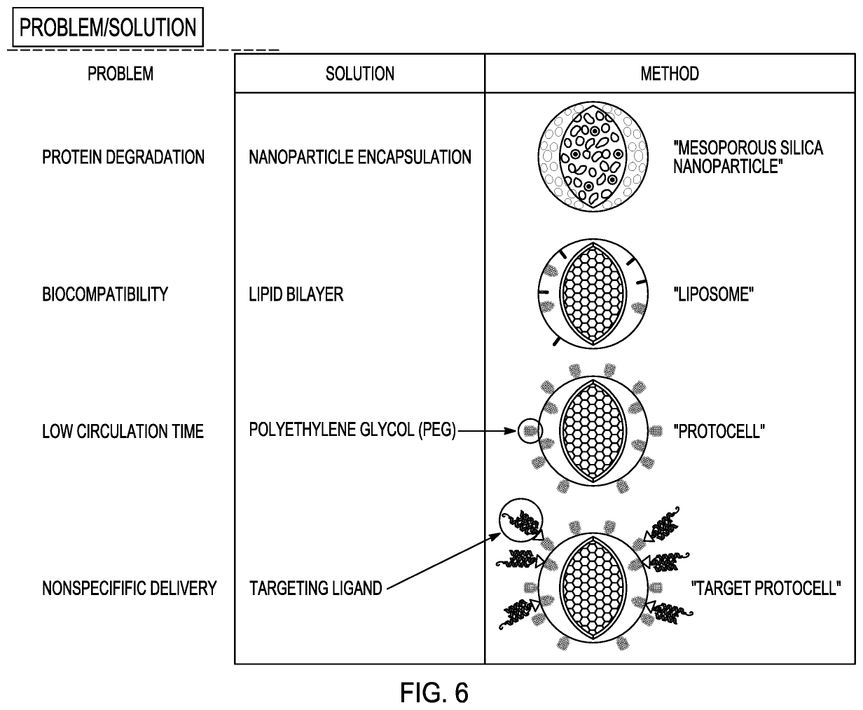

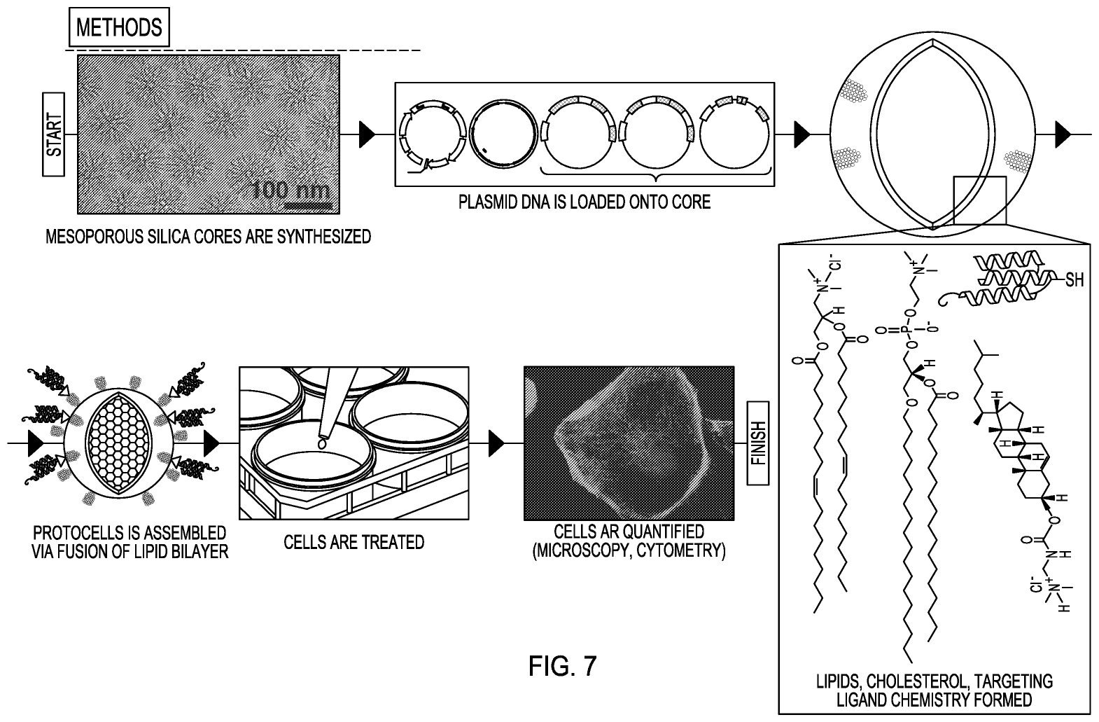

Example D. Affibody targeted protocells used to deliver a green fluorescent protein (GFP) reporter plasmid. This plasmid was utilized for testing of various targeted SLB formulations for plasmid delivery utilizing protocells. Cells were exposed to protocells utilizing affibody targeting to specifically deliver GFP plasmid and the expression of the GFP protein was assessed by flow cytometry (FIG. 5). All formulations which utilized DOPE demonstrated significant delivery and expression of GFP protein. The use of DOTAP to create a hybrid specific/non-specific protocell resulted in the highest level of GFP expression. By comparison, DSPE based targeted delivery did not result in any expression of GFP protein.

### Example 3

Human adaptation to new environments occurs slowly through biological evolution or more rapidly through acclimatization, wherein environmental exposure leads progressively to adaptive alterations in gene expression; these alterations may allow an individual to function under extreme conditions to which the individual has never been exposed. However, these alterations require weeks to months to occur, which greatly limits the ability of a human to rapidly adapt to extreme environments or increase athletic endurance. Rapid ‘on-demand’ and reversible adaptation can be induced by utilizing CRIPSR-enabled SAM technologies to alter gene expression, allowing humans to pre-adapt to extreme conditions or increase endurance for weeks or months without the need for slow, progressive increase in environmental exposure or high altitude training.

Protocell delivery of CRISPR Synergistic Activation Mediator (SAM) system. The SAM system utilizes an engineered version of the CRISPR enzyme, in which the enzymatic activity required to cut DNA has been removed and replaced with a transcription factor recruitment activity to temporarily increase transcription of a desired gene. While the SAM system has the potential to allow rapid and prolonged alteration in gene expression, it cannot be directly injected, as biological systems rapidly degrade enzymes and nucleic acids. Successful delivery of the SAM system requires a nanocarrier, and the modular design of the protocell platform is ideal. The mesoporous silica core has variable size and shape to direct biodistribution and a controlled pore size and surface chemistry to accommodate diverse cargos. The supported lipid bilayer can be modified with targeting and trafficking ligands to effect selective binding and endosomal escape of cargo, while maintaining in vivo colloidal stability. The protocell for nucleic acid delivery has an efficiency of plasmid delivery and expression to rival the commercial agent, lipofectamine, but the protocell demonstrates much lower toxicity. As discussed herein, the loading and delivery of plasmids encoding a SAM system increased expression of p53, a highly regulated gene.

Identification of optimal gene targets for adaptation to extreme altitude, cold and heat, as well as increased athletic endurance. The SAM system has never been developed for adaption to environmental exposure or endurance before.

SAM delivery by protocells utilizing both targeted and non-targeted formulations of the platform. The SAM systems that provide the strongest gene expression will be packaged as plasmids into targeted or non-targeted protocells and tested in relevant cell lines for transfection efficiency and specificity, gene expression, and toxicity, as well as circulation behavior, stability and transfection efficiency ex ovo.

Determination of duration of gene expression increase. By utilizing the SAM plasmid system and protocells, alteration in gene expression should be both rapid and durable.

Effect of SAM system delivered by protocells on cell stress response. Targeted cells transfected with SAM systems are exposed to cell stressors and the effect on metabolism and survival monitored. As each of the adaptive modifications can act both in the directly altered cell and downstream through secreted proteins we will examine both responses.

### Protocell Delivery of CRISPR SAM Systems for Rapid On-Demand Adaptation to Extreme Environments and Enhanced Performance

Human adaptation to new environments occurs slowly through biological evolution or more rapidly through acclimatization, wherein environmental exposure leads progressively to adaptive alterations in gene expression; these alterations may allow an individual to function under extreme conditions to which the individual has never been exposed. For example, extreme high altitude exposure can result in acute mountain sickness (AMS), which affects many individuals at altitudes over 8,000 ft and results in headache, nausea, vomiting, dizziness, decreased energy and insomnia and an overall decrease in performance. Although not all individuals are able to adjust to extreme environments, slow increase in altitude over a period of several weeks to months can lead to reversible alterations in gene expression that can allow an individual to handle extreme altitude. Alterations in gene expression have also been found that allow individuals to adapt to high heat and extreme cold. For example, gene expression alterations reduce shivering thermogenesis and up-regulate non-shivering thermogenesis, an adaptation necessary for efficient energy usage in extreme cold. Other less extreme examples are increases in athletic endurance due to high altitude training, which can result in more efficient oxygen usage in the body and metabolism in muscle. However, these alterations usually require weeks to months to occur which limits the ability of a human to rapidly move between environments or to rapidly alter muscle endurance.

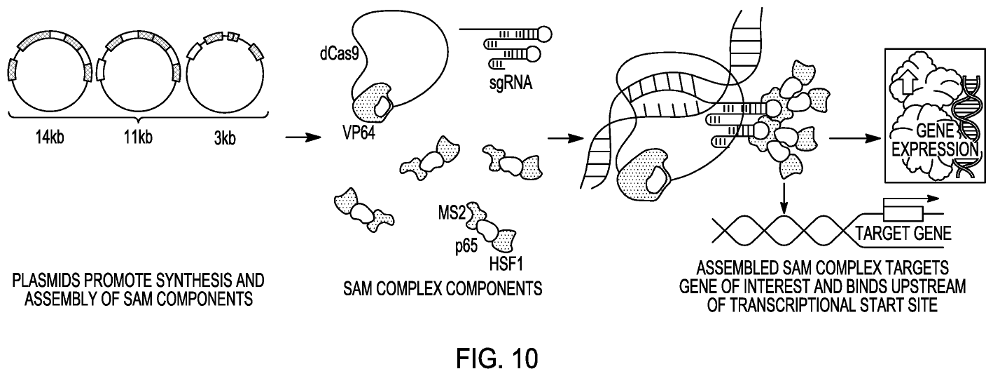

Rapid ‘on-demand’ alteration in the expression of these genes can be induced by utilizing CRIPSR-enabled technologies and could allow humans to pre-adapt to extreme conditions or increase endurance for weeks or months without the need for slow, progressive increase in environmental exposure or high altitude training. The CRISPR enzyme, in combination with targeted guide RNAs, is most commonly used to delete or edit genes creating permanent alterations in cellular genomes. However, the targeting is not perfect and can result in deleterious mutations as well as the desired editing. Additionally, for adaptations, such as those needed for performance enhancement under extreme environmental exposures, a reversible effect would be desirable as the same individual may want different adaptations in different scenarios. An engineered version of the CRISPR enzyme, in which the enzymatic activity required to cut DNA has been removed and replaced with a transcription factor recruitment activity, can be used in combination with targeted guide RNAs to temporarily increase transcription of a desired gene, this system is called the CRISPR Synergistic Activation Mediator (SAM) system (FIG. 10). By delivering the guide RNAs and the enzyme directly referred to as the ribonucleoprotein (RNP) complex, the increased gene expression would be extremely temporary, lasting only a matter of a few days to a week. However, prolonged gene expression can be maintained by delivering plasmids encoding for the CRISPR SAM system. Delivery of the plasmids should increase gene expression for weeks or months, allowing prolonged gene expression increase necessary for longer exposures to extreme environments. While the CRISPR SAM system has great potential to allow rapid and prolonged alteration in gene expression, the CRISPR SAM system, in either the enzyme or plasmid formats, cannot be directly injected into an organism and produce the desired effect. Biological systems rapidly degrade and clear both enzymes and nucleic acids, including RNA and DNA. To successfully deliver the CRISPR SAM system requires a nanocarrier. While there are many possible nanocarriers, the protocell platform is ideal for the delivery of the CRISPR SAM system.

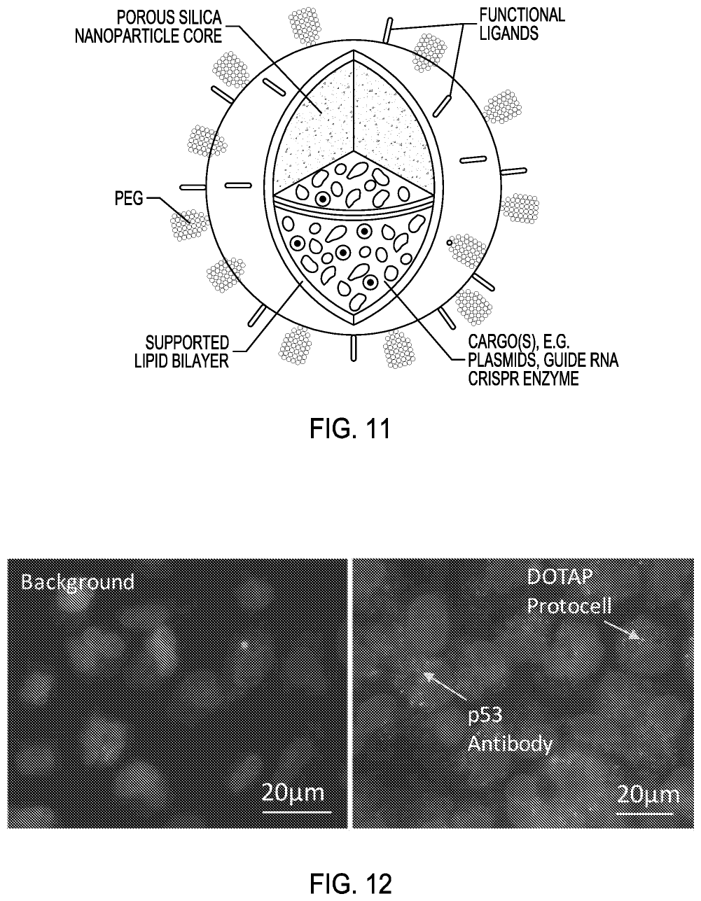

Mesoporous silica nanoparticle-supported lipid bilayers (SLBs), termed ‘protocells,’ represent a potentially transformative class of nanocarrier delivery vehicle (FIG. 11). The field of targeted delivery poses considerable challenges that cannot be addressed with a single ‘magic bullet’. Consequently, the protocell has been designed as a modular platform composed of interchangeable biocompatible components. The mesoporous silica core has variable size and shape to direct biodistribution and a controlled pore size and surface chemistry to accommodate diverse cargo. The encapsulating supported lipid bilayer can be modified with targeting and trafficking ligands as well as polyethylene glycol (PEG) to effect selective binding, endosomal escape of cargo, drug efflux prevention, and potent therapeutic delivery, while maintaining in vivo colloidal stability. In addition to the cargos discussed in the protocell platform reviews, utilizing the protocell platform we have recently successfully delivered plasmids utilizing both targeted and non-specific supported lipid bilayers to a variety of cell lines. We have optimized the protocell for nucleic acid delivery and find the efficiency of plasmid delivery and expression to rival that found with the commercial transfection agent, lipofectamine, but the protocell demonstrates much lower cellular toxicity and can therefore be developed for in vivo delivery. Loading and delivery of plasmids encoding a SAM system has been demonstrated (FIG. 10) to increase expression of p53. P53 is a highly regulated gene, whose expression is tightly controlled. By utilizing the protocell to package the p53 CRISPR SAM, we were able to increase expression of p53 within several cell lines (FIG. 12).

Capitalizing on the recent advances in our laboratory that allow for highly efficient delivery of plasmids and our success with the p53 CRISPR SAM construct, we propose to utilize the protocell to deliver SAM systems targeted to genes whose expression has been shown to be involved in adaptation to extreme environments and in increased endurance in athletes.

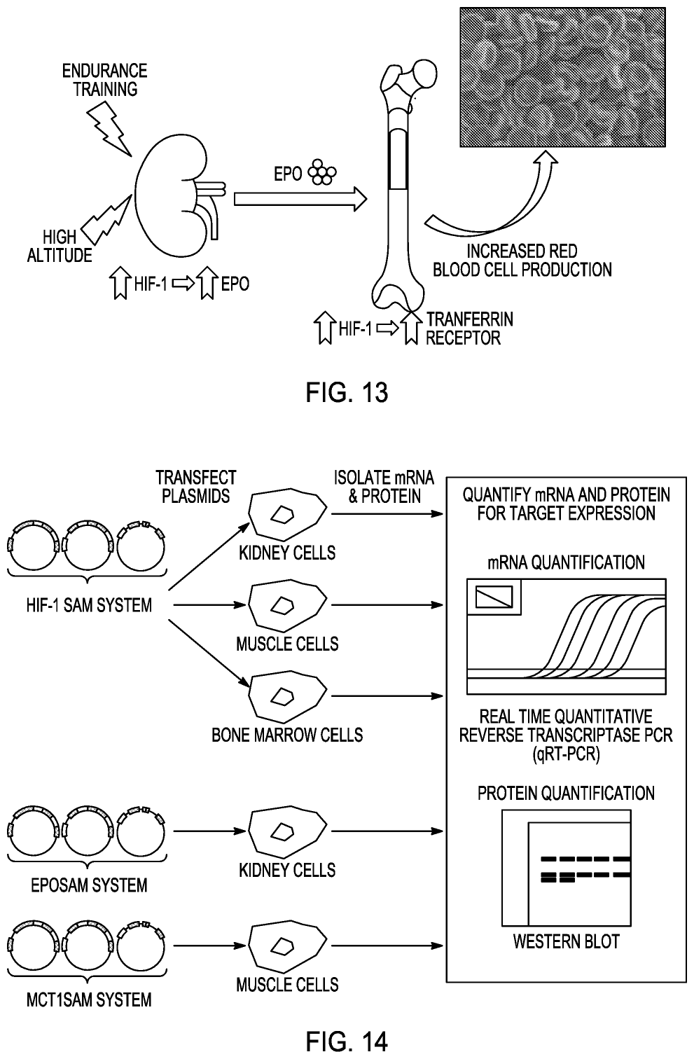

Identification of optimal target genes. In each of the proposed adaptations; extreme altitude, cold and heat as well as increased athletic endurance, multiple genes have been found to have increased expression in human adaptation. Some of these genes affect multiple downstream targets. For example, in cases of increased endurance, hypoxia inducible factor 1 (HIF-1) has increased expression. HIF-1 affects the expression of many downstream targets, some of which may not be required for the desired effect. However, one of the targets, the gene that encodes for erythropoietin (EPO) may be sufficient to show increased endurance and in fact is considered to be an illegal doping agent for athletes (FIG. 13). Therefore, increased expression of multiple genes in these pathways, both those that are early within the adaptation cascade and alter multiple cellular pathways as well as their downstream targets. Is tested. For each of the proposed adaptations, a selection of tissue specific cell lines will be utilized. While CRISPR SAM systems have never been developed for adaption to environmental exposure before, CRISPR SAM systems likely can be developed for enhanced endurance, resistance to cold and hypoxia.

Simulation of endurance training cellular response through CRISPR SAM system. Training at altitude or long duration endurance training results a mild hypoxia response that causes increased HIF-1 protein expression in many tissues. This increased expression has tissue specific responses, for example, HIF-1 increase in the kidney results in increased EPO production, which is then secreted from the kidney and stimulates erythrocyte production in bone marrow (FIG. 13). HIF-1 in bone marrow results in increased expression of transferrin receptors in erythrocyte precursors necessary for increased hemoglobin production necessary to compliment the enhanced erythrocyte production. Together, this system results in increased red blood cell mass which provides more efficient oxygen transport in humans. In addition to altering red blood cell production, the expression of HIF-1 in muscle cells results in increased production of monocarboxylate transporter 1 (MCT1) in skeletal muscle. MCT1 is involved in the transport of lactate and H+ions produced during anaerobic exercise metabolism and increased expression of MCT1 leads to optimized anaerobic muscle metabolism and reduced muscle fatigue through alterations in lactic acid transportation associated with enhanced running performance due to training at altitude.

The modular SAM system consists of 3 plasmids; on coding for the deactivated Cas9/VP64 fusion protein, one coding for the MS2/p65/HSF1 fusion protein and the final plasmid codes for the guide RNA (FIG. 10). The first two plasmids are essential for any SAM activation, while the guide RNA provides the gene specificity for selective activation. As optimal activation can vary based on the guide RNA sequence and the distance from the promoter, 3 sequences will be selected for each target gene and tested to identify the optimal activation system. To address the tissue specific nature of these adaptations, kidney cells, bone marrow cells and muscle cells will be tested for the increased expression of HIF-1. As EPO expression and MCT-1 expression are also anticipated to be tissue specific the expression of these genes will also be assessed in relevant cells specifically, the EPO production will be assessed in kidney cells both after the exposure to HIF-1 targeted CRISPR SAM and EPO targeted CRISPR SAM and the increase in MCT-1 will be assessed in muscle after HIF-1 SAM exposure (FIG. 14). Alterations in expression will be quantified in both mRNA and protein levels using qRT-PCR and western blot respectively.

Simulation of hypoxia cellular adaptive response through CRISPR SAM. EPO and HIF-1 expression are also important for hypoxia response for extreme altitude and operative trauma, but other genes also display increased expression including apelin and angiopoietin-like 4 (ANGPTL4) and play important roles in hypoxia resistance and high altitude endurance. ANGPTL4 is a serum hormone that is created in fat, liver and kidney as well as muscle, although the primary source in humans is liver. Increased expression of ANGPTL4 has been associated with hypoxia exposure in soldiers and mice and increased expression of ANGPTL4 utilizing a drug has been shown to significantly reduce cerebral edema in response to hypoxia. ANGPTL4 acts directly to counter the loss of vascular integrity and increased vascular permeability due to hypoxia through direct interaction and signaling in endothelial cells, and secretion of ANGPTL4 counteracts the vascular leakiness that leads to pulmonary and cerebral edema and results in headache, dizziness and reduced mental performance under high altitude hypoxia. Similar to ANGPTL4, apelin is a serum peptide that is created in many tissues including liver, kidney and muscle. Apelin and the apelin receptor are part of the adaptive response to high altitude and are critical for vascular and oxygen homeostasis and restoration of cellular O2 levels in hypoxic conditions. Apelin is upregulated in response to hypoxic conditions and the expression of apelin has been shown to have a protective effect on cells exposed to hypoxic conditions and animal models exposed to chronic hypoxia.

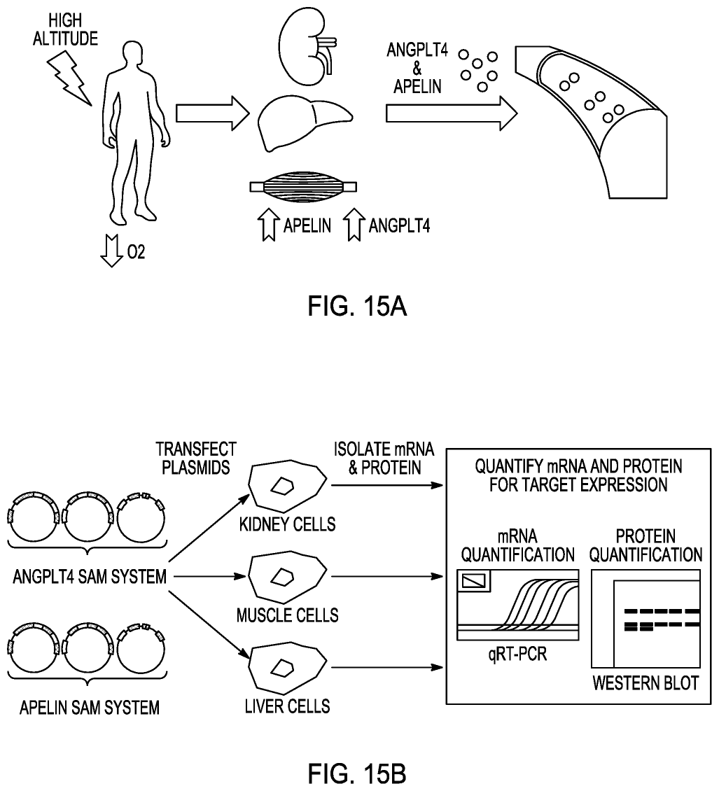

Following a similar experimental scheme, CRISPR SAM systems will be designed to target ANGPTL4 and apelin (FIG. 15). These SAM systems will be transfected into cells and the expression increase of both apelin and ANGPTL4 at both the mRNA and protein level will be assessed within cell lines derived liver, muscle and kidney. Protein levels will be monitored both within the cell and in the media, as both ANGPTL4 and apelin are secreted to fulfill their adaptive roles.

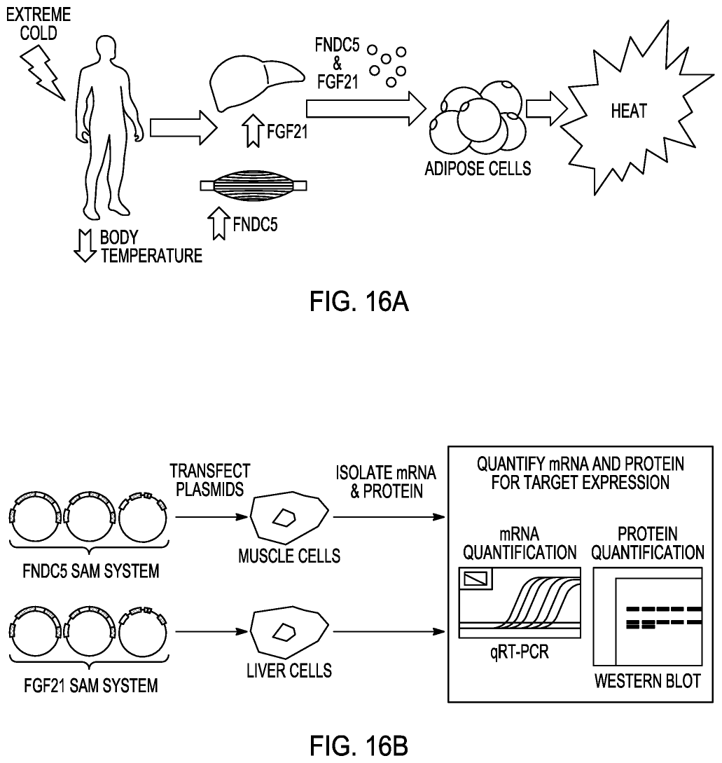

Cold acclimatization through CRISPR SAM system. Cold adaptation is the least well understood of the proposed adaptations, although it is well known that man can adapt to cold environments. Exposure of humans to consistent cold temperatures resulted in initial shivering that decreased significantly after 10 days of exposure. Muscle fasciculation, i.e., shivering, results in heat generation, called shivering thermogenesis (ST), which helps maintain core body temperature, however this involuntary defense mechanism is not sustainable in the long term. Over long-term exposure to cold, thermogenesis in humans switches from ST to non-shivering thermogenesis (NST), which results in maintenance of core body temperature with a more modest increase in energy expenditure than ST. Increased heat production, necessary for thermal adaptation, can be stimulated by the treatment of adipose cells with fibroblast growth factor 21 (FGF21) or fibronectin-type III domain-containing 5 (FNDC5). FNDC5 is a precursor protein for irisin, a hormone released from skeletal muscle and shown to be significantly increased after cold exposure and shivering (FIG. 16A). Similarly, FGF21 is an endocrine hormone that is secreted from liver in response to cold exposure (FIG. 16A). Similar to the experimental plan detailed in FIGS. 15 and 16, SAM systems will be designed for FNDC5 and FGF21 and these systems will be transfected into the target cells (FIG. 16B). In the case of FNDC5, the target cells are muscle cells and in the case of FGF21 the target cells are liver cells. The increased expression of FNDC5 and FGF21 will be examined at the mRNA level by quantitative RT-PCR and at the protein level by western blot. Protein levels are monitored both within the cell and in the media, as both FNDC5 and FGF21 are secreted to fulfill their roles cold adaptation.

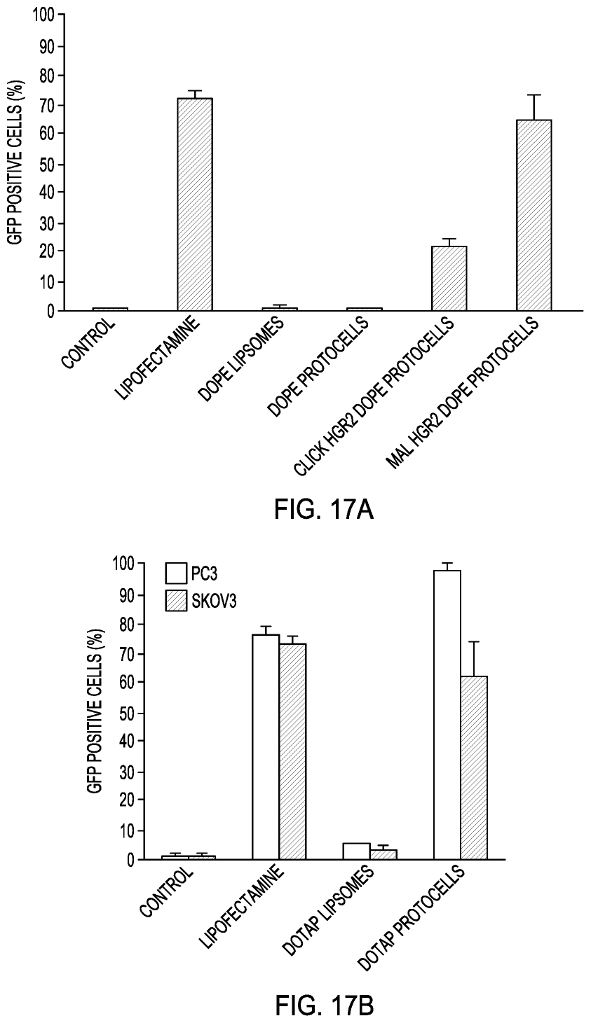

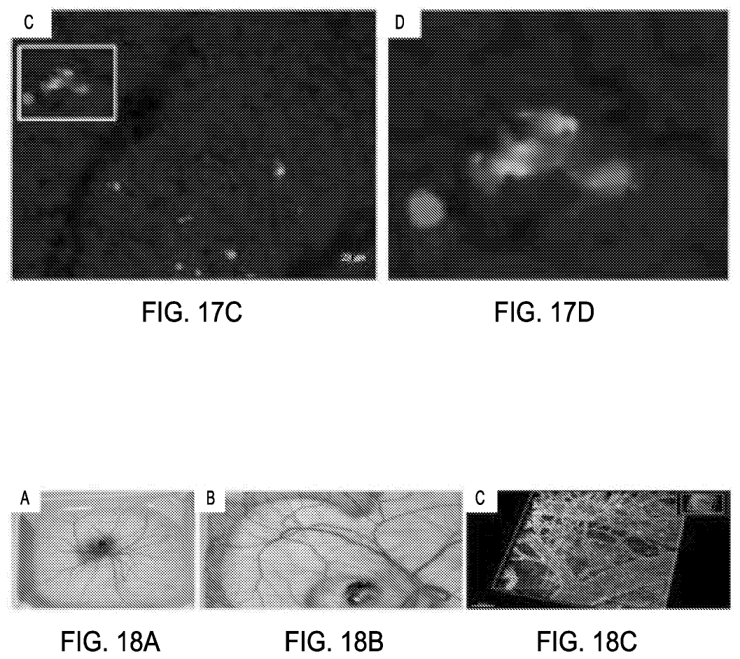

Delivery by protocells utilizing both targeted and non-targeted formulations of the platform. Once the CRISPR SAM systems that provide the strongest expression are identified, they will be packaged into the protocell system (FIG. 11). As the target tissue and the cells may vary, both targeted and non-targeted protocell formulations are examined. While targeting the protocells will provide specific delivery to the cells of interest, this added complexity can decrease the transfection efficiency and may not be necessary in all cases. For example, nanoparticles injected I.V. naturally accumulate in the liver, so ‘passive’ targeting of the liver is possible, similarly intramuscular injections would provide a direct route for delivery to muscle cells. Additionally, the delivery the CRISPR SAM system targeting a factor that is commonly increased in many cell types, such as HIF-1, may be more effective with systemic and non-specific delivery without any negative effects. Preliminary work has identified both targeted and non-specific lipid formulations that provide reliable plasmid transfection of cells as demonstrated by expression of GFP employing the plasmid system (FIG. 17). Experimentation with targeted delivery, using affibodies to Her2 has demonstrated that the percent of transfected cells can vary by the conjugation method used to attach the targeting agent (FIG. 17A). However successful targeting has been shown to allow transfection of up to about 70% of exposed cells, which is roughly equivalent to high doses of the commercial transfection agent, lipofectamine, but without the significant toxicity that occurs with use of lipofectamine. Additionally, the non-specific, cationic lipid formulation based on DOTAP that was used to deliver both GFP plasmid (FIGS. 17B-D) and the CRISPR SAM system to p53 (FIG. 12) using stable protocells, was used. DOTAP based protocells demonstrated transfection efficiency equivalent to lipofectamine, but without the toxicity, and significantly higher transfection than that of the DOTAP based lipids alone in multiple cell lines (FIG. 17B). Injection of even modest amounts of DOTAP based protocells loaded with GFP plasmid demonstrated transfection in a vascular model, the chicken chorioallantoic membrane (CAM), by 24 hours post injection (FIGS. 17C-D). The CAM model is a vascularized tissue that allows direct, real-time intravital imaging of nanoparticles in blood flow (FIG. 18). This model can be utilized to observe nanoparticle circulation, flow characteristics, non-specific interactions, and particle stability in a living system; furthermore, the CAM system can be utilized to examine the characteristics of targeted protocell binding to targeted cells using real-time intravital imaging.

As the expression level can vary with the non-targeted and targeted protocells, both targeted and non-specific delivery by protocells to multiple cell linesis tested to determine the robustness of the increased expression. Testing occurs within cell lines matched for the intended expression target, and targeting agents are utilized based on the selected genes and cell types. As the protocells are anticipated to both circulate well within the vascular system and associate with the cells of interest either non-specifically or in a targeted manner the protocells and their targeting are evaluated in the CAM.

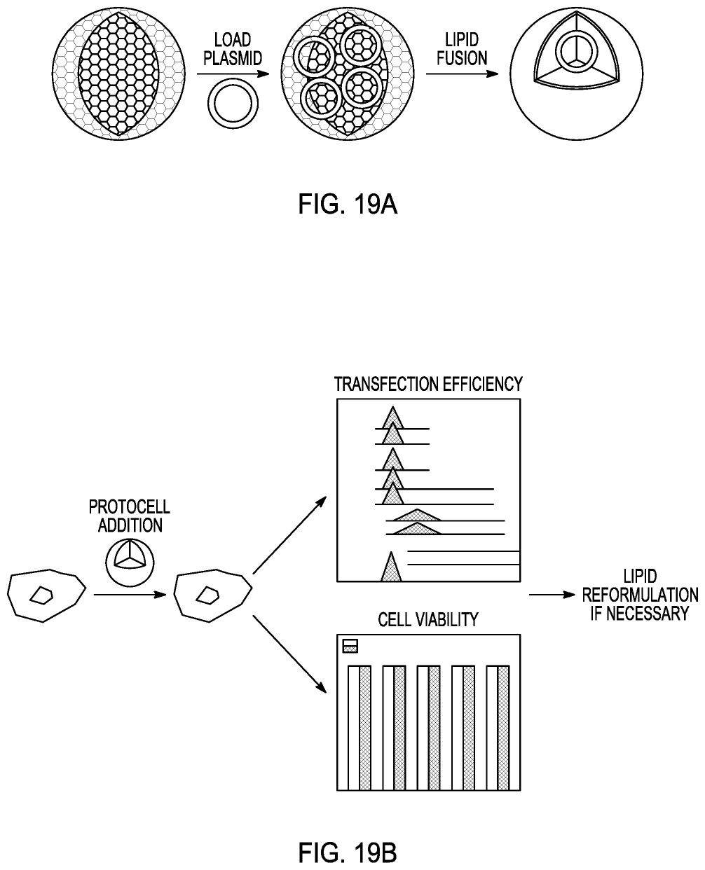

Testing of non-specific, cationic lipid based protocell transfection. As the majority of transfection studies, including studies utilizing the protocell, to date have focused on epithelial cells, which would include kidney and liver, but not bone marrow and muscle cells, transfection studies are inititated using a GFP plasmid. Utilizing the same cell lines, DOTAP based protocell formulations are tested for both transfection efficiency and toxicity. By initiating these studies, lipid formulation are assessed for the formulation that provides the highest transfection efficiency and the lowest toxicity across multiple cell types, as well as to determine if different formulations are needed for cells of different origins (FIG. 19).

Testing of targeted protocells for transfection. Successful targeting with full length antibodies, scFvs, affibodies (FIG. 17A), small molecules, and peptides may be employed, among other targeting moieties for transfection of the CRISPR SAM system into the target cells of interest. Additionally, lipid formulations that facilitate delivery of plasmids while allowing specific targeting (FIG. 17A) may be employed. Bone marrow, liver, kidney and muscle targeted protocells are prepared and tested for specific binding and transfection efficiency utilizing a GFP plasmid. Targeting is aimed toward cell surface receptors that are expressed on the target cells and will be selected from the literature. In the case of bone marrow, the intended target cells are hematopoietic cells and protocell specificity will be achieved by targeting either erythropoietin receptor or the transferrin receptor on the cell surface of the hematopoietic cells. In the case of liver cells, lactose is an effective targeting moiety and readily conjugated to lipids. Skeletal muscle cell delivery will be achieved by conjugation of either the small molecule I-carnitine, antibodies to the organic cation/carnitine transporter (OCTN2) or muscle targeting peptides. Finally, in the case of kidney targeting, specific cell types must be targeted depending on the desired alteration and desired cell type. For example, EPO is produced in the renal peritubular cells and these cells can be targeted by use of anti-CD73 antibodies. ANGPLT4 is expressed in the podocytes in the kidney and protocells will be targeted to podocytes utilizing antibodies to nephrin or Vascular Cell Adhesion Molecule-1 (VCAM-1). Apelin is expressed in the vascular endothelial cells of the kidneys and these cells can be targeted utilizing antibodies to VCAM-1, intercellular adhesion molecule-1 (ICAM-1) or E-selectin. The developed targeted protocells will be assessed for specific binding, cytotoxicity and transfection efficiency using a GFP plasmid to identify the most successful protocell constructs (FIG. 11). Once the CRISPR SAM constructs have been tested and the top candidates identified, these constructs are loaded into the protocells with the non-specific lipid formulations.

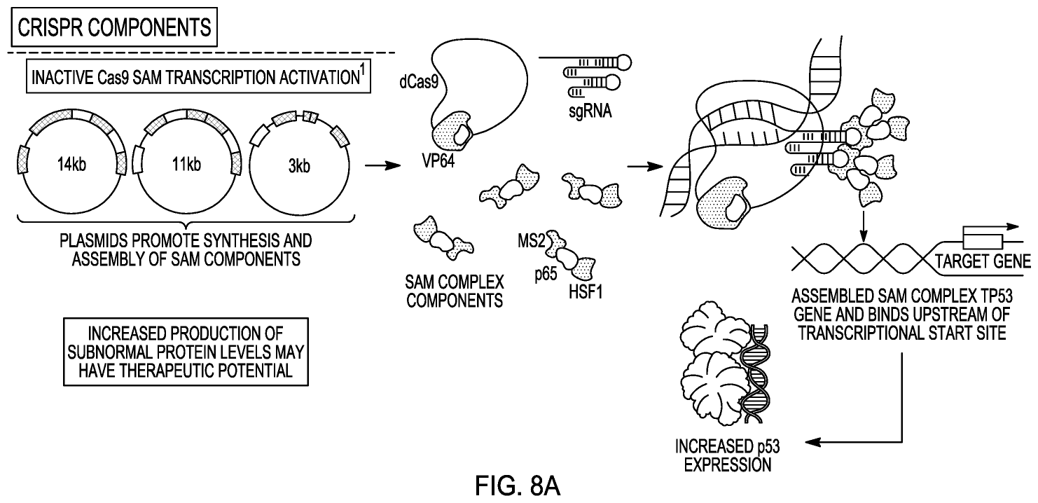

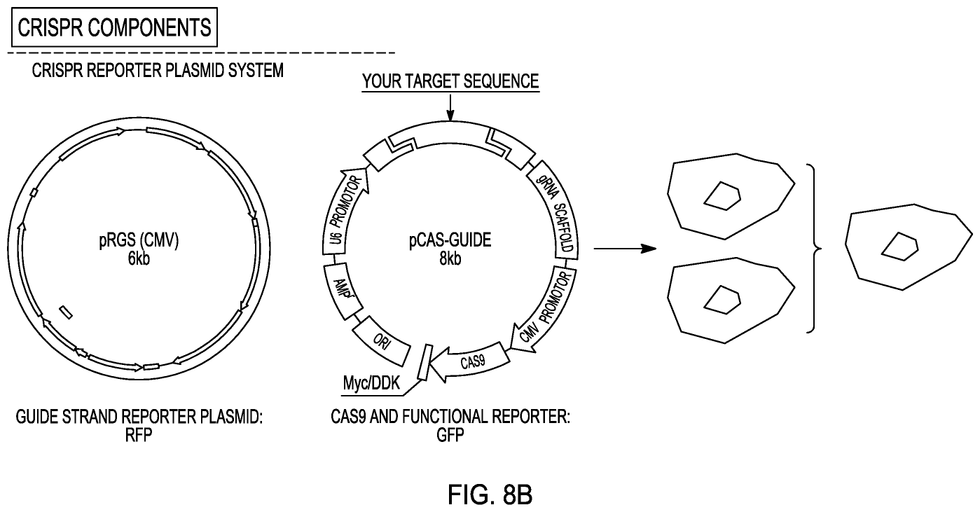

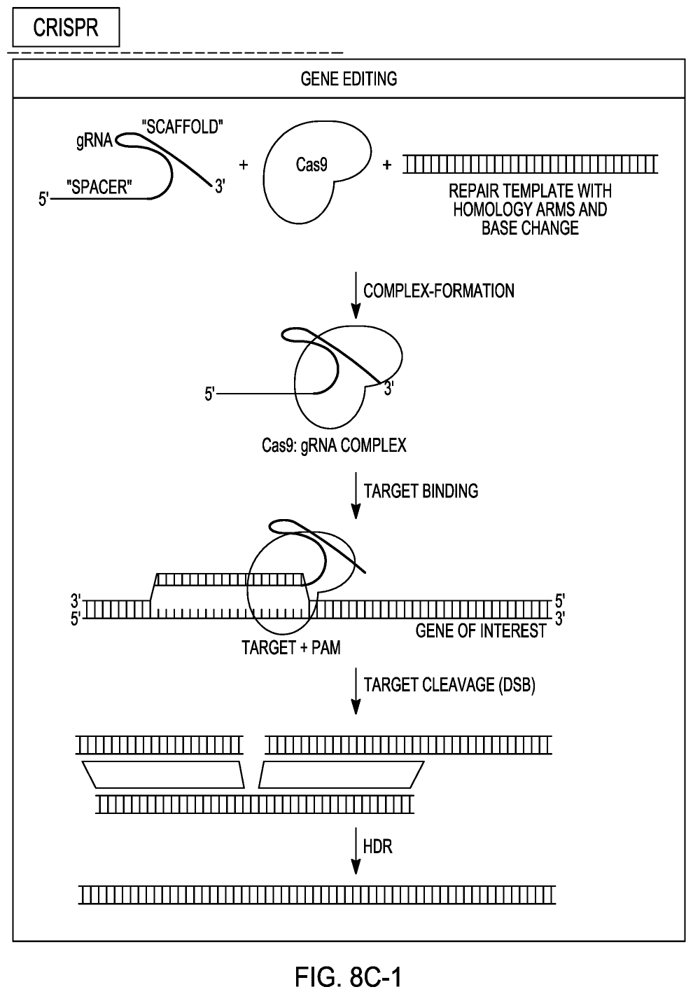

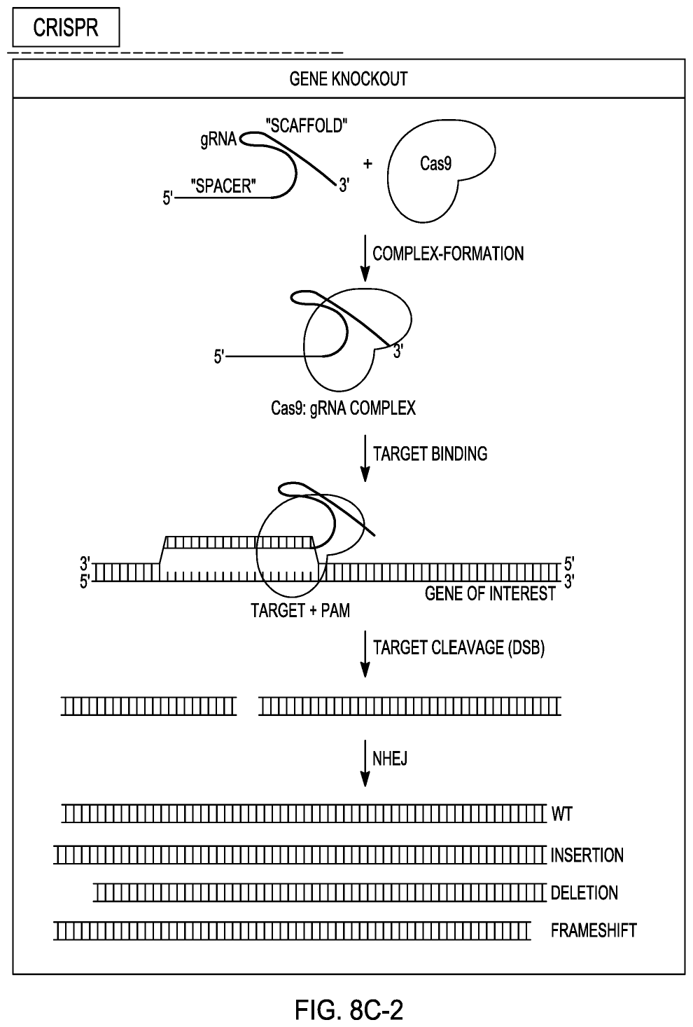

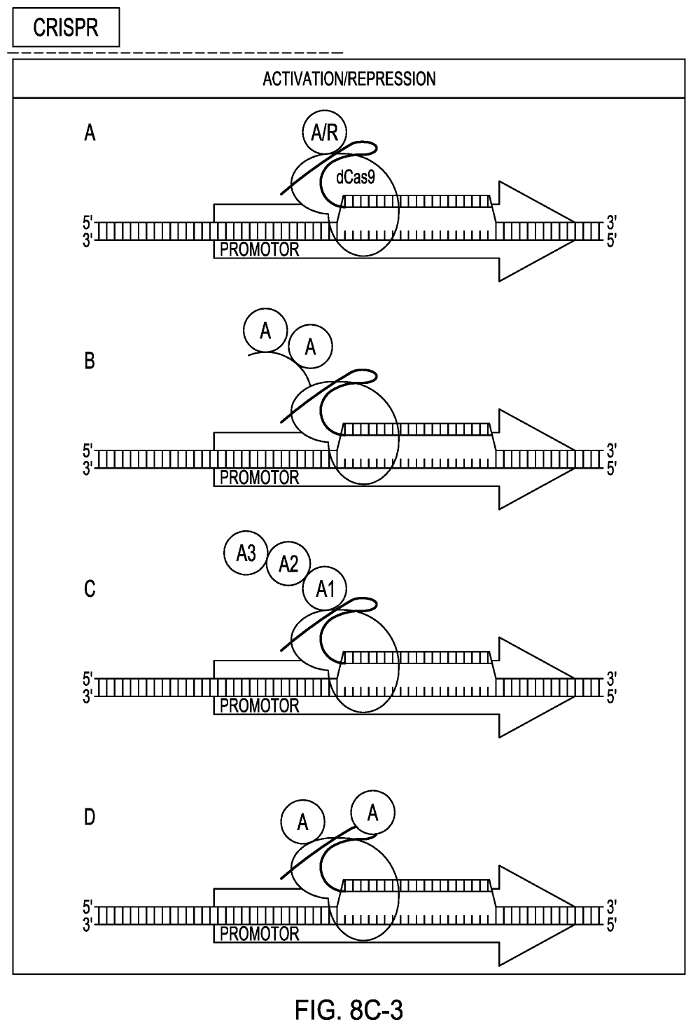

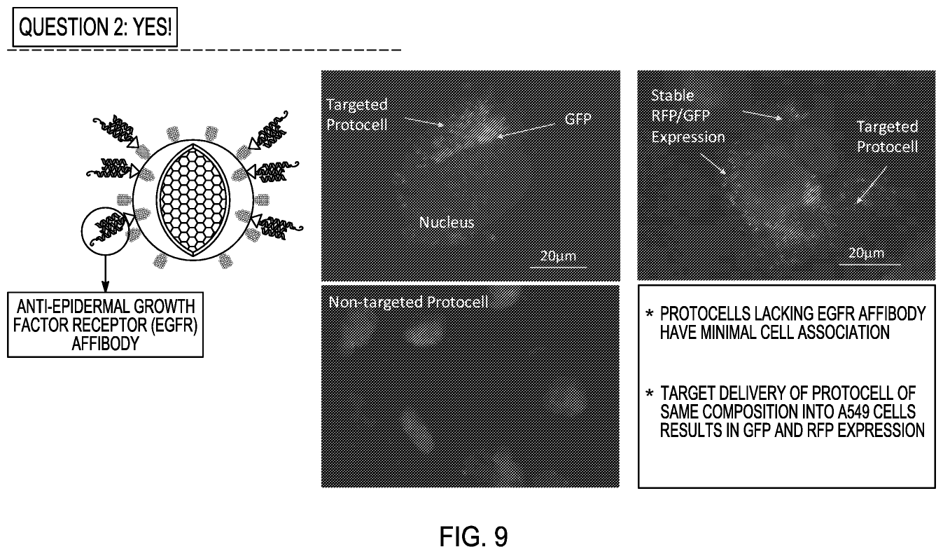

Evaluation of protocells for circulation and binding to target cells using the CAM system. As the protocells are designed for eventual in vivo use, their vascular flow, selective binding and delivery of cargo is evaluated within the CAM system. Stable monosized protocell circulation and flow as well as specific cell targeting and cargo delivery within the CAM system has been demonstrated (FIG. 12). Utilizing this model, the flow characteristics of each of designed protocells is evaluated via intra-vital imaging to ensure that the designed protocells circulate well within the vascular space by injecting the protocells into the vascular system and observing in real time the interactions with the endothelial cells of the vessel walls and the duration of circulation (FIG. 12A). Once vascular stability is established, the targeted protocells are tested for the ability to interact specifically with the cell of interest. Target cells are labeled with a vital dye that is retained within the cell for several days and then inject them into the CAM. A second injection is performed of the protocells. The specific binding and the time required for binding of the protocells can be tested using, e.g., membrane impermeant dyes (FIG. 12B) or plasmids (FIG. 8 C&D) in real-time using the CAM system and intravital imaging. The GFP plasmid can assess both targeted and non-targeted plasmid delivery in the CAM system.

Duration of gene expression increase. The advantage of the use of the CRISPR SAM system combined with protocell delivery is the rapid ‘on-demand’ alteration in the expression of genes. By utilizing the CRISPR SAM plasmid system, this alteration should be rapid and also durable over a short time frame allowing a single dose to provide alterations in gene expression for weeks to months and then allowing expression to return to baseline. Previous experimentation with the protocell platform as a delivery vehicle for plasmid delivery to neuronal tissue has shown transfection up to 8 weeks post injection. However, the expression level and duration can vary based on tissue type and even transfection method. One of the major reasons for this variability is the nature of the plasmid itself. Plasmids are not normally present within mammalian systems, being native to bacteria, and are not normally replicated when the chromosomes are replicated for cell division. Therefore the plasmid load within a group of cells is slowly lost overtime and the rate of loss is relative to the rate of cellular proliferation. The rate of cellular proliferation is tissue/cell type dependant and accounts for the very rapid loss of plasmid expression in liver cells compared to muscle tissue as the rate of proliferation of liver cells exceeds muscle cells. However, the transfection method is also a factor, as a more efficient transfection results in more copies of plasmid per cell and therefore more replications before the plasmid expression is lost. As targeting multiple cell types, including; muscle, liver, kidney and bone marrow, is envisioned, the duration of the expression may vary greatly between these cell types and may also vary between targeted and non-targeted protocells. Therefore, the duration of the gene expression increase is monitored to estimate the durability of this response in each of the selected genes and in multiple cell lines based on the cell lines and utilizing the protocell constructs designed and tested. Monitoring for increased expression of the gene of interest is done both at the mRNA level by real-time quantitative PCR and at the protein level by western blot.

Effect of SAM system delivered by protocells on cell stress response. Finally, the targeted cells transfected with CRISPR SAM systems by protocells are exposed to various cell stressors and monitor the effect on metabolism and survival. Each of the adaptive modifications targeted by the SAM CRISPR systems and delivered by protocells are designed to provide rapid adaptation to an environmental stressor and can act both in the directly altered cell and downstream through secreted proteins. Since both a direct effect on the transfected target cells and a downstream effect on other cells through secreted proteins are expected, both responses are examined (FIG. 13). While the exact cells and CRISPR SAM systems chosen will vary, an example experimental set up for cold adaptation would be as follows. Muscles cells are transfected by protocells loaded with the SAM system targeting FNDC5. The cells then produce FNDC5 and excrete this into the media. Media is collected from the muscle cells and added to adipose cells, the downstream target cell of FNDC5. Both the muscle cells and the adipose cells are then exposed to reduced temperature incubation and assessed for metabolic changes and cell viability to determine if the FNDC5 CRISPR SAM system increased survivability or metabolic activity compared to untreated cells. Cell stressors to be examined include reduced incubation temperature, reduced oxygen (hypoxia) and decreased pH to mimic endurance exercise.

Assessment of the cross protection from one adaptation to other cellular stresses. Cells can use increases in expression of a protein associated with one cellular stressor, for example heat shock proteins for adaptation to extreme heat, to cross-protect against other stressors, most commonly toxins like arsenite or bacterial lipopolysaccharide. While the level of cross-protection has not been fully explored, it provides the possibility that some of the proposed adaptations may provide an individual protection against toxic exposures as well as the on demand' expected environmental stressor. Examination of this cross-protection effect could potentially identify a single gene that could be upregulated utilizing an on-demand system and provide adaptation to multiple extreme environments as well as increased endurance or accidental toxic exposure. After the completion of in vitro testing in cell lines, the next step is animal studies. In each of these adaptations there are known rodent models that can be utilized for functional assessment of the protective effects of the CRISPR SAM constructs delivered by protocells.

The CRISPR SAM system is a new and novel system recently developed to allow targeted increase in gene expression. The methods and data described herein lay the groundwork for the expansion of the CRISPR SAM system into a wide range of applications and develop fundamental knowledge in the use of the CRISPR SAM system. Additionally, the project results in the development of a modular delivery system for nucleic acids based on protocells and which could essentially be adapted for both tissue specific and cell specific delivery and provide the necessary stability for plasmid delivery in vivo.

### REFERENCES

1 Carroll, N. J., Pylypenko, S., Atanassov, P. B. & Petsev, D. N. Microparticles with Bimodal Nanoporosity Derived by Microemulsion Templating. Langmuir, doi:10.1021/Ia900988j (2009).
2 Lu, Y. F. et al. Aerosol-assisted self-assembly of mesostructured spherical nanoparticles. Nature 398, 223-226 (1999).
3 Iler, R. K. The Chemistry of Silica: Solubility, Polymerization, Colloid and Surface Properties, and Biochemistry, (John Wiley and Sons, 1979).
4 Doshi, D. A. et al. Neutron Reflectivity Study of Lipid Membranes Assembled on Ordered Nanocomposite and Nanoporous Silica Thin Films. Langmuir 21, 2865-2870, doi:10.1021/Ia0471240 (2005).
5 Bernhard, M. I. et al. Guinea Pig Line 10 Hepatocarcinoma Model: Characterization of Monoclonal Antibody and in Vivo Effect of Unconjugated Antibody and Antibody Conjugated to Diphtheria Toxin A Chain. Cancer Research 43, 4420-4428 (1983).
6 Lo, A., Lin, C. T. & Wu, H. C. Hepatocellular carcinoma cell-specific peptide ligand for targeted drug delivery. Molecular Cancer Therapeutics 7, 579-589, doi:10.1158/1535-7163.mct-07-2359 (2008).
7 Sciot, R. et al. Transferrin receptor expression in human hepatocellular carcinoma: an immunohistochemical study of 34 cases. Histopathology 12, 53-63 (1988).
8 Kannangai, R., Sahin, F. & Torbenson, M. S. EGFR is phosphorylated at Ty845 in hepatocellular carcinoma. Mod Pathol 19, 1456-1461 (2006).
9 Behr, J. P. The Proton Sponge: a Trick to Enter Cells the Viruses Did Not Exploit. CH/MIA International Journal for Chemistry 51, 34-36 (1997).
10 Jiang, W., KimBetty, Y. S., Rutka, J. T. & ChanWarren, C. W. Nanoparticle-mediated cellular response is size-dependent. Nat Nano 3, 145-150 (2008).
11 Zimmermann, R. et al. Charging and structure of zwitterionic supported bilayer lipid membranes studied by streaming current measurements, fluorescence microscopy, and attenuated total reflection Fourier transform infrared spectroscopy. Biointerphases 4, 1-6 (2009).
12 Ashihara, E., Kawata, E. & Maekawa, T. Future Prospect of RNA Interference for Cancer Therapies. Current Drug Targets 11, 345-360 (2010).
13 Pawitan, J. A. The possible use of RNA interference in diagnosis and treatment of various diseases. International Journal of Clinical Practice 63, 1378-1385 (2009).
14 Elbashir, S. M. et al. Duplexes of 21-nucleotide RNAs mediate RNA interference in cultured mammalian cells. Nature 411, 494-498 (2001).
15 Davis, M. E. et al. Evidence of RNAi in humans from systemically administered siRNA via targeted nanoparticles. Nature advance online publication (2010).
16 Oh, Y.-K. & Park, T. G. siRNA delivery systems for cancer treatment. Advanced Drug Delivery Reviews 61, 850-862 (2009).
17 Sou, K., Endo, T., Takeoka, S. & Tsuchida, E. Poly(ethylene glycol)-Modification of the Phospholipid Vesicles by Using the Spontaneous Incorporation of Poly(ethylene glycol)-Lipid into the Vesicles. Bioconjugate Chemistry 11, 372-379, doi:10.1021/bc990135y (2000).
18 Klein, E. etal. “HFP” Fluorinated Cationic Lipids for Enhanced Lipoplex Stability and Gene Delivery. Bioconjugate Chemistry 21, 360-371, doi:10.1021ibc900469z (2010).
19 Minguez, B., Tovar, V., Chiang, D., Villanueva, A. & Llovet, J. M. Pathogenesis of hepatocellular carcinoma and molecular therapies. Current Opinion in Gastroenterology 25, 186-194 110.10971MOG.1090b1013e32832962a32832961 (2009).
20 Li, S.-D., Chen, Y.-C., Hackett, M. J. & Huang, L. Tumor-targeted Delivery of siRNA by Self-assembled Nanoparticles. Mol Ther 16, 163-169, doi:http://www.nature.com/mt/journal/v16/n1/suppinfo/300323s1.html (2007).
21 Landen, C. N. et al. Therapeutic EphA2 Gene Targeting In vivo Using Neutral Liposomal Small Interfering RNA Delivery. Cancer Research 65, 6910-6918 (2005).
22 Honjo, T., Nishizuka, Y., Hayaishi, O. & Kato, I. Diphtheria Toxin-dependent

Adenosine Diphosphate Ribosylation of Aminoacyl Transferase II and Inhibition of Protein Synthesis. Journal of Biological Chemistry 243, 3553-3555 (1968).
23 Uchida, T., Kim, J. H., Yamaizumi, M., Miyake, Y. & Okada, Y. Reconstitution of lipid vesicles associated with HVJ (Sendai virus) spikes. Purification and some properties of vesicles containing non-toxic fragment A of diphtheria toxin. Journal of Cell Biology 80, 10-20 (1979).
24 Mizuguchi, H. et al. Application of fusogenic liposomes containing fragment A of diphtheria toxin to cancer therapy. British Journal of Cancer 73, 472-476 (1997).
25 Liu, J. W., Jiang, X. M., Ashley, C. & Brinker, C. J. Electrostatically Mediated Liposome Fusion and Lipid Exchange with a Nanoparticle-Supported Bilayer for Control of Surface Charge, Drug Containment, and Delivery. Journal of the American Chemical Society 131, 7567-+, doi:10.1021/ja902039y (2009).
26 Liu, J. W., Stace-Naughton, A. & Brinker, C. J. Silica nanoparticle supported lipid bilayers for gene delivery. Chemical Communications, 5100-5102, doi:10.1039/b911472f (2009).
27 Liu, J. W., Stace-Naughton, A., Jiang, X. M. & Brinker, C. J. Porous Nanoparticle Supported Lipid Bilayers (Protocells) as Delivery Vehicles. Journal of the American Chemical Society 131, 1354-+, doi:10.1021/ja808018y (2009).
28 Schagger, H. Tricine-SDS-PAGE. Nat. Protocols 1, 16-22 (2006).
29 Fritze, A., Hens, F., Kimpfler, A., Schubert, R. & Peschka-Süss, R. Remote loading of doxorubicin into liposomes driven by a transmembrane phosphate gradient. Biochimica et Biophysica Acta (BBA)—Biomembranes 1758, 1633-1640 (2006).
30 Elorza, B., Elorza, M. A., Frutos, G. & Chantres, J. R. Characterization of 5-fluorouracil loaded liposomes prepared by reverse-phase evaporation or freezing-thawing extrusion methods: study of drug release. Biochimica et Biophysica Acta 1153, 135-142 (1993).
31 Peleg-Shulman, T., Gibson, D., Cohen, R., Abra, R. & Barenholz. Y. Characterization of sterically stabilized cisplatin liposomes by nuclear magnetic resonance. Biochimica et Biophysica Acta 1510, 278-291 (2001).
32 Bogush, T., Smirnova, G., Shubina, L., Syrkin, A. & Robert, J. Direct evaluation of intracellular accumulation of free and polymer-bound anthracyclines. Cancer Chemotherapy and Pharmacology 35, 501-505, doi:10.1007/BF00686835 (1995).
33 Tong, A. W. et al. Chemosensitization of human hepatocellular carcinoma cells with cyclosporin A in post-liver transplant patient plasma. Clin, Cancer Res. 2, 531-539 (1996).
34 Minko, T., Kopecková, P. & Kopecek, J. Chronic exposure to HPMA copolymer-bound adriamycin does not induce multidrug resistance in a human ovarian carcinoma cell line. Journal of Controlled Release 59, 133-148 (1999).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A porous protocell comprising:
a nanoporous silica or metal oxide core with a supported lipid bilayer and one or more non-integrating vectors comprising nucleic acid encoding a non-nucleolytic fusion protein comprising a protein sequence that specifically binds a sequence in a double strand nucleic acid molecule linked to a protein sequence that is a transcriptional mediator, and optionally guide RNA or a vector for expression of guide RNA.

2-3. (canceled)

4. The protocell according to claim 1 wherein the protein sequence that specifically binds a sequence in a double strand nucleic acid molecule is N-terminal to the protein sequence that is a transcriptional mediator.

5. The protocell according to claim 1 wherein the protein sequence that specifically binds a sequence in a double strand nucleic acid molecule is C-terminal to the protein sequence that is a transcriptional mediator.

6. The protocell according to claim 1 wherein the vector encoding the fusion protein encodes guide RNA.

7. The protocell according to claim 1 wherein the protein sequence that specifically binds a double strand nucleic acid molecule is a Cas protein.

8. The protocell according to claim 1 wherein the protein sequence that is a transcriptional mediator comprises VP64.

9. The protocell of claim 1 wherein the gRNA is sgRNA comprising a base-pairing sequence, a dCas9-binding hairpin and a terminator.

10-12. (canceled)

13. The protocell of claim 1 wherein the gRNA is for p53HIF-1, EPO, MCT1, alpelin, ANGPLKT4, FGF21 or FNDC5.

14. The protocell of claim 1 wherein the transcription mediator comprises VP16, TA2, VP64 (a tetrameric repeat of the minimal activation domain of VP16), signal transducer and activator of transcription 6 (STATE), reticuloendotheliosis virus A oncogene (relA), TATA binding protein associated factor-1 (TAF-1), TATA binding protein associated factor-2 (TAF-2), glucocorticoid receptor TAU-1, or glucocorticoid receptor TAU-2, or a portion thereof having transcription activating activity.

15. The protocell of claim 1 wherein the transcription mediator comprises ETS repressor factor, the ETS repressor factor repressor domain (ERD), Kruppel-associated box (KRAB), human MAD1 protein, mSin3 interaction domain of the human MAD1 protein (SID), histone deacetylase, DNA methylase, or is a derivative or multimer of KRAB, SID, or ERD selected from the group consisting of KRAB-ERD, SID-ERD, (KRAB)₂, (KRAB)₃, KRAB-A, (KRAB-A)₂, (SID)₂, (KRAB-A)-SID, or SID-KRAB-A, or a portion thereof having transcription repressing activity.

16. The protocell of claim 1 which comprises two or more different unlinked vectors.

17. The protocell of claim 1 which comprises two or more different plasmids.

18. The protocell of claim 1 wherein the protein sequence that specifically binds the sequence comprises a transcription factor, Ga14, hypoxia inducible factor (HIF), e.g., HIF1α, cyclic AMP response element binding (CREB) protein, LexA, rtTA, an endonuclease, a zinc finger binding domain, or a synthetic DNA binding domains

19. The protocell of claim 1 wherein the protein sequence that specifically binds DNA comprises a RNA-guided DNA endonuclease that lacks endonuclease activity.

20-40. (canceled)

41. A method of treating cancer, diagnosing cancer or monitoring cancer therapy comprising administering to a patient in need an effective amount of a composition comprising a population of protocells according to claim 1 which have been adapted to deliver an anticancer agent to a cancer cell in said patient or to monitor cancer therapy adapted to selectively bind to cancer cells and deliver the protocells to said cells, wherein said protocells comprise a plasmid DNA adapted to express a reporter molecule and optionally comprise an additional reporter molecule, whereupon the binding of the protocell to a cancer cell in said patient will release said reporter molecules into the cancer cells, if present, and the reporter molecules will elicit a signal which can be compared with a standard to determine whether or not the patient has cancer and if so, the extent of the cancer and/or size of a cancerous tumor, if present or compared with a standard at the commencement of therapy and at varying intervals during the course of therapy to determine whether or not patient is responding to the therapy and if so, the extent of the response to the therapy.

42-43. (canceled)

44. A method to enhance a response to hypoxia or to excessive cold in a mammal, comprising administering to the mammal an effective amount of a composition comprising a population of protocells according to claim 1, wherein the protocells comprise a guide RNA or a vector for expression of guide RNA specific for a gene that is upregulated in response to hypoxia or to cold stress.

45. (canceled)

46. The protocell according to claim 1 wherein the transcriptional mediator is a transcriptonal activator.

47. The protocell according to claim 1 wherein the transcriptional mediator is a transcriptonal repressor.

48. The protocell of claim 46 further comprising a nucleic acid encoding an activator helper protein.

49. The protocell of claim 48 wherein the helper protein comprises MS2-P65-HSF1 or
wherein
the nucleic acid encoding an activator helper protein is on a different vector than the vector
comprising
the nucleic acid encoding the fusion protein.


SEQUENCE LISTING (19 sequences)

SEQ ID NO: 1 - protein, 7 aa, Artificial Sequence
  A synthetic polypeptide
ASVHFPP

SEQ ID NO: 2 - protein, 7 aa, Artificial Sequence
  A synthetic polypeptide
TATFWFQ

SEQ ID NO: 3 - protein, 7 aa, Artificial Sequence
  A synthetic polypeptide
TSPVALL

SEQ ID NO: 4 - protein, 7 aa, Artificial Sequence
  A synthetic polypeptide
IPLKVHP

SEQ ID NO: 5 - protein, 7 aa, Artificial Sequence
  A synthetic polypeptide
WPRLTNM

SEQ ID NO: 6 - protein, 12 aa, Artificial Sequence
  A synthetic polypeptide
SFSIILTPILPL

SEQ ID NO: 7 - protein, 15 aa, Artificial Sequence
  A synthetic polypeptide
SFSIILTPILPLGGC

SEQ ID NO: 8 - protein, 18 aa, Artificial Sequence
  A synthetic polypeptide
SFSIILTPILPLEEEGGC

SEQ ID NO: 9 - protein, 42 aa, Artificial Sequence
  A synthetic polypeptide
GNQSSNFGPMKGGNFGGRSSGPYGGGGQYFAKPRNQGGYGGC

SEQ ID NO: 10 - protein, 7 aa, Artificial Sequence
  A synthetic polypeptide
RRMKWKK

SEQ ID NO: 11 - protein, 7 aa, Artificial Sequence
  A synthetic polypeptide
PKKKRKV

SEQ ID NO: 12 - protein, 16 aa, Artificial Sequence
  A synthetic polypeptide
KRPAATKKAGQAKKKK

SEQ ID NO: 13 - protein, 25 aa, Artificial Sequence
  A synthetic polypeptide
GLFHAIAHFIHGGWHGLIHGWYGGC

SEQ ID NO: 14 - protein, 8 aa, Artificial Sequence
  A synthetic polypeptide
RRRRRRRR

SEQ ID NO: 15 - protein, 26 aa, Artificial Sequence
  A synthetic polypeptide
GLFHAIAHFIHGGWHGLIHGWYGGGC

SEQ ID NO: 16 - protein, 41 aa, Artificial Sequence
  A synthetic polypeptide
NQSSNFGPMKGGNFGGRSSGPYGGGGQYFAKPRNQGGYGGC

SEQ ID NO: 17 - skipped

SEQ ID NO: 18 - protein, 12 aa, Artificial Sequence
  A synthetic polypeptide
YLFSVHWPPLKA

SEQ ID NO: 19 - protein, 7 aa, Artificial Sequence
  A synthetic polypeptide
HAIYPRH